(19) United States
(12) Patent Application Publication
(10) Pub. No.: US 2024/0423691 A1
(43) Pub. Date: Dec. 26, 2024

(54) SURGICAL INSTRUMENT FOR ELECTROCAUTERY

(71) Applicant: LIVSMED INC., Seongnam-si (KR)

(72) Inventors: Dong Hoon KANG, Seoul (KR); Tae Hyun LIM, Hanam-si (KR); Mi Ryoung CHOI, Seongnam-si (KR); Jae Yeong LEE, Seongnam-si (KR); Tae Jin PARK, Seongnam-si (KR); Dong San LEE, Seoul (KR)

(21) Appl. No.: 18/751,163

(22) Filed: Jun. 21, 2024

(30) Foreign Application Priority Data
Jun. 22, 2023 (KR) 10-2023-0080329
Jul. 21, 2023 (KR) 10-2023-0095656
Dec. 1, 2023 (KR) 10-2023-0172386

(51) Int. Cl. A61B 18/08; A61B 18/00

(52) U.S. Cl. CPC A61B 18/085; A61B 2018/00077; A61B 2018/00083; A61B 2018/00178; A61B 2018/00202; A61B 2018/00595

(57) ABSTRACT

Provided is a surgical instrument for electrocautery including an end tool including one or more jaws and configured to be rotatable in two or more directions, a connection part extending in a first direction (an X-axis) and having one end to which the end tool is coupled, a wire having one end connected to the end tool, a shrink tube into which the other end of the wire, opposite to the one end of the wire connected to the end tool, is inserted, and a shaft tube, into which the other end of the shrink tube, opposite to one end of the shrink tube into which the wire is inserted, is inserted, wherein the wire and the shaft tube are separated by the shrink tube, and the wire and the shaft tube are insulated from each other.

### CROSS-REFERENCE TO RELATED APPLICATION

This application is based on and claims priority under 35 USC § 119 to Korean Patent Application Nos. 10-2023-0080329 filed on Jun. 22, 2023, 10-2023-0095656, filed on Jul. 21, 2023, and 10-2023-0172386 filed on Dec. 1, 2023, in the Korean Intellectual Property Office, the disclosures of which are incorporated by reference herein in their entirety.

### BACKGROUND

### 1. Field

The present disclosure is directed to providing a surgical instrument for electrocautery, and more particularly, to a surgical instrument for electrocautery, which may be mounted on a robotic arm or operable manually to be used in laparoscopic surgery or various other surgeries and has improved insulating performance.

### 2. Description of the Related Art

Surgical operations often require the cutting and joining of body tissue, including organs, muscle tissue, connective tissue, and blood vessels. Over the centuries, sharp blades and sutures have been used for cutting and joining. However, during surgical operations, bleeding occurs when body tissue, especially, relatively highly vascularized tissue, is cut. Accordingly, doctors have been in need of surgical instruments and methods to slow or reduce bleeding during surgical operations.

Recently, electrosurgical instruments that use electrical energy to perform certain surgical operations have become available. For example, in surgical instruments, such as graspers, scissors, tweezers, blades, needles, hooks, etc., electrosurgical instruments including one or more electrodes configured to supply electrical energy have been developed. Electrical energy supplied through electrodes may be used to coagulate, join, or cut body tissue of patients. In particular, when electric energy is used, cutting and hemostasis may be achieved at the same time.

Electrosurgical instruments are generally divided into two types: monopolar and bipolar. In a monopolar electrosurgical instrument, electrical energy of a specific polarity is supplied to one or more electrodes of the instrument. Also, electricity of different polarity is electrically connected to a patient. In a bipolar electrosurgical instrument, one or more electrodes are electrically connected to a first polarity electrical energy source, and one or more electrodes are electrically connected to a second polarity electrical energy source opposite the first polarity.

The related art described above is technical information that the inventor possessed for deriving the present disclosure or acquired in the process of deriving the present disclosure and should not be considered as known art disclosed to the general public before filing the application for the present disclosure.

### SUMMARY

The present disclosure provides a surgical instrument for electrocautery having improved insulating performance, which may be mounted on a robotic arm or operable manually for use in laparoscopic surgery or various other surgeries.

Additional aspects will be set forth in portion in the description which follows and, in part, will be apparent from the description, or may be learned by practice of the presented embodiments of the disclosure.

One aspect of the present disclosure provides a surgical instrument for electrocautery, the surgical instrument comprising: an end tool including one or more jaws and configured to be rotatable in two or more directions; a connection part extending in a first direction (an X-axis) and having one end to which the end tool is coupled; a wire having one end connected to the end tool; a shrink tube into which the other end of the wire, opposite to the one end of the wire connected to the end tool, is inserted; and a shaft tube, into which the other end of the shrink tube, opposite to one end of the shrink tube into which the wire is inserted, is inserted, wherein the wire and the shaft tube are separated by the shrink tube, and the wire and the shaft tube are insulated from each other.

In the present disclosure, the other end of the wire is not exposed to the outside of the other end of the shrink tube.

In the present disclosure, a region in which the wire, the shrink tube, and the shaft tube overlap is crimped.

In the present disclosure, in a state in which the wire is inserted into the shrink tube, the shrink tube is heated and contracted to be tightly coupled to the wire.

In the present disclosure, a manipulation part configured to control rotation of the end tool in the two or more directions, and the manipulation part is coupled to the other end of the connection part.

In the present disclosure, the shaft tube connects the wire at the side of the end tool to the wire at the side of the manipulation part.

In the present disclosure, the shaft tube is disposed within the connection part.

In the present disclosure, the wire and the shaft tube are not in direct contact.

In the present disclosure, the wire, the shrink tube, and the shaft tube are each provided in plurality, and the plurality of wires and the plurality of shaft tubes are formed not to directly face each other in any operating state.

In the present disclosure, the surgical instrument further comprises an electric wire connected to the jaw to supply electrical energy to the jaw, wherein a voltage induced in the shaft tube when electric energy flows through the electric wire is blocked from being transmitted to the wire by the shrink tube.

In the present disclosure, the entire wire is formed within the shrink tube.

In the present disclosure, the end tool includes: an end tool hub axially coupled so that an end tool jaw pulley to which the jaw is coupled is rotatable; and a pitch hub axially coupled to the end tool hub by a rotation shaft coupled to one end of the end tool hub, wherein the end tool hub and the pitch hub include an insulator.

In the present disclosure, a whole surface of the end tool hub and the pitch hub includes an insulating material.

In the present disclosure, the end tool includes: an end tool hub axially coupled so that an end tool jaw pulley to which the jaw is coupled is rotatable; a pitch pulley axially coupled to a pitch rotation shaft formed at one end of the end tool hub; a pitch wire wound around the pitch pulley; and a fastening member coupled to the pitch wire to couple the pitch pulley to the pitch wire, wherein the end tool hub and the fastening member are separated by the pitch pulley and the end tool hub and the fastening member are insulated from each other.

In the present disclosure, the pitch wire is formed as a single wire, and the fastening member is fastened to a portion of the pitch wire.

In the present disclosure, the pitch pulley includes an insulator.

In the present disclosure, the pitch pulley includes at least one coupling part fixedly coupling the pitch pulley to the end tool hub.

In the present disclosure, the fastening member is not in direct contact with the end tool hub.

Another aspect of the present disclosure provides a surgical instrument for electrocautery, the surgical instrument comprising: an end tool including one or more jaws and configured to be rotatable in two or more directions; a connection part extending in a first direction (an X-axis) and having one end to which the end tool is coupled; and a wire having one end connected to the end tool, wherein the end tool includes: an end tool hub configured to accommodate at least a portion of the jaw therein; a pitch hub axially coupled to the end tool hub and formed to be rotatable relative to the end tool hub; a relay tube extending to be coupled in a longitudinal direction of the pitch hub and located between the pitch hub and the connection part; and a cover surrounding at least a portion of the end tool including the jaw.

In the present disclosure, the includes an insulating material.

In the present disclosure, the cover includes wrinkles formed in a certain region in which a pitch motion of the end tool takes place.

In the present disclosure, the cover is formed inclined so that a diameter of an end at the side of a proximal end decreases.

In the present disclosure, the pitch hub includes a third pin axially coupled to the end tool hub and a fourth pin formed on one side of the third pin, and the cover includes wrinkles formed in a region adjacent to the fourth pin.

In the present disclosure, one or more cover coupling parts are formed in the relay tube, and one or more recess coupled to the cover coupling parts are formed in the cover.

In the present disclosure, a diameter of a body of the cover is less than a diameter of a body of the relay tube to a certain extent.

In the present disclosure, a shrink tube into which the other end of the wire, opposite to the one end of the wire connected to the end tool, is inserted; and a shaft tube, into which the other end of the shrink tube, opposite to one end of the shrink tube into which the wire is inserted, is inserted, wherein the wire and the shaft tube are separated by the shrink tube, and the wire and the shaft tube are insulated from each other.

In the present disclosure, the other end of the wire is not exposed to the outside of the other end of the shrink tube.

In the present disclosure, a region in which the wire, the shrink tube, and the shaft tube overlap is crimped.

In the present disclosure, the wire and the shaft tube are not in direct contact.

In the present disclosure, the surgical instrument further comprises an electric wire connected to the jaw to supply electrical energy to the jaw, wherein a voltage induced in the shaft tube when electric energy flows through the electric wire is blocked from being transmitted to the wire by the shrink tube.

In the present disclosure, the jaw includes: a first jaw; and a second jaw rotatably coupled to the first jaw, wherein the first jaw and the second jaw each include a tong part, an extension part extending from the tong part in one direction, and a coupling part formed at one end of the extension part, and an end of the cover at the side of the end tool is formed to cover at least a portion of the extension part of the first jaw and the extension part of the second jaw.

In the present disclosure, the first jaw and the second jaw are coupled through a shaft that passes through each coupling part and are coupled so that the extension part of the first jaw and the extension part of the second jaw intersect each other.

In the present disclosure, when the first jaw and the second jaw are closed, the extension part of the first jaw and the extension part of the second jaw intersect in an X-shape.

In the present disclosure, the surgical instrument further comprises a jaw rotation shaft inserted through the first jaw and the second jaw to be a rotation center of the first jaw and the second jaw; and an intersection point at which the extension part of the first jaw and the extension part of the second jaw intersect in an X-shape when the first jaw and second jaw are closed, wherein the intersection point is apart from the jaw rotation shaft.

Another aspect of the present disclosure provides a surgical instrument for electrocautery, the surgical instrument comprising: an end tool including one or more jaws and configured to be rotatable in two or more directions; a connection part extending in a first direction (an X-axis) and having one end to which the end tool is coupled; and a wire having one end connected to the end tool, wherein the jaw includes: a first jaw; and a second jaw rotatably coupled to the first jaw, wherein the first jaw and the second jaw each include a tong part, an extension part extending from the tong part in one direction, and a coupling part formed at one end of the extension part, and the first jaw and the second jaw are coupled through a shaft that passes through each coupling part and are coupled so that the extension part of the first jaw and the extension part of the second jaw intersect each other.

In the present disclosure, when the first jaw and the second jaw are closed, the extension part of the first jaw and the extension part of the second jaw intersect in an X-shape.

In the present disclosure, the surgical instrument further comprises a jaw rotation shaft inserted through the first jaw and the second jaw to be a rotation center of the first jaw and the second jaw; and an intersection point at which the extension part of the first jaw and the extension part of the second jaw intersect in an X-shape when the first jaw and second jaw are closed, wherein the intersection point is apart from the jaw rotation shaft.

In the present disclosure, the surgical instrument further comprises a cover surrounding an exterior of a component located between the jaw and the connection part, wherein an inlet part of the cover is located adjacent to the intersection point at which the extension part of the first jaw and the extension part of the second jaw intersect.

In the present disclosure, the cover includes an insulating material.

In the present disclosure, the cover includes wrinkles formed in a certain region in which a pitch motion of the end tool takes place.

In the present disclosure, the cover is formed inclined so that a diameter of an end at the side of a proximal end decreases.

In the present disclosure, the tong part and the extension part are formed at a certain angle, and when the first jaw and the second jaw are closed, based on one surface in which the tong parts contact each other, a portion of the extension part adjacent to the coupling part of the second jaw and the tong part of the first jaw are located on one side of the one surface and a portion of the extension part adjacent to the coupling part of the first jaw and the tong part of the second jaw are located on the other side of the one surface.

In the present disclosure, the surgical instrument further comprises a first jaw pulley coupled to the first jaw and configured to be rotatable about a first shaft; and a second jaw pulley coupled to the second jaw and formed to be rotatable about the first shaft, wherein the first shaft is a jaw pulley rotation shaft inserted through the first jaw pulley and the second jaw pulley to be a rotation center of the first jaw pulley and the second jaw pulley, and when the first jaw pulley and the second jaw pulley rotate about the jaw pulley rotation shaft, the jaw rotation shaft moves relative to the jaw pulley rotation shaft.

In the present disclosure, a movement coupling hole is formed in the first jaw or the second jaw, a shaft coupling part is formed in the first jaw pulley or the second jaw pulley, and the shaft coupling part is formed to be movable to a certain degree within the movement coupling hole in a state in which the shaft coupling part is fitted into the movement coupling hole,

In the present disclosure, when the first jaw and the second jaw are closed, the jaw rotation shaft moves toward a distal end of the end tool, and when the first jaw and the second jaw are open, the jaw rotation shaft moves toward a proximal end of the end tool.

In the present disclosure, the surgical instrument further comprises a shrink tube into which the other end of the wire, opposite to the one end of the wire connected to the end tool, is inserted; and a shaft tube, into which the other end of the shrink tube, opposite to one end of the shrink tube into which the wire is inserted, is inserted, wherein the wire and the shaft tube are separated by the shrink tube, and the wire and the shaft tube are insulated from each other.

In the present disclosure, the other end of the wire is not exposed to the outside of the other end of the shrink tube.

In the present disclosure, a region in which the wire, the shrink tube, and the shaft tube overlap is crimped.

In the present disclosure, the wire and the shaft tube are not in direct contact.

Another aspect of the present disclosure provides a surgical instrument for electrocautery, the surgical instrument comprising: an end tool including one or more jaws and configured to be rotatable in two or more directions; a connection part extending in a first direction (an X-axis) and having one end to which the end tool is coupled; and a wire having one end connected to the end tool; wherein the wire is an entirely coated electric wire, transmits power to the end tool, and transmits electrical energy to the jaw through the wire.

In the present disclosure, the surgical instrument further comprises a manipulation part configured to control rotation of the end tool in the two or more directions, wherein the manipulation part is coupled to the other end of the connection part.

In the present disclosure, the wire is a power transmission part connected to the manipulation part to transmit rotation of the manipulation part to the jaw.

In the present disclosure, the jaw includes a conductor.

In the present disclosure, the wire includes a fastening region, which is a portion from which the coating of one end is removed, and the fastening region is in contact with a portion of the jaw to transmit electrical energy to the jaw through the wire.

In the present disclosure, the wire is located within the end tool and the connection part, and a portion other than the fastening region is covered.

In the present disclosure, the wire is formed as a single electric wire, and a fastening member is coupled to a portion in which coating is removed at a middle point of the single wire.

In the present disclosure, the fastening member is crimped and fixed to the wire, and a coating electric wire is formed on both wires based on the fastening member.

In the present disclosure, the end tool includes an end tool jaw pulley on which at least a portion of the jaw is accommodated, and the end tool jaw pulley includes an insulator.

In the present disclosure, the wire is wound around the end tool jaw pulley and extends toward the connection part.

Another aspect of the present disclosure provides a method of manufacturing a surgical instrument for electrocautery, the method comprising: inserting one end of the wire into a shrink tube; tightly coupling the shrink tube to the wire by heating the shrink tube; inserting one end of the shrink tube, which is opposite to the other end into which the wire is inserted, into the shaft tube; and crimping the wire, the shrink tube, and the shaft tube together.

In the present disclosure, the inserting of one end of the wire into the shrink tube includes inserting one end of the wire so that the one end of the wire does not protrude to the outside of the one end of the shrink tube.

In the present disclosure, the crimping of the wire, the shrink tube, and the shaft tube together includes crimping a region in which the wire, the shrink tube, and the shaft tube overlap.

In the present disclosure, the method further comprises connecting the other end of the wire to an end tool; and connecting one end of the shaft tube to a wire at the side of a manipulation part.

In the present disclosure, the wire and the shaft tube are not in direct contact.

In the present disclosure, the wire, the shrink tube, and the shaft tube are each provided in plurality, and the plurality of wires and the plurality of shaft tubes are formed not to directly face each other in any operating state.

In the present disclosure, an electric wire connected to a jaw to supply electrical energy to the jaw is further included, a voltage induced in the shaft tube when electric energy flows through the electric wire is blocked from being transmitted to the wire by the shrink tube.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1A is a conceptual diagram of a pitch motion of a surgical instrument of a related art, and FIG. 1B is a conceptual diagram of a yaw motion;

FIG. 1C is a conceptual diagram of a pitch motion of a surgical instrument of another related art, and FIG. 1D is a conceptual diagram of a yaw motion;

FIG. 1E is a conceptual diagram of a pitch motion of a surgical instrument according to the present disclosure, and FIG. 1F is a conceptual diagram of a yaw motion;

FIG. 2 is a perspective view illustrating a surgical instrument for electrocautery according to a first embodiment of the present disclosure;

FIG. 3 is a side view of the surgical instrument for electrocautery of FIG. 2;

FIGS. 4 and 5 are perspective views illustrating an end tool of the surgical instrument for electrocautery of FIG. 2;

FIG. 6 is an enlarged perspective view illustrating an end tool hub of the surgical instrument for electrocautery of FIG. 4;

FIG. 7 is an enlarged perspective view illustrating another embodiment of the end tool hub of FIG. 6;

FIG. 8 is a front view illustrating a guide member of FIG. 7;

FIGS. 9 and 10 are plan views illustrating an end tool of the surgical instrument for electrocautery of FIG. 2;

FIGS. 11 and 12 are perspective views illustrating a manipulation part of the surgical instrument for electrocautery of FIG. 2;

FIG. 13 is a diagram schematically illustrating only a configuration of a pulley and a wire constituting a joint of the surgical instrument for electrocautery shown in FIG. 2;

FIGS. 14 to 16 are diagrams schematically illustrating a wire connection configuration within a connection part of the surgical instrument for electrocautery shown in FIG. 2;

FIG. 17 is a diagram illustrating a modified example of the embodiment illustrated in FIG. 16;

FIGS. 18 and 19 are perspective views illustrating an end tool of a surgical instrument for electrocautery according to a second embodiment of the present disclosure;

FIG. 20 is a perspective view illustrating an end tool hub and a pitch hub of a surgical instrument for electrocautery according to a third embodiment of the present disclosure;

FIGS. 21A and 21B are perspective views illustrating an end tool of a surgical instrument for electrocautery according to a fourth embodiment of the present disclosure, FIG. 21C is a perspective view illustrating an insulating pulley of the end tool shown in FIGS. 21A and 21B, and FIG. 21D is a perspective view illustrating an end tool hub shown in FIGS. 21A and 21B;

FIG. 22 is a diagram illustrating a jaw pulley and a wire connection configuration in an end tool of a surgical instrument for electrocautery according to a fifth embodiment of the present disclosure;

FIG. 23 is a perspective view illustrating a surgical instrument for electrocautery according to a sixth embodiment of the present disclosure;

FIG. 24 is a side view of the surgical instrument for electrocautery of FIG. 23;

FIGS. 25 to 27 are perspective views illustrating an end tool of the surgical instrument for electrocautery of FIG. 23;

FIG. 28 is an exploded perspective view illustrating an end tool hub, a pitch hub, a relay tube, and a shaft shown in FIG. 26;

FIG. 29 is an enlarged perspective view illustrating the end tool hub of the surgical instrument for electrocautery of FIG. 26;

FIG. 30 is an enlarged perspective view illustrating the relay tube of the surgical instrument for electrocautery of FIG. 26;

FIG. 31 is an exploded perspective view illustrating a jaw coupling structure of the surgical instrument for electrocautery of FIG. 26;

FIG. 32 is a plan view illustrating a first jaw of FIG. 31;

FIG. 33 is a plan view illustrating a second jaw of FIG. 31;

FIGS. 34A and 34B are plan views illustrating coupling of a first jaw and a second jaw of FIG. 29;

FIGS. 35A and 35B are diagrams illustrating a jaw having an existing structure of a surgical instrument for electrocautery;

FIGS. 36A and 36B are diagrams illustrating a jaw of a surgical instrument for electrocautery of FIG. 23;

FIGS. 37A and 37B are views illustrating a cover of the surgical instrument for electrocautery of FIG. 23;

FIGS. 38A and 38B are diagrams illustrating an end tool of the surgical instrument for electrocautery of FIG. 23;

FIGS. 39 and 40 are perspective views illustrating a yaw motion of the surgical instrument for electrocautery of FIG. 23;

FIGS. 41 and 42 are perspective views illustrating a pitch motion of the surgical instrument for electrocautery of FIG. 23;

FIG. 43 is a perspective view illustrating a yaw-pitch motion of the surgical instrument for electrocautery of FIG. 23;

FIG. 44 is a perspective view illustrating a surgical instrument for electrocautery according to a seventh embodiment of the present disclosure;

FIG. 45 is a side view illustrating the surgical instrument for electrocautery of FIG. 44; and

FIGS. 46 and 47 are perspective views illustrating an end tool of the surgical instrument for electrocautery of FIG. 44.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions, such as “at least one of,” when preceding a list of elements, modify the entire list of elements and do not modify the individual elements of the list.

While the present disclosure is susceptible to various modifications and alternative forms, specific embodiments thereof are shown by way of example in the drawings and will herein be described in detail. However, it should be understood that there is no intent to limit the present disclosure to the particular forms disclosed herein, rather, the present disclosure should be construed to cover various modifications, equivalents, and alternatives of embodiments of the present disclosure. In describing the present disclosure, detailed description of known related arts will be omitted when it is determined that the gist of the present disclosure may be unnecessarily obscured

Although terms such as “first,” “second,” and the like may be used to describe various components, such components should not be limited to the above terms The terms are only used to distinguish one component from another.

The terms used herein are for the purpose of describing particular embodiments only and are not intended to be limiting to the present disclosure. Singular forms are intended to include plural forms as well, unless the context clearly indicates otherwise. In the present application, it will be further understood that the terms “comprise,” “comprising,” “include,” and/or “including”, when used in this specification, specify the presence of stated features, integers, steps, operations, elements, components and/or groups thereof but do not preclude the presence or addition of one or more other features, integers, steps, operations, elements, components and/or groups thereof.

Hereinafter, the embodiments of the present disclosure will be described below in detail with reference to the accompanying drawings, and when the embodiments of the present disclosure are described with reference to the drawings, the same or corresponding components are given the same reference numerals, and repetitive descriptions thereof will be omitted.

Further, in describing the various embodiments of the present disclosure, it is to be understood that each embodiment is not intended to be interpreted or implemented independently, and that the technical ideas described in each embodiment may be interpreted or implemented in combination with other embodiments described separately.

In a surgical instrument for electrocautery according to the present disclosure, when a manipulation part is rotated in one direction for at least any one of pitch, yaw, and actuation motions, an end tool is rotated in intuitively the same direction as a direction in which the manipulation part is moved.

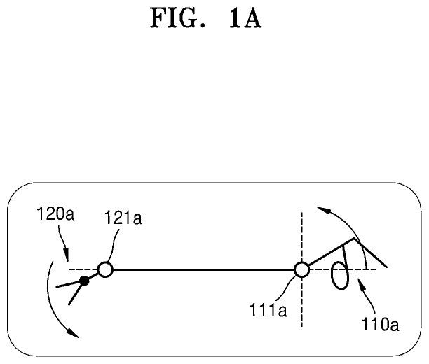

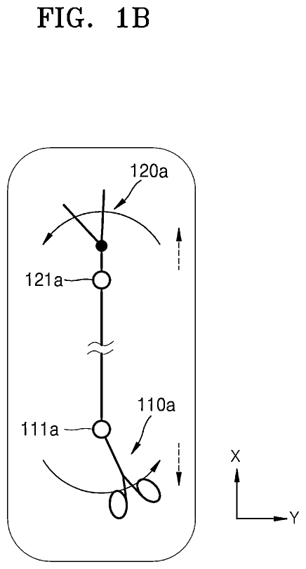

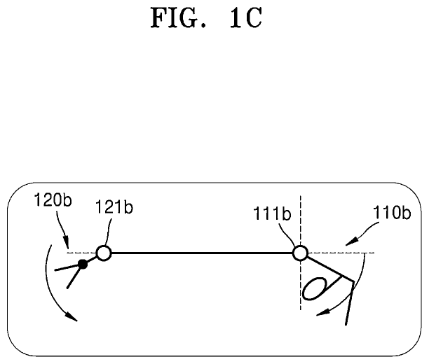

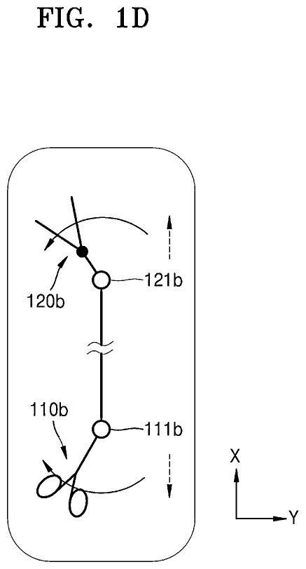

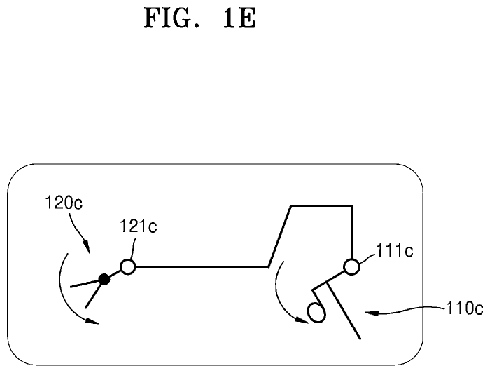

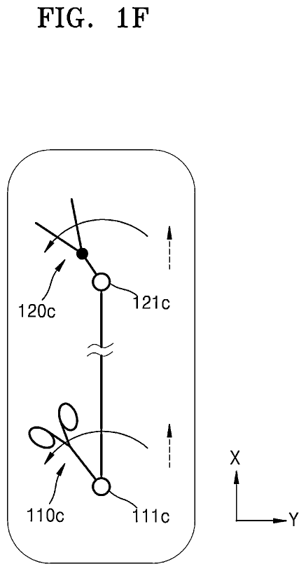

FIG. 1A is a conceptual diagram of a pitch motion of a conventional surgical instrument, and FIG. 1B is a conceptual diagram of a yaw motion thereof.

Referring to FIG. 1A, in performing a pitch motion of a conventional surgical instrument, in a state in which an end tool 120a is formed in front of a rotation center 121a of the end tool, and a manipulation part 110a is formed at the rear of a rotation center 111a of the manipulation part, when the manipulation part 110a is rotated in a clockwise direction, the end tool 120a is also rotated in the clockwise direction, and when the manipulation part 120a is rotated in a counterclockwise direction, the end tool 120a is also rotated in the counterclockwise direction. Referring to FIG. 1B, in performing a yaw motion of the conventional surgical instrument, in a state in which the end tool 120a is formed in front of the rotation center 121a of the end tool, and the manipulation part 110a is formed at the rear of the rotation center 111a of the manipulation part, when the manipulation part 110a is rotated in the clockwise direction, the end tool 120a is also rotated in the clockwise direction, and when the manipulation part 120a is rotated in the counterclockwise direction, the end tool 120a is also rotated in the counterclockwise direction. In this case, in view of left and right directions of a user, when the user moves the manipulation part 110a to the left, the end tool 120a is moved to the right, and when the user moves the manipulation part 110a to the right, the end tool 120a is moved to the left. As a result, a manipulation direction of the user and an operation direction of the end tool are opposite to each other, which may cause the user to make a mistake, and user's manipulation may not be easy.

FIG. 1C is a conceptual diagram of a pitch motion of another conventional surgical instrument, and FIG. 1D is a conceptual diagram of a yaw motion thereof.

Referring to FIG. 1C, in the conventional surgical instrument, which is partially formed in a mirror symmetrical shape, in performing a pitch motion, in a state in which an end tool 120b is formed in front of a rotation center 121b of the end tool, and a manipulation part 110b is formed at the rear of a rotation center 111b of the manipulation part, when the manipulation part 110b is rotated in the clockwise direction, the end tool 120b is rotated in the counterclockwise direction, and when the manipulation part 110b is rotated in the counterclockwise direction, the end tool 120b is rotated in the clockwise direction. In this case, in view of rotation directions of the manipulation part and the end tool, a rotation direction in which the user rotates the manipulation part 110b and a rotation direction of the end tool 120b according thereto are opposite to each other. As a result, the user may be confused with the manipulation direction, and as the operation of a joint is not intuitive, the user may make an error. Further, referring to FIG. 1D, in performing a yaw motion, in a state in which the end tool 120b is formed in front of the rotation center 121b of the end tool, and the manipulation part 110b is formed at the rear of the rotation center 111b of the manipulation part, when the manipulation part 110b is rotated in the clockwise direction, the end tool 120b is rotated in the counterclockwise direction, and when the manipulation part 110b is rotated in the counterclockwise direction, the end tool 120b is rotated in the clockwise direction. In this case, in view of rotation directions of the manipulation part and the end tool, a rotation direction in which the user rotates the manipulation part 110b and a rotation direction of the end tool 120b according thereto are opposite to each other. As a result, the user may be confused with the manipulation direction, and as the operation of the joint is not intuitive, the user may make an error. In the user's pitch or yaw manipulation of the conventional surgical instrument, the user's manipulation direction and the end tool's operation direction do not match each other in view of one of the rotation direction and the left and right directions. This is because the configurations of the end tool and the manipulation part are different from each other in the joint configuration of the conventional surgical instrument. That is, this is because the manipulation part is formed at the rear of the rotation center of the manipulation part, while the end tool is formed in front of the rotation center of the end tool. In order to address the above problems, in a surgical instrument according to an embodiment of the present disclosure, which is illustrated in FIGS. 1E and 1F, an end tool 120c is formed in front of a rotation center 121c of the end tool and a manipulation part 110c is also formed in front of a rotation center 111c of the manipulation part, so that the operations of the manipulation part 110c and the end tool 120c are intuitively matched with each other. In other words, unlike existing examples such as those shown in FIGS. 1A, 1B, 1C, and 1D, in which the manipulation part is close to a user with respect to the joint thereof (that is, away from the end tool), the surgical instrument according to an embodiment of the present disclosure, which is illustrated in FIGS. 1E and 1F, is formed such that at least a portion of the manipulation part is closer (than a joint thereof) to the end tool with respect to the joint thereof at any one moment or more in a manipulation process.

In other words, in the conventional surgical instrument as illustrated in FIGS. 1A, 1B, 1C, and 1D, the manipulation part is formed at the rear of the rotation center thereof, while the end tool is located in front of the rotation center thereof, and thus the end tool is moved at a front side thereof with a rear side fixed through a motion of the manipulation part that is moved at a rear side thereof with a front side thereof fixed, which is not an intuitively matching structure. Accordingly, a mismatch may occur between the manipulation of the manipulation part and the motion of the end tool in view of the left and right directions or in view of the rotation direction, which may cause confusion to the user, and the manipulation of the manipulation part may be difficult to perform intuitively and quickly and may cause mistakes. In contrast, in the surgical instrument according to an embodiment of the present disclosure, since both the end tool and the manipulation part are moved with respect to the rotation center formed at the rear side thereof, it may be said that the motions are intuitively matched with each other in terms of structure. In other words, moving portions of the manipulation part are moved with respect to the rotation center formed at the rear side thereof just as moving portions of the end tool are moved with respect to the rotation center formed at the rear side thereof, and thus it may be said that the motions are intuitively matched with each other in terms of structure. This allows the user to intuitively and quickly perform a control in a direction toward the end tool, and a possibility of making a mistake may be significantly reduced. Hereinafter, a detailed mechanism enabling the above-described function will be described below.

### <A Surgical Instrument for Electrocautery>

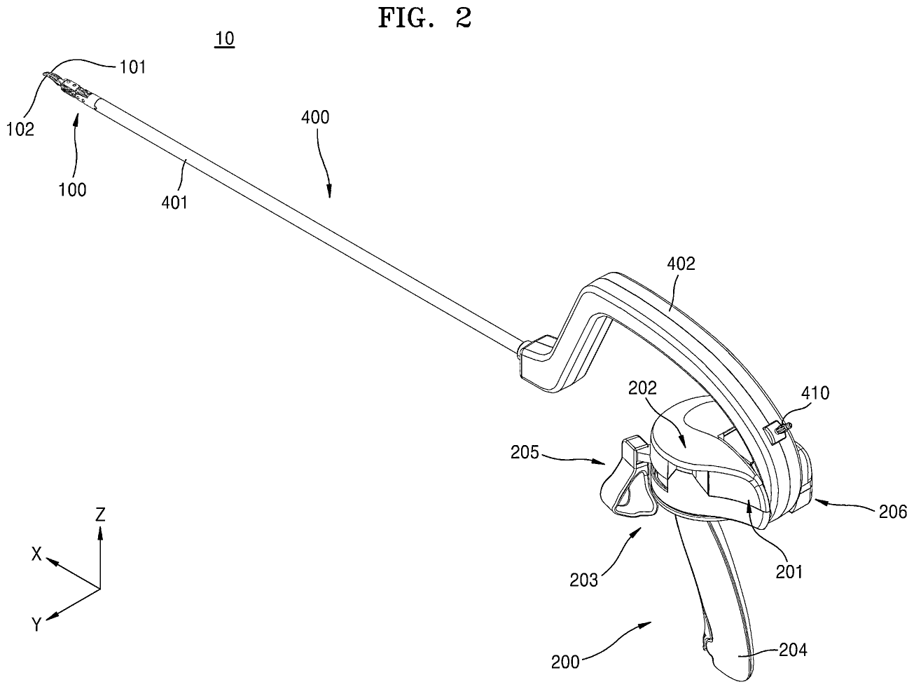

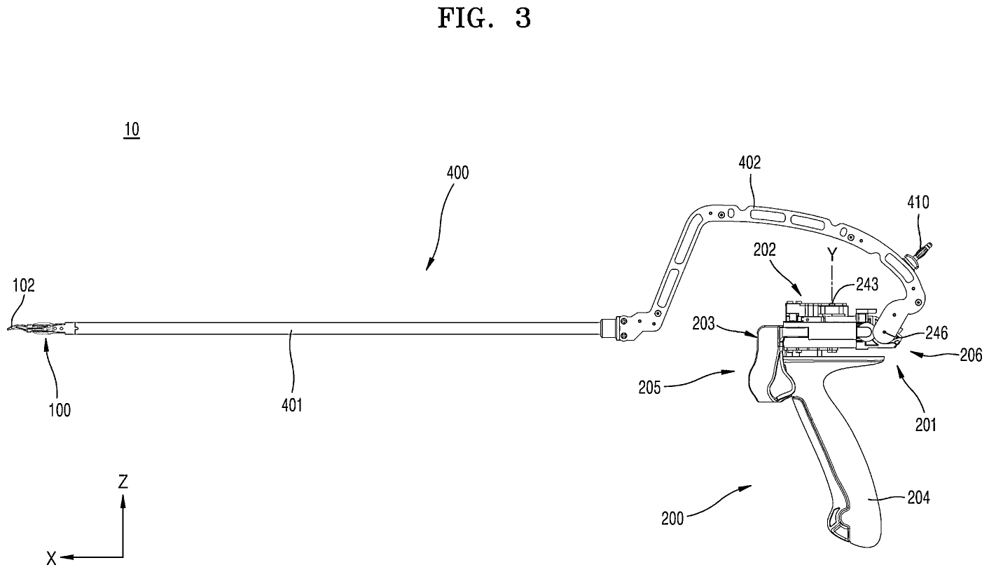

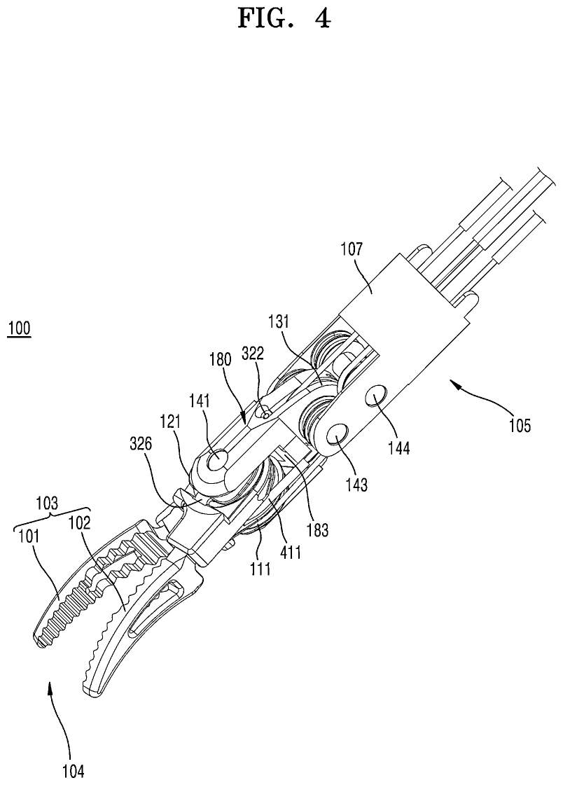

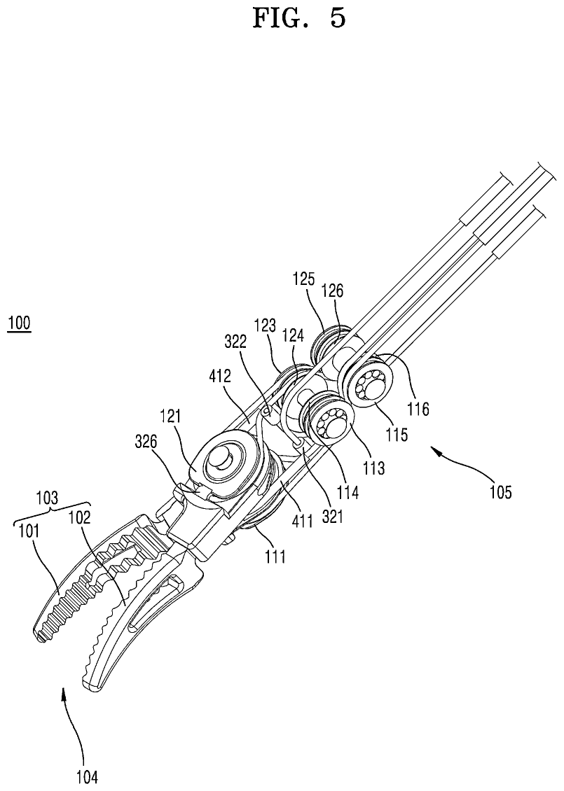

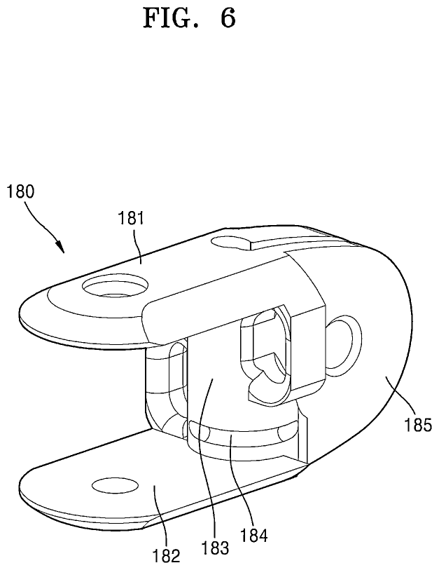

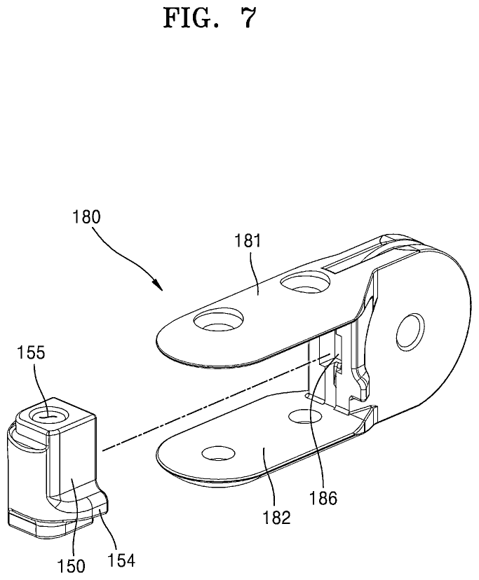

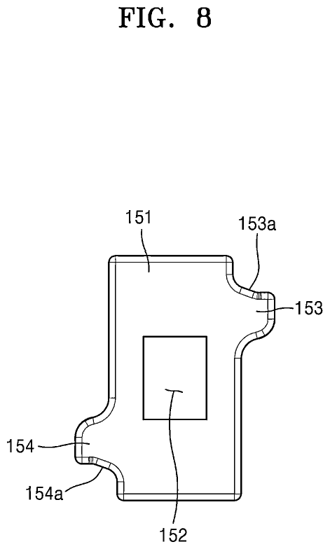

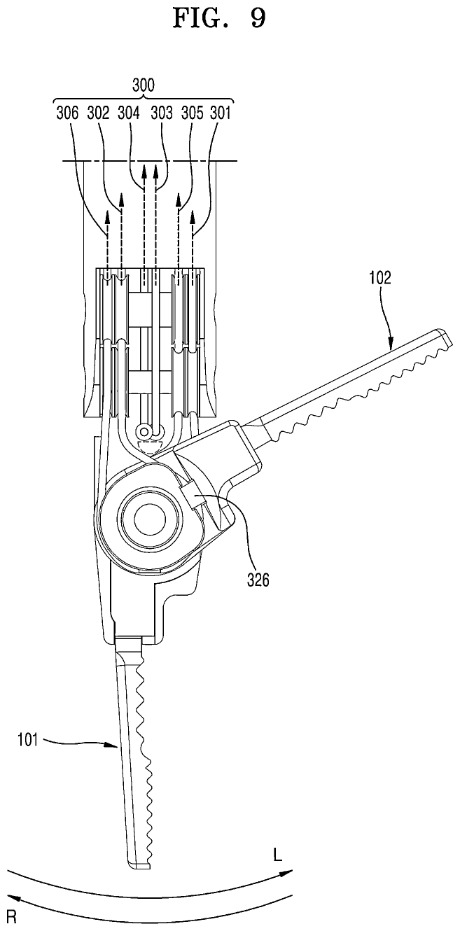

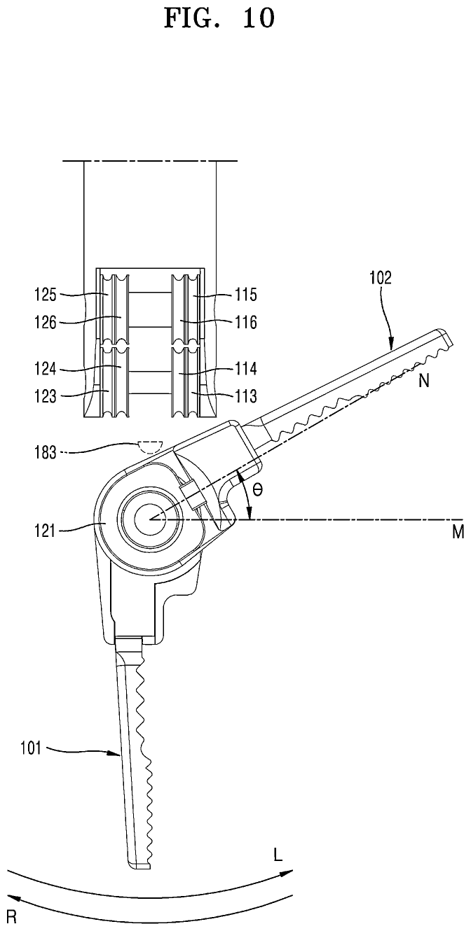

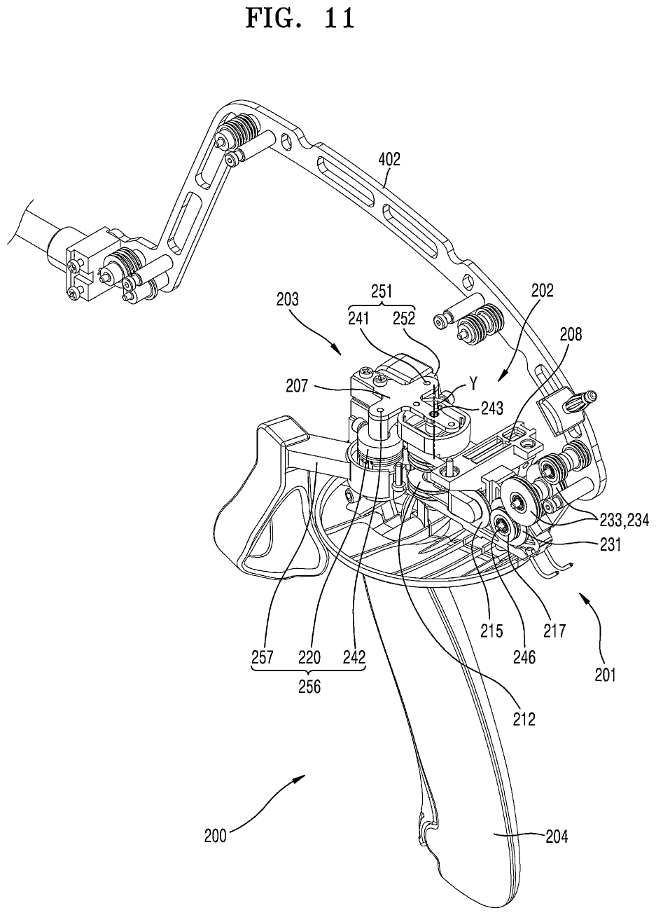

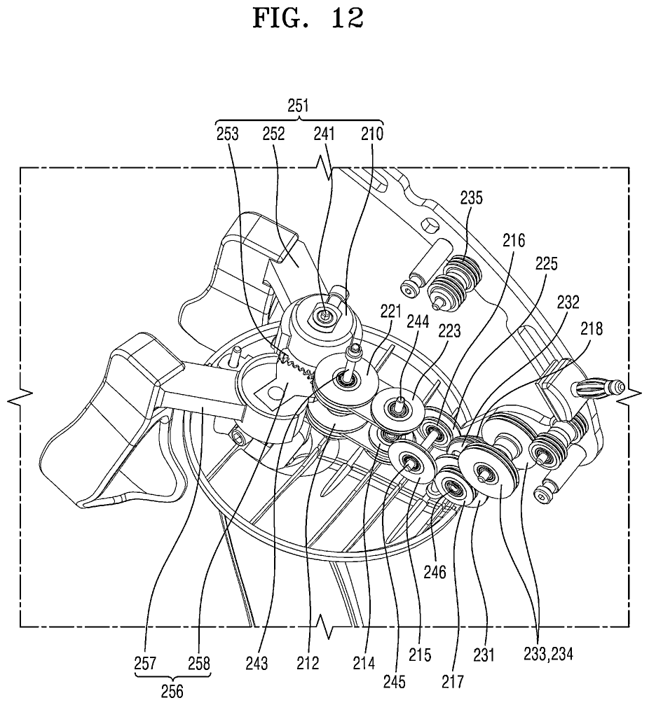

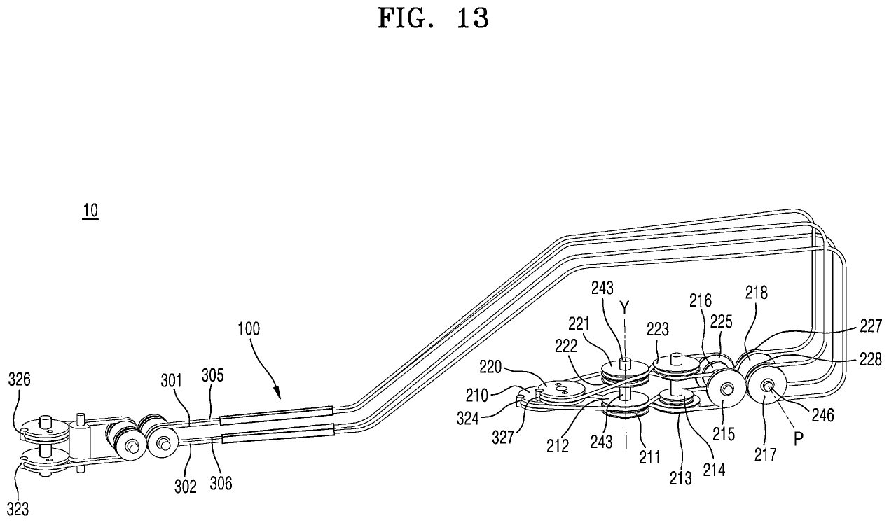

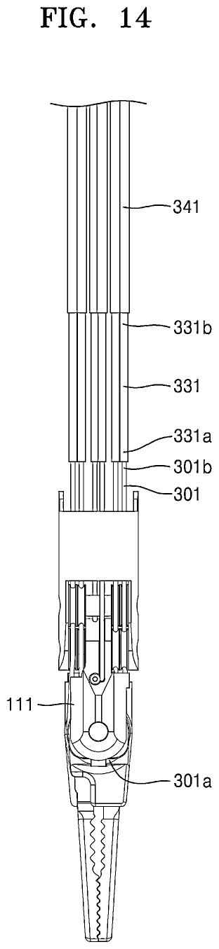

FIG. 2 is a perspective view illustrating a surgical instrument for electrocautery according to a first embodiment of the present disclosure; FIG. 3 is a side view of the surgical instrument for electrocautery of FIG. 2; FIGS. 4 and 5 are perspective views illustrating an end tool of the surgical instrument for electrocautery of FIG. 2; FIG. 6 is an enlarged perspective view illustrating an end tool hub of the surgical instrument for electrocautery of FIG. 4; FIG. 7 is an enlarged perspective view illustrating another embodiment of the end tool hub of FIG. 6; FIG. 8 is a front view illustrating a guide member of FIG. 7; FIGS. 9 and 10 are plan views illustrating an end tool of the surgical instrument for electrocautery of FIG. 2; FIGS. 11 and 12 are perspective views illustrating a manipulation part of the surgical instrument for electrocautery of FIG. 2; FIG. 13 is a diagram schematically illustrating only a configuration of a pulley and a wire constituting a joint of the surgical instrument for electrocautery shown in FIG. 2; FIGS. 14 to 16 are diagrams schematically illustrating a wire connection configuration within a connection part of the surgical instrument for electrocautery shown in FIG. 2.

First, referring to FIGS. 2 and 3, a surgical instrument for electrocautery 10 according to a first embodiment of the present disclosure includes an end tool 100, a manipulation part 200, a power transmission part 300, and a connection part 400.

Here, the connection part 400 is formed in the shape of a hollow shaft, and one or more wires and electric wires may be accommodated therein. The manipulation part 200 is coupled to one end portion of the connection part 400, the end tool 100 is coupled to the other end portion thereof, and the connection part 400 may serve to connect the manipulation part 200 and the end tool 100. Here, the connection part 400 of the surgical instrument for electrocautery 10 according to the first embodiment of the present disclosure includes a straight part 401 and a bent part 402, wherein the straight part 401 is formed at a side coupled to the end tool 100, and the bent part 402 is formed at a side to which the manipulation part 200 is coupled. As such, since the end portion of the connection part 400 at the side of the manipulation part 200 is formed to be bent, a pitch manipulation part 201, a yaw manipulation part 202, and an actuation manipulation part 203 may be formed along an extension line of the end tool 100 or adjacent to the extension line. From another perspective, it may be said that the pitch manipulation part 201 and the yaw manipulation part 202 are at least partially accommodated in a concave portion formed by the bent part 402. Due to the above-described shape of the bent part 402, the shapes and motions of the manipulation part 200 and the end tool 100 may be further intuitively matched with each other.

Meanwhile, a plane on which the bent part 402 is formed may be substantially the same plane as a pitch plane, that is, an XZ plane of FIG. 2. As such, as the bent part 402 is formed on substantially the same plane as the XZ plane, interference with the manipulation part may be reduced. Of course, for intuitive motions of the end tool and the manipulation part, any form other than the XZ plane may be possible.

Meanwhile, a connector 410 may be formed in the bent part 402. The connector 410 may be connected to an external power source (not shown), and the connector 410 may also be connected to a jaw 103 through electric wires 411 and 412 to transfer electrical energy supplied from the external power source (not shown) to the jaw 103. Here, the drawing shows a monopolar type with one electrode formed in the connector 410, but the present disclosure is not limited thereto, and a bipolar type electrode may be formed in the connector.

The manipulation part 200 is formed at the one end portion of the connection part 400 and provided as an interface to be directly controlled by a medical doctor, for example, a tongs shape, a stick shape, a lever shape, or the like, and when the medical doctor controls the manipulation part 200, the end tool 100, which is connected to the interface and inserted into the body of a surgical patient, performs a certain motion, thereby performing surgery. Here, the manipulation part 200 is illustrated in FIG. 2 as being formed in a handle shape that is rotatable while the finger is inserted therein, the concept of the present disclosure is not limited thereto, and various types of manipulation parts that are connected to the end tool 100 and manipulate the end tool 100 may be possible.

The end tool 100 is formed on the other end portion of the connection part 400, and performs necessary motions for surgery by being inserted into a surgical site. In an example of the end tool 100, as illustrated in FIG. 2, a pair of jaws 103 for performing a grip motion may be used. However, the concept of the present disclosure is not limited thereto, and various devices for performing surgery may be used as the end tool 100. For example, a configuration of a cantilever cautery may also be used as the end tool. The end tool 100 is connected to the manipulation part 200 by the power transmission part 300, and receives a driving force of the manipulation part 200 through the power transmission part 300 to perform a motion necessary for surgery, such as gripping, cutting, suturing, or the like.

Here, the end tool 100 of the surgical instrument for electrocautery 10 according to the first embodiment of the present disclosure is formed to be rotatable in at least one direction, for example, the end tool 100 may perform a pitch motion around a Y-axis of FIG. 2 and simultaneously perform a yaw motion and an actuation motion around a Z-axis of FIG. 2.

Here, each of the pitch, yaw, and actuation motions used in the present disclosure are defined as follows.

First, the pitch motion means a motion of the end tool 100 rotating in a vertical direction with respect to an extension direction of the connection part 400 (an X-axis direction of FIG. 2), that is, a motion rotating around the Y-axis of FIG. 2. In other words, the pitch motion means a motion of the end tool 100, which is formed to extend from the connection part 400 in the extension direction of the connection part 400 (the X-axis direction of FIG. 2), rotating vertically around the Y-axis with respect to the connection part 400.

Next, the yaw motion means a motion of the end tool 100 rotating in the left and right directions, that is, a motion rotating around the Z-axis of FIG. 2, with respect to the extension direction of the connection part 400 (the X-axis direction of FIG. 2). In other words, the yaw motion means a motion of the end tool 100, which extends from the connection part 400 in the extension direction of the connection part 400 (the X-axis direction of FIG. 2), rotating horizontally around the Z-axis with respect to the connection part 400. That is, the yaw motion means a motion of the two jaws 103, which are formed on the end tool 100, rotating around the Z-axis in the same direction.

Meanwhile, the actuation motion may mean a motion of the end tool 100 rotating around the same shaft of rotation as that of the yaw motion, while the two jaws 103 rotating in the opposite directions so as to be closed or opened. That is, the actuation motion means rotating motions of the two jaws 103, which are formed on the end tool 100, in the opposite directions around the Z-axis.

The power transmission part 300 may connect the manipulation part 200 to the end tool 100, transmit the driving force of the manipulation part 200 to the end tool 100, and include a plurality of wires, pulleys, links, sections, gears, or the like.

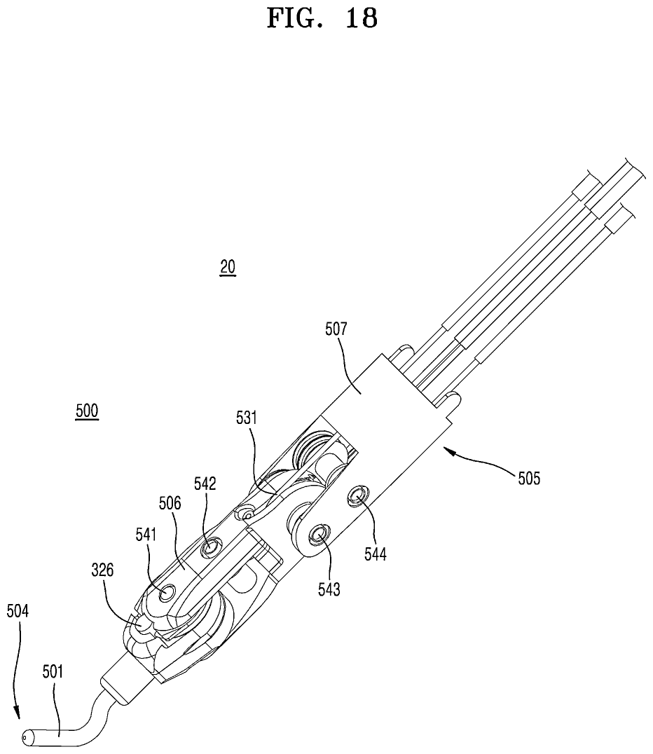

Here, the drawing shows a monopolar dissector as an example of a surgical instrument for electrocautery, but the present disclosure is not limited thereto and may be applied to various monopolar surgical instruments for electrocautery, such as monopolar scissors and monopolar hooks, and monopolar spatulas. Furthermore, the present disclosure may also be applied to various bipolar electrocautery surgical instruments for electrocautery, such as bipolar dissectors and bipolar forceps. The monopolar hook will be described in detail below with reference to FIGS. 18 and 19.

The end tool 100, the manipulation part 200, and the power transmission part 300 of the surgical instrument for electrocautery 10 of FIG. 2 will be described in detail later.

### (Intuitive Driving)

Hereinafter, intuitive driving of the surgical instrument for electrocautery 10 of the present disclosure will be described.

First, while holding a first handle 204 with the palm of the hand, the user may rotate a first handle 204 around the Y-axis (i.e., a rotation shaft 246) to perform a pitch motion, and rotate the first handle 204 around the Z-axis (i.e., a rotation shaft 243) to perform a yaw motion. In addition, the user may perform an actuation motion by manipulating the actuation manipulation part 203 while inserting the thumb and the index finger into a first actuation extension part 252 and/or a second actuation extension part 257 in the form of a hand ring formed at one end portion of the actuation manipulation part 203.

Here, in the surgical instrument for electrocautery 10 according to the first embodiment of the present disclosure, when the manipulation part 200 is rotated in one direction with respect to the connection part 400, the end tool 100 is rotated in a direction that is intuitively the same as a manipulation direction of the manipulation part 200. In other words, when the first handle 204 of the manipulation part 200 is rotated in one direction, the end tool 100 is also rotated in a direction intuitively the same as the one direction, so that a pitch motion or a yaw motion is performed. Here, the phrase “intuitively the same direction” may be further explained as meaning that a direction of movement of the user's finger gripping the manipulation part 200 and a direction of movement of a distal end of the end tool 100 form substantially the same direction. Of course, “the same direction” as used herein may not be a perfectly matching direction on a three-dimensional coordinate, and may be understood to be equivalent to the extent that, for example, when the user's finger moves to the left, the distal end of the end tool 100 is moved to the left, and when the user's finger moves down, the end portion of the end tool 100 is moved down.

In addition, to this end, in the surgical instrument for electrocautery 10 according to the first embodiment of the present disclosure, the manipulation part 200 and the end tool 100 are formed in the same direction with respect to a plane perpendicular to the extension axis (X-axis) of the connection part 400. That is, when viewed based on a YZ plane of FIG. 2, the manipulation part 200 is formed to extend in a positive (+) X-axis direction, and the end tool 100 is also formed to extend in the positive (+) X-axis direction. In other words, it may be said that a formation direction of the end tool 100 on one end portion of the connection part 400 is the same as a formation direction of the manipulation part 200 on the other end portion of the connection part 400 on the basis of the YZ plane. Further, in other words, it may be said that the manipulation part 200 may be formed in a direction away from the body of a user holding the manipulation part 200, that is, in a direction in which the end tool 100 is formed. That is, in the parts such as the first handle 204, a first actuation manipulation part 251, a second actuation manipulation part 256, and the like, which are moved by the user's grip for actuation motion, yaw motion, and pitch motions, a corresponding portion that is moved for the motion is formed to extend in the positive (+) X-axis direction from the rotation center of a corresponding joint for the motion. In this manner, the manipulation part 200 may be configured in the same manner as the end tool 100 in which each moving portion is formed to extend in the positive (+) X-axis direction from the rotation center of a corresponding joint for the motion, and as described with reference to FIG. 1, the manipulation direction of the user may be identical to the operation direction of the end tool from the viewpoint of the rotation directions and the left and right directions. As a result, intuitively the same manipulation may be achieved.

In detail, in the case of the conventional surgical instrument, a direction in which a user manipulates the manipulation part is different from a direction in which the end tool is actually operated, that is, intuitively different from the direction in which the end tool is actually operated, and thus, a surgical operator may not easily intuitively manipulate the surgical instrument and may spend a long time to learn a skill of operating the end tool in desired directions, and in some cases, malfunctions may occur, which may cause damage to patients.

In order to address such problems, the surgical instrument for electrocautery 10 according to the first embodiment of the present disclosure is configured such that the manipulation direction of the manipulation part 200 and the operation direction of the end tool 100 are intuitively identical to each other. To this end, the manipulation part 200 is configured like the end tool 100, that is, in the manipulation part 200, portions that are actually moved for actuation, yaw, and pitch motions extend respectively from rotation centers of corresponding joints in the positive (+) X-axis direction.

Hereinafter, the end tool 100, the manipulation part 200, the power transmission part 300, and the like of the surgical instrument for electrocautery 10 of FIG. 2 will be described in more detail.

### (Power Transmission Part)

Hereinafter, the power transmission part 300 of the surgical instrument for electrocautery 10 of FIG. 2 will be described in more detail.

Referring to FIGS. 2 to 13, and the like, the power transmission part 300 of the surgical instrument for electrocautery 10 according to an embodiment of the present disclosure may include a wire 301, a wire 302, a wire 303, a wire 304, a wire 305, a wire 306.

Here, the wires 301 and 305 may be paired to serve as first jaw wires. The wires 302 and 306 may be paired to serve as second jaw wires. Here, the components encompassing the wires 301 and 305, which are first jaw wires, and the wires 302 and 306, which are second jaw wires, may be referred to as jaw wires. In addition, the wires 303 and 304 may be paired to serve as pitch wires.

Further, the power transmission part 300 of the surgical instrument for electrocautery 10 according to an embodiment of the present disclosure may include a coupling member 321, a coupling member 322, a coupling member 323, a coupling member 324, a coupling member 326, a coupling member 327 that are coupled to respective end portions of the wires to respectively couple the wires to the pulleys. Here, each of the coupling members may have various shapes as necessary, such as a ball shape, a tube shape, and the like.

Here, in the end tool 100 side, the coupling member 321 and the coupling member 322 may serve as a pitch wire-end tool coupling member, the coupling member 323 may serve as a first jaw wire-end tool coupling member, the coupling member 326 may serve as a second jaw wire-end tool coupling member.

Further, in the manipulation part 200 side, the coupling member 324 may serve as a first jaw wire-manipulation part coupling member, and the coupling member 327 may serve as a second jaw wire-manipulation part coupling member. In addition, although not shown in the drawings, a pitch wire-manipulation part coupling member may be further formed in the manipulation part 200 side.

The coupling relationship between the wires, the coupling members, and the respectively pulleys will be described in detail as follows.

First, the wires 301 and 305, which are first jaw wires, may be a single wire. The coupling member 323, which is a first jaw wire-end tool coupling member, is inserted at an intermediate point of the first jaw wire, which is a single wire, and the coupling member 323 is crimped and fixed, and then, both strands of the first jaw wire centered on the coupling member 323 may be referred to as the wire 301 and the wire 305, respectively.

Alternatively, the wires 301 and 305, which are first jaw wires, may also be formed as separate wires, and connected by the coupling member 323.

In addition, by coupling the coupling member 323 to a pulley 111, the wires 301 and 305 may be fixedly coupled to the pulley 111. This allows the pulley 111 to rotate as the wires 301 and 305 are pulled and released.

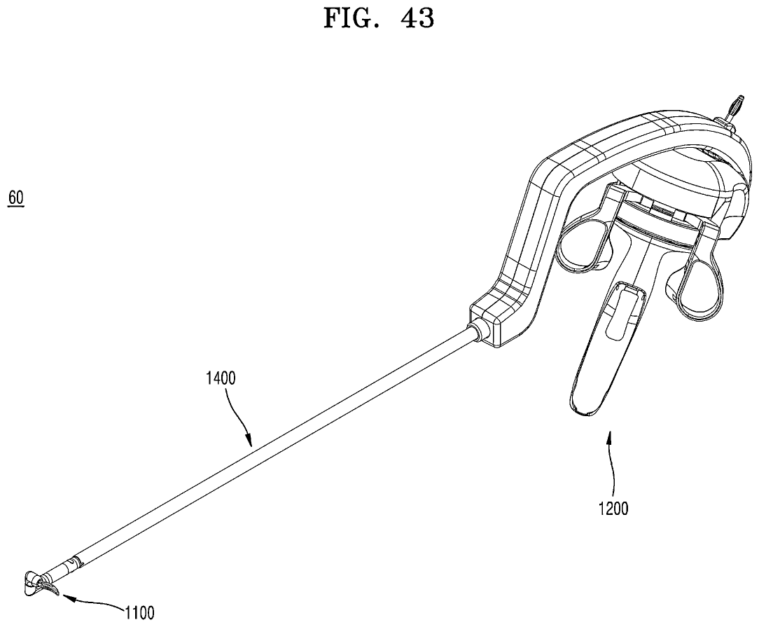

Meanwhile, the first jaw wire-manipulation part coupling member (see 324 of FIG. 43) may be coupled to end portions of the wires 301 and 305, which are opposite to one end portions to which the coupling member 323 is coupled.

In addition, by coupling the first jaw wire-manipulation part coupling member (see 324 of FIG. 43) to a pulley 210 as described above, the wires 301 and 305 may be fixedly coupled to the pulley 210 As a result, when the pulley 210 is rotated by a motor or a human force, the pulley 111 of the end tool 100 may be rotated as the wire 301 and the wire 305 are pulled and released.

In the same manner, each of the wires 302 and 306, which are second jaw wires, is coupled to the coupling member (see 326 of FIG. 43), which is a second jaw wire-end tool coupling member, and the second jaw wire-manipulation part coupling member (see 327 of FIG. 43). In addition, the coupling member (see 326 of FIG. 43) is coupled to a pulley 121, and the second jaw wire-manipulation part coupling member (see 327 of FIG. 43) is coupled to a pulley 220. As a result, when the pulley 220 is rotated by a motor or a human force, the pulley 121 of the end tool 100 may be rotated as the wire 302 and the wire 306 are pulled and released.

In the same manner, the wires 303 and 304, which are pitch wires, are respectively coupled to the coupling member (see 321 of FIG. 7), which is a pitch wire-end tool coupling member, and the pitch wire-manipulation part coupling member (not shown). In addition, the coupling member (see 321 of FIG. 7) is coupled to a pulley 131, and the pitch wire-manipulation part coupling member (not shown) is coupled to a pulley 231. As a result, when the pulley 231 is rotated by a motor or a human force, the pulley 131 of the end tool 100 may be rotated as the wire 303 and the wire 304 are pulled and released.

Here, in the surgical instrument for electrocautery 10 according to an embodiment of the present disclosure, a middle portion of each wire, that is, the inserted portion in the straight part 401 of the connection part 400, is replaced with a shrink tube and a shaft tube. Details thereof are described below with reference to FIGS. 14 to 16.

### (End Tool)

Hereinafter, the end tool 100 of the surgical instrument for electrocautery 10 of FIG. 2 will be described in more detail.

FIGS. 4 and 5 are perspective views illustrating the end tool of the surgical instrument for electrocautery of FIG. 2, FIG. 6 is an enlarged perspective view illustrating the end tool hub of the surgical instrument for electrocautery of FIG. 4, and FIGS. 9 and 10 are plan views illustrating the end tool of the surgical instrument for electrocautery of FIG. 2.

Here, FIG. 4 illustrates a state in which the end tool hub 180 and the pitch hub 107 are coupled, and FIG. 5 illustrates a state in which the end tool hub 180 and the pitch hub 107 are removed. Also, FIG. 9 is a view mainly showing wires, and FIG. 10 is a view mainly sowing pulleys.

Referring to FIGS. 4 through 10, the end tool 100 according to a first embodiment of the present disclosure includes a pair of jaws, that is, a first jaw 101 and a second jaw 102 for performing a grip motion. Here, each of the first jaw 101 and the second jaw 102, or a component encompassing the first jaw 101 and the second jaw 102 may be referred to as a jaw 103.

In addition, the end tool 100 may include a pulley 111, a pulley 113, a pulley 114, a pulley 115, and a pulley 116 related to a rotational motion of the first jaw 101. Furthermore, the end tool 100 may include a pulley 121, a pulley 123, a pulley 124, a pulley 125, and a pulley 126 related to a rotational motion of the second jaw 102.

Here, pulleys facing each other are shown disposed in parallel to each other, but are not limited thereto, and the location and size of each pulley may vary to be appropriate for the configuration of the end tool.

Also, the end tool 100 according to the first embodiment of the present disclosure may include an end tool hub 180 and a pitch hub 107.

A rotation shaft 141, which will be described later, may be inserted through the end tool hub 180. The pulley 111 and the pulley 121 that are axially coupled to the rotation shaft 141, and at least portions of the first jaw 101 and the second jaw 102 that are coupled to the pulleys 111 and 121 may be accommodated in the end tool hub 180. Here, in one embodiment of the present disclosure, the end tool hub 180 is provided with a guide part 183 that functions as an auxiliary pulley. That is, the guide part 183 that guides the paths of the wire 305 and the wire 302 may be disposed in the end tool hub 180. The guide part 183 of the end tool hub 180 may change the paths of the wires by acting as a kind of auxiliary pulley. The guide part 183 of the end tool hub 180 that plays the role of the auxiliary pulley will be explained in more detail later.

Meanwhile, the pulley 131 that functions as an end tool pitch pulley may be disposed on one end portion of the end tool hub 180. As illustrated in FIG. 4, the pulley 131 may be formed integrally with the end tool hub 180. Alternatively, the pulley 131 may be formed as a separate member from the end tool hub 180 and may be coupled to the end tool hub 180. The wire 303 and the wire 304 are coupled to the pulley 131, which acts as an end tool pitch pulley. The pulley 131 performs a pitch motion while rotating around the rotation shaft 143.

The rotation shaft 143 and a rotation shaft 144 may be inserted through the pitch hub 107, and the pitch hub 107 may be axially coupled to the end tool hub 180 and the pulley 131 by the rotation shaft 143. Accordingly, the end tool hub 180 and the pulley 131 may be formed to be pitch-rotated around the rotation shaft 143 with respect to the pitch hub 107.

In addition, the pitch hub 107 may accommodate therein at least portions of the pulley 113, pulley 114, pulley 123, and pulley 124 axially coupled to the rotation shaft 143. Also, the pitch hub 107 may accommodate therein at least portions of the pulley 115, pulley 116, pulley 125, and pulley 126 axially coupled to the rotation shaft 144.

In addition, the end tool 100 of the first embodiment of the present disclosure may include a rotation shaft 141, a rotation shaft 143, and a rotation shaft 144. As described above, the rotation shaft 141 may be inserted through the end tool hub 180, and the rotation shaft 143 and the rotation shaft 144 may be inserted through the pitch hub 107.

The rotation shaft 141, the rotation shaft 143, and the rotation shaft 144 may be disposed sequentially in a direction from a distal end 104 to a proximal end 105 of the end tool 100. Accordingly, starting from the distal end 104, the rotation shaft 141 may be referred to as a first pin, the rotation shaft 143 may be referred to as a third pin, and the rotation shaft 144 may be referred to as a fourth pin.

Here, the rotation shaft 141 may function as an end tool jaw pulley rotation shaft, the rotation shaft 143 may function as an end tool pitch rotation shaft, and the rotation shaft 144 may function as an end tool pitch auxiliary rotation shaft of the end tool 100.

One or more pulleys may be fitted to each of these rotation shafts 141, 143, and 144, which will be described in detail below.

The pulley 111 functions as an end tool first jaw pulley, and the pulley 121 functions as an end tool second jaw pulley. The pulley 111 may also be referred to as a first jaw pulley, and the pulley 121 may also be referred to as a second jaw pulley. These two components may be collectively referred to as an end tool jaw pulley.

The pulley 111 and the pulley 121, which are end tool jaw pulleys, are disposed to face each other and to be rotatable independently of each other around the rotation shaft 141, which is an end tool jaw pulley rotation shaft. Here, in the drawings, the pulley 111 and the pulley 121 rotate around one rotation shaft 141, but of course, each jaw pulley may be rotatable around a separate shaft. Here, the first jaw 101 may be fixedly coupled to the pulley 111 to rotate together with the pulley 111, and the second jaw 102 may be fixedly coupled to the pulley 121 to rotate together with the pulley 121. A yaw motion and an actuation motion of the end tool 100 are carried out, in response to the rotation of the pulley 111 and the pulley 121. That is, a yaw motion is performed when the pulley 111 and the pulley 121 rotate in the same direction around the rotation shaft 141, and an actuation motion is performed when the pulley 111 and the pulley 121 rotate in opposite directions around the rotation shaft 141.

Here, the first jaw 101 and the pulley 111 may be formed as separate members and coupled to each other, or the first jaw 101 and the pulley 111 may be formed as a one-body. Likewise, the second jaw 102 and the pulley 121 may be formed as separate members and coupled to each other, or the second jaw 102 and the pulley 121 may be formed as a one-body.

The guide part 183 of the end tool hub 180 may be formed on one side of the pulley 111 and the pulley 121, which are end tool jaw pulleys, and may serve as end tool jaw auxiliary pulleys. Specifically, the guide part 183 of the end tool hub 180 may be located on one side of the pulley 111 and the pulley 121. The guide part 183 of the end tool hub 180 will be explained in more detail later.

The pulley 113 and the pulley 114 may function as end tool first jaw pitch main pulleys, and the pulley 123 and the pulley 124 may function as end tool second jaw pitch main pulleys. These two components may also be collectively referred to as an end tool jaw pitch main pulley.

The pulley 115 and the pulley 116 may function as end tool first jaw pitch sub pulleys, and the pulley 125 and the pulley 126 may function as end tool second jaw pitch sub pulleys. These two components may also be collectively referred to as an end tool jaw pitch sub pulley.

Hereinafter, components related to the rotation of the pulley 111 will be described.

The pulley 113 and the pulley 114 function as end tool first jaw pitch main pulleys. In other words, the pulley 113 and the pulley 114 function as main rotation pulleys for the pitch motion of the first jaw 101. Here, the wire 301, which is a first jaw wire, is wound around the pulley 113, and the wire 305, which is a first jaw wire, is wound around the pulley 114.

The pulley 115 and the pulley 116 function as end tool first jaw pitch sub pulleys. In other words, the pulley 115 and the pulley 116 function as sub rotation pulleys for the pitch motion of the first jaw 101. Here, the wire 301, which is a first jaw wire, is wound around the pulley 115, and the wire 305, which is a first jaw wire, is wound around the pulley 116.

Here, the pulley 113 and the pulley 114 are disposed to face each other on one side of the pulley 111. Here, the pulley 113 and the pulley 114 are disposed to be rotatable independently of each other around the rotation shaft 143 that is an end tool pitch rotation shaft. Here, the pulley 115 and the pulley 116 are disposed to face each other on one side of the pulley 113 and one side of the pulley 114, respectively. Here, the pulley 115 and the pulley 116 are disposed to be rotatable independently of each other around the rotation shaft 144 that is an end tool pitch auxiliary rotation shaft. Here, in the drawings, it is illustrated that the pulley 113, the pulley 115, the pulley 114, and the pulley 116 are all formed to be rotatable around a Y-axis direction, but the concept of the present disclosure is not limited thereto, and the rotation shafts of the respective pulleys may be formed in various directions according to configurations thereof.

The wire 301, which is a first jaw wire, is sequentially wound to make contact with at least portions of the pulley 115, the pulley 113, and the pulley 111. In addition, the wire 305 connected to the wire 301 by the coupling member 323 is sequentially wound to make contact with at least portions of the pulley 111, the guide part 183, the pulley 114, and the pulley 116.

From another perspective, the wires 301 and 305, which are first jaw wires, are sequentially wound to make contact with at least portions of the pulley 115, the pulley 113, the pulley 111, the guide part 183, the pulley 114, and the pulley 116. The wire 301 and the wire 305 are formed to be movable along the above pulleys while rotating the pulleys.

Accordingly, when the wire 301 is pulled toward an arrow 301 of FIG. 9, the coupling member 323, to which the wire 301 is coupled, and the pulley 111 coupled to the coupling member 323 are rotated in a direction of an arrow L of FIG. 9. On the other hand, when the wire 305 is pulled toward an arrow 305 of FIG. 9, the coupling member 323, to which the wire 305 is coupled, and the pulley 111 coupled to the coupling member 323 are rotated in a direction of an arrow R of FIG. 9.

Hereinafter, components related to the rotation of the pulley 121 will be described.

The pulley 123 and the pulley 124 function as end tool second jaw pitch main pulleys. In other words, the pulley 123 and the pulley 124 function as main rotation pulleys for the pitch motion of the second jaw 102. Here, the wire 306, which is a second jaw wire, is wound around the pulley 123, and the wire 302, which is a second jaw wire, is wound around the pulley 124.

The pulley 125 and the pulley 126 function as end tool second jaw sub pulleys. In other words, the pulley 125 and the pulley 126 function as sub rotation pulleys for the pitch motion of the second jaw 102. Here, the wire 306, which is a second jaw wire, is wound around the pulley 125, and the wire 302, which is a second jaw wire, is wound around the pulley 126.

The pulley 123 and the pulley 124 are disposed to face each other on one side of the pulley 121. Here, the pulley 123 and the pulley 124 are disposed to be rotatable independently of each other around the rotation shaft 143 that is an end tool pitch rotation shaft. Here, the pulley 125 and the pulley 126 are disposed to face each other on one side of the pulley 123 and one side of the pulley 124, respectively. Here, the pulley 125 and the pulley 126 are disposed to be rotatable independently of each other around the rotation shaft 144 that is an end tool pitch auxiliary rotation shaft. Here, in the drawings, it is illustrated that the pulley 123, the pulley 125, the pulley 124, and the pulley 126 are all formed to be rotatable around the Y-axis direction, but the concept of the present disclosure is not limited thereto, and the rotation shafts of the respective pulleys may be formed in various directions according to configurations thereof.

The wire 306, which is a second jaw wire, is sequentially wound to make contact with at least portions of the pulley 125, the pulley 123, and the pulley 121. In addition, the wire 302 connected to the wire 306 by the coupling member 326 is sequentially wound to make contact with at least portions of the pulley 121, the guide part 183, the pulley 124, and the pulley 126.

From another perspective, the wires 306 and 302, which are second jaw wires, are sequentially wound to make contact with at least portions of the pulley 125, the pulley 123, the pulley 121, the guide part 183, the pulley 124, and the pulley 126. The wire 306 and the wire 302 are formed to be movable along the above pulleys while rotating the pulleys.

Accordingly, when the wire 306 is pulled toward an arrow 306 of FIG. 9, the coupling member 322, to which the wire 306 is coupled, and the pulley 121 coupled to the coupling member 323 are rotated in the direction of the arrow R of FIG. 9. On the other hand, when the wire 302 is pulled toward an arrow 302 of FIG. 9, the coupling member 326, to which the wire 302 is coupled, and the pulley 121 coupled to the coupling member 323 are rotated in the direction of the arrow L of FIG. 9.

Hereinafter, the end tool hub 180 according to one embodiment of the present disclosure will be described in more detail, and in particular, a description will be given, focusing on the guide part 183 of the end tool hub 180, which serves as an auxiliary pulley.

The end tool hub 180 includes a pair of jaw pulley coupling parts 181 and 182, a guide part 183, a guide groove 184, and a pitch pulley part 185.

Specifically, the pair of jaw pulley coupling parts 181 and 182 are formed to face each other to accommodate the pulley 111 and the pulley 121 therein. In addition, a through hole is formed through each of the jaw pulley coupling parts 181 and 182, and the rotation shaft 141 is inserted through the jaw pulley coupling parts 181 and 182 and the pulleys 111 and 121 to axially couple the pulley coupling parts 181 and 182 and the pulleys 111 and 121.

The pair of jaw pulley coupling parts 181 and 182 are connected by the guide part 183. That is, the pair of jaw pulley coupling parts 181 and 182 that are parallel to each other may be coupled to each other by the guide part 183, which is formed in a direction approximately perpendicular to the pair of jaw pulley coupling parts 181 and 182. Accordingly, the pair of jaw pulley coupling parts 181 and 182 may form a shape like “⊏” together with the guide part 183, such that the pulley 111 and the pulley 121 are accommodated.

To explain this from another perspective, it may be understood that the pair of jaw pulley coupling parts 181 and 182 extend in an X-axis direction from both end portions of the guide part 183 that is formed long in a Z-axis direction.

Here, the guide part 183 may be formed in a cylindrical shape with an approximately semicircular cross-section. This semicircular portion may be disposed to protrude toward the pulley 111 and the pulley 121. Expressing this from another perspective, it may be expressed that the guide part 183 protrudes toward a space defined by the pair of jaw pulley coupling parts 181 and 182 and the guide part 183. Expressing this from another perspective, it may be expressed that an area of the guide part 183, which is adjacent to the jaw pulley coupling parts 181 and 182, has a cross-section curved at a predetermined curvature.

Or, to express this from another perspective, it may also be said that the guide part 183 has the wire 305 and the wire 302 wound around its outer peripheral surface and thus functions as a kind of pulley member for guiding the paths of the wire 305 and the wire 302. However, here, it may be understood that the guide part 183 is not a member that rotates around a predetermined shaft like a pulley in the original sense but is fixed as a portion of the end tool hub 180, with a wire wound therearound, to similarly perform some of functions of a pulley for guiding the path of the wire.

Here, in the drawings, the guide part 183 is shown formed in a cylindrical shape with an approximately semicircular cross-section. That is, at least a portion of the cross-section of the guide part 183 on an XY plane is shown forming a predetermined arcuate shape. However, the concept of the present disclosure is not limited thereto, and the guide part 183 may be formed to have a cross-section with a predetermined curvature, such as an oval or parabola, or may be formed in the form of a polygonal pillar with edges rounded to a certain extent, to have various shapes and sizes suitable for guiding the paths of the wires 305 and 302.

Here, a guide groove 184 may be further formed in a portion of the guide part 183 that is in contact with the wire 305 and the wire 302, to better guide the paths of the wire 305 and the wire 302. The guide groove 184 may be formed in the form of a groove that is recessed to a certain extent from a protruded surface of the guide part 183.

Here, the drawings show that the guide groove 184 is formed in the entire arcuate surface of the guide part 183, but the concept of the present disclosure is not limited thereto. The guide groove 184 may alternatively be formed only in a portion of the arcuate surface of the guide part 183, if necessary.

By further forming the guide groove 184 in the guide part 183 in this way, unnecessary friction with the wires may be reduced, thereby improving the durability of the wires.

A pitch pulley part 185 may be further formed on the guide part 183 in a direction opposite to a direction that the jaw pulley coupling parts 181 and 182 are formed. The pitch pulley part 185 may be provided with a pulley 131 as a pitch pulley, around which the wires 303 and 304 as pitch wires are wound. However, here, it may be understood that the pulley 131 is not a member that rotates around a predetermined shaft like a pulley in the original sense but is fixed as a portion of the end tool hub 180, with a wire wound therearound, to similarly perform some of functions of a pulley for guiding the path of the wire. Here, the pitch pulley part 185 may be formed on the XZ plane. In addition, a through hole through which the rotation shaft 143 can be inserted may be formed through the pitch pulley part 185.

Meanwhile, although not illustrated in the drawings, the pitch pulley part and the pitch pulley may be formed as separate members and coupled to each other, and the rotation shaft 143 may be formed to be inserted through the pitch pulley part and the pulley.

Hereinafter, the role and function of the guide part 183 will be described in more detail.

The guide part 183 may be in contact with the wire 305 and the wire 302 to change the disposition paths of the wire 305 and the wire 302 to a certain extent, thereby playing a role of expanding the radius of rotation of each of the first jaw 101 and the second jaw 102.

That is, when the auxiliary pulley or guide part 183 is not disposed, each of the pulley 111, which is the first jaw pulley, and the pulley 121, which is the second jaw pulley, is rotatable up to a right angle, but in one embodiment of the present disclosure, the maximum rotation angle of each pulley can increase by virtue of the guide part 183 additionally disposed in the end tool hub 180.

This enables a motion that two jaws of the end tool 100 must be apart from each other for an actuation motion in a state where the two jaws are yaw-rotated by 90°. In other words, with the configuration of the guide part 183 of the end tool hub 180, the range of yaw rotation enabling the actuation motion may be expanded.

Furthermore, without addition of a separate structure such as an auxiliary pulley, the guide part 183 may be formed in the existing end tool hub 180, resulting in expanding the rotation range without additional components and manufacturing processes.

In this way, without the additional structure for expanding the rotation angle, the number of components can be reduced and the manufacturing process can be simplified. Also, as the length of the end tool is shortened by the size of the auxiliary pulley, the length of the end tool upon performing the pitch motion can be shortened, which may more facilitate surgical operations to be performed in a narrow space.

This will be explained in more detail as follows.

The end tool 100 of the surgical instrument according to one embodiment of the present disclosure includes the guide part 183 that is disposed on an inner wall of the end tool hub 180 to change the path of the wire, thereby changing the disposition path of the wire without a separate structure. In this way, the guide part 183 is formed in the end tool hub 180 to change the disposition paths of the wire 305 and the wire 302 to a certain extent. Accordingly, a tangential direction of the wire 305 and the wire 302 may be changed, and thus the rotation angle of the coupling member 323 and the coupling member 326 for coupling the wires to the pulleys is increased.

That is, the coupling member 326 that couples the wire 302 to the pulley 121 is rotatable until it is located on a common internal tangent of the pulley 121 and the guide part 183. Likewise, the coupling member (see 323 in FIG. 13) that couples the wire 305 to the pulley 111 is rotatable until it is located on a common internal tangent of the pulley 111 and the guide part 183. This may increase the rotation angle of the coupling member (see 323 in FIG. 13).

To explain this from another perspective, the wire 301 and the wire 305 wound around the pulley 111 by the guide part 183 are disposed on one side based on a plane that is perpendicular to the Y axis and passes through the X axis. Simultaneously, the wire 302 and the wire 306 wound around the pulley 121 by the guide part 183 are disposed on another side based on a plane that is perpendicular to the Y axis and passes through the X axis.

In other words, the pulleys 113 and 114 are disposed on one side based on the plane that is perpendicular to the Y axis and passes through the X axis, and the pulleys 123 and 124 are disposed on another side based on the plane that is perpendicular to the Y axis and passes through the X axis.

In other words, the wire 305 is located on the internal tangent of the pulley 111 and the guide part 183, and the rotation angle of the pulley 111 is increased by the guide part 183. Furthermore, the wire 302 is located on the internal tangent of the pulley 121 and the guide part 183, and the rotation angle of the pulley 121 is increased by the guide part 183.

Compared to a surgical instrument with a separate auxiliary pulley, the length of the end tool may be shortened in the surgical instrument according to the embodiment of the present disclosure, in which an auxiliary pulley is not disposed and the guide part 183 is disposed on the inner wall of the end tool hub 180 to change the path of the wire. As the length of the end tool is shorted, the operator's manipulation may become easier when performing a surgery in a narrow surgical space within the human body, and the side effects of the surgery may be reduced.

In this way, according to the present disclosure, the radius of rotation of the pulley 111 as the first jaw pulley and the pulley 121 as the second jaw pulley can be widened, thereby obtaining an effect of increasing a yaw-motion range in which a normal opening and closing actuation motion is performed.

Meanwhile, as described above, although the guide portion 183 may be formed integrally within the end tool hub 180, the guide portion 183 may also be formed as a separate member from the end tool hub 180. That is, as shown in FIG. 7, the guide member 150 may be formed separately and combined into the end tool hub 180.

Here, the guide member 150 may include an elastic material, such as plastic, and guide paths of the wire 305 and the wire 302 to reduce friction between the wire and the end tool hub 180. In other words, the guide member 150 formed of plastic may be located between the end tool hub 180 and the wire so that the wire may not be in direct contact with the end tool hub 180 formed of a metal material, thereby obtaining the effect of reducing friction and wear of the wire.

In addition, the guide member 150 may have a through-hole 155 formed therein, so that when the guide member 150 is located within the end tool hub 180, a second pin (not shown) may be sequentially inserted into the end tool hub 180 and the through-hole 155 of the guide member 150, thereby fixedly coupling the end tool hub 180 to the guide member 150.

In addition, like the shape of the guide portion 183 described above, at least a portion of a cross-section of the guide member 150 may have an arc shape in the XY plane, but the concept of the present disclosure is not limited thereto and the cross-section of the guide member 150 may have various shapes and sizes suitable to guide the paths of the wire 305 and the wire 302, for example, have a curvature, such as an oval or parabola, or have a polygonal column having the corners rounded to a certain degree.

Referring to FIGS. 7 and 8, guide wings 153 and 154 may be further formed in portions of the guide member 150 that contact the wire 305 and wire 302 to better guide the paths of the wires. The guide wings 153 and 154 may be formed in the form of wings that protrude to a certain degree from a surface of the guide member 150.

In detail, the guide wings 153 and 154 may be formed to have a path corresponding to a moving direction in which each of the wire 305 and the wire 302 moves from a point at which the wire 305 and the wire 302 meet the guide member 150 toward the end tool pitch pulleys 114 and 124 through the end tool hub 180.

From another perspective, the wire 305 and the wire 302 may be located to be in contact with inner edges 153a and 154a between the guide member 150 and the guide wings 153 and 154 protruding from a surface of the guide member, and the inner edges 153a and 154a may be formed as curved surfaces.

Meanwhile, as shown in the drawing, the guide wing 154 may be formed on one side of the guide member 150 in which the wire 305 is in contact with the guide member 150, and the guide wing 153 may be formed on the other side in which the wire 302 is in contact with the guide member 150.

Here, the drawing shows that the guide wings 153 and 154 are formed only on the side of the guide member 150, but the concept of the present disclosure is not limited thereto, and the guide wing 153 may be formed on the entire arc surface of the guide member 150.

Meanwhile, here, the pitch wire may be a single wire. That is, after inserting a fastening member 325 into a middle point of the pitch wire, which is a single wire, and crimping the fastening member 325 to be fixed to the pitch wire, both strands of the pitch wire based on the fastening member 325 may be referred to as the wire 303 and the wire 304, respectively. At this time, in order to prevent the fastening member 325 of the pitch wire from being separated from the end tool hub 180, the fastening member 325 to which the pitch wire is fastened may be located in a tube accommodating portion 186 of the end tool hub 180 and the guide member 150 may be coupled to the end tool hub 180 so that the guide member 150 may seal the tube accommodating portion 186.

In addition, the guide member 150 has a recess 152 having a certain depth from a surface coupled to the end tool hub 180 to accommodate the fastening member 325 partially protruding from the tube accommodating portion 186 toward the guide member 150.

By coupling the guide member 150, which is formed as a separate member, to the end tool hub 180, friction and wear due to contact between the wire 305 and the wire 302 and the end tool hub 180 may be reduced, and the fastening member 325 of the pitch wire configured as a single wire may be stably coupled to the end tool hub 180.

Hereinafter, the pitch motion according to the present disclosure will be described in more detail.

Meanwhile, when the wire 301 is pulled toward an arrow 301 of FIG. 9 and simultaneously the wire 305 is pulled toward an arrow 305 of FIG. 9 (i.e., when both strands of the first jaw wire are pulled), the pulley 111, to which the wire 301 and the wire 305 are fixedly coupled, and the end tool hub 180, to which the pulley 111 is coupled, are rotated together in a counterclockwise direction around the rotation shaft 143, because the wire 301 and the wire 305, as illustrated in FIG. 5, are wound around lower portions of the pulley 113 and the pulley 114 rotatable around the rotation shaft 143, which is an end tool pitch rotation shaft. Accordingly, the end tool 100 performs a pitch motion while rotating downward. In this case, since the second jaw 102 and the wires 302 and 306 fixedly coupled to the second jaw 102 are wound around upper portions of the pulleys 123 and 124 rotatable around the rotation shaft 143, the wire 302 and the wire 306 are unwound in the opposite directions of arrows 302 and 306, respectively.

In contrast, when the wire 302 is pulled toward an arrow 302 of FIG. 9 and simultaneously the wire 306 is pulled toward an arrow 306 of FIG. 9, the pulley 121, to which the wire 302 and the wire 306 are fixedly coupled, and the end tool hub 180, to which the pulley 121 is coupled, are rotated together in a clockwise direction around the rotation shaft 143, because the wire 302 and the wire 306, as illustrated in FIG. 5, are wound around upper portions of the pulley 123 and the pulley 124 rotatable around the rotation shaft 143, which is an end tool pitch rotation shaft. Accordingly, the end tool 100 performs a pitch motion while rotating upward. In this case, since the first jaw 101 and the wires 301 and 305 fixedly coupled to the first jaw 101 are wound around lower portions of the pulleys 113 and 114 rotatable around the rotation shaft 143, the wire 302 and the wire 306 are moved in the opposite directions of arrows 301 and 305, respectively.

Meanwhile, the end tool 100 of the surgical instrument for electrocautery 10 according to the present disclosure may further include the pulley 131, which is an end tool pitch pulley, and the manipulation part 200 may further include a pulley 231 and a pulley 232, which are manipulation part pitch pulleys. The power transmission part 300 may further include a wire 303 and a wire 304 that are pitch wires. In detail, the pulley 131 of the end tool 100 may be rotatable around the rotation shaft 143, which is an end tool pitch rotation shaft, and may be integrally formed with the end tool hub 180 (or fixedly coupled to the end tool hub 180). In addition, the wire 303 and the wire 304 may serve to connect the pulley 131 of the end tool 100 to the pulley 231 and the pulley 232 of the manipulation part 200.

Accordingly, when the pulley 231 and the pulley 232 of the manipulation part 200 rotate, the rotation of the pulley 231 and the pulley 232 is transmitted to the pulley 131 of the end tool 100 through the wire 303 and the wire 304, so that the pulley 131 is rotated together. Consequently, the end tool 100 performs a pitch motion while rotating.

That is, as the surgical instrument for electrocautery 10 according to the first embodiment of the present disclosure includes the pulley 131 of the end tool 100, the pulleys 231 and 232 of the manipulation part 200, and the wires 303 and 304 of the power transmission part 300, to transmit a driving force for a pitch motion, the driving force for the pitch motion of the manipulation part 200 may be more completely transmitted to the end tool 100, thereby improving motion reliability.

Here, the diameters of the pulley 113, the pulley 114, the pulley 123, and the pulley 124, which are end tool jaw pitch main pulleys, and the diameter of the pulley 131, which is an end tool pitch pulley, may be the same as or different from each other. In this case, a ratio of the diameter of the end tool pitch pulley to the diameter of the end tool jaw pitch main pulley may be the same as a ratio of the diameter of the manipulation part pitch main pulley to the diameter of the manipulation part pitch pulley of the manipulation part 200, which will be described later.

### (Manipulation Part)

FIGS. 11 and 12 are perspective views illustrating the manipulation part of the surgical instrument of FIG. 2.

Referring to FIGS. 2 to 12, the manipulation part 200 of the surgical instrument for electrocautery 10 according to the first embodiment of the present disclosure includes a first handle 204 that a user can grip, the actuation manipulation part 203 configured to control an actuation motion of the end tool 100, the yaw manipulation part 202 configured to control a yaw motion of the end tool 100, and the pitch manipulation part 201 configured to control a pitch motion of the end tool 100.

The manipulation part 200 may include the pulley 210, a pulley 211, a pulley 212, a pulley 213, a pulley 214, a pulley 215, a pulley 216, a pulley 217, and a pulley 218 that are related to a rotational motion of the first jaw 101. In addition, the manipulation part 200 may include the pulley 220, a pulley 221, a pulley 222, a pulley 223, a pulley 224, a pulley 225, a pulley 226, a pulley 227, and a pulley 228 that are related to a rotational motion of the second jaw 102. In addition, the manipulation part 200 may include the pulley 231, the pulley 232, a pulley 233, and a pulley 234 that are related to a pitch motion thereof. In addition, the manipulation part 200 may include a pulley 235, which is a relay pulley disposed at some places along the bent part 402 of the connection part 400.

Here, the pulleys facing each other are illustrated in the drawings as being formed parallel to each other, but the concept of the present disclosure is not limited thereto, and each of the pulleys may be variously formed with a position and a size suitable for the configuration of the manipulation part.

Further, the manipulation part 200 of the first embodiment of the present disclosure may include a rotation shaft 241, a rotation shaft 242, the rotation shaft 243, a rotation shaft 244, a rotation shaft 245, and the rotation shaft 246. Here, the rotation shaft 241 may function as a manipulation part first jaw actuation rotation shaft, and the rotation shaft 242 may function as a manipulation part second jaw actuation rotation shaft. In addition, the rotation shaft 243 may function as a manipulation part yaw main rotation shaft, and the rotation shaft 244 may function as a manipulation part yaw sub-rotation shaft. In addition, the rotation shaft 245 may function as a manipulation part pitch sub-rotation shaft, and the rotation shaft 246 may function as a manipulation part pitch main rotation shaft.

The rotation shaft 241/rotation shaft 242, the rotation shaft 243, the rotation shaft 244, the rotation shaft 245, and the rotation shaft 246 may be sequentially disposed from a distal end 205 of the manipulation part 200 toward a proximal end 206.

Each of the rotation shafts 241, 242, 243, 244, 245, and 246 may be fitted into one or more pulleys, which will be described in detail later.

The pulley 210 functions as a manipulation part first jaw actuation pulley, the pulley 220 functions as a manipulation part second jaw actuation pulley, and these components may also be collectively referred to as a manipulation part actuation pulley.

The pulley 211 and the pulley 212 function as manipulation part first jaw yaw main pulleys, the pulley 221 and the pulley 222 function as manipulation part second jaw yaw main pulleys, and these components may also be collectively referred to as a manipulation part yaw main pulley.

The pulley 213 and the pulley 214 function as manipulation part first jaw yaw sub-pulleys, the pulley 223 and the pulley 224 function as manipulation part second jaw yaw sub-pulleys, and these components may also be collectively referred to as a manipulation part yaw sub-pulley.

The pulley 215 and the pulley 216 function as manipulation part first jaw pitch sub-pulleys, the pulley 225 and the pulley 226 function as manipulation part second jaw pitch sub-pulleys, and these components may also be collectively referred to as a manipulation part pitch sub-pulley.

The pulley 217 and the pulley 218 function as manipulation part first jaw pitch main pulleys, and the pulley 227 and the pulley 228 function as manipulation part second jaw pitch main pulleys, and these components may also be collectively referred to as the manipulation part pitch main pulley.

The pulley 231 and the pulley 232 function as manipulation part pitch main pulleys, and the pulley 233 and the pulley 234 function as manipulation part pitch sub-pulleys.

The above components are categorized from the perspective of the manipulation part for each motion (pitch/yaw/actuation) as follows.

The pitch manipulation part 201 configured to control a pitch motion of the end tool 100 may include the pulley 215, the pulley 216, the pulley 217, the pulley 218, the pulley 225, the pulley 226, the pulley 227, the pulley 228, the pulley 231, the pulley 232, and the pulley 234. In addition, the pitch manipulation part 201 may include the rotation shaft 245 and the rotation shaft 246. In addition, the pitch manipulation part 201 may further include a pitch frame 208.

The yaw manipulation part 202 configured to control a yaw motion of the end tool 100 may include the pulley 211, the pulley 212, the pulley 213, the pulley 214, the pulley 221, the pulley 222, the pulley 223, and the pulley 224. In addition, the yaw manipulation part 202 may include the rotation shaft 243 and the rotation shaft 244. In addition, the yaw manipulation part 202 may further include a yaw frame 207.

The actuation manipulation part 203 configured to control an actuation motion of the end tool 100 may include the pulley 210, the pulley 220, the rotation shaft 241, and the rotation shaft 242. In addition, the actuation manipulation part 203 may further include the first actuation manipulation part 251 and the second actuation manipulation part 256.

Hereinafter, each component of the manipulation part 200 will be described in more detail.

The first handle 204 may be formed to be gripped by a user with the hand, and in particular, may be formed to be grasped by the user by wrapping the first handle 204 with his/her palm. In addition, the actuation manipulation part 203 and the yaw manipulation part 202 are formed on the first handle 204, and the pitch manipulation part 201 is formed on one side of the yaw manipulation part 202. In addition, the other end portion of the pitch manipulation part 201 is connected to the bent part 402 of the connection part 400.

The actuation manipulation part 203 includes the first actuation manipulation part 251 and the second actuation manipulation part 256. The first actuation manipulation part 251 includes the rotation shaft 241, the pulley 210, the first actuation extension part 252, and a first actuation gear 253. The second actuation manipulation part 256 includes the rotation shaft 242, the pulley 220, the second actuation extension part 257, and a second actuation gear 258. Here, end portions of the first actuation extension part 252 and the second actuation extension part 257 are formed in the shape of a hand ring, which may act as a second handle.

Here, the rotation shaft 241 and the rotation shaft 242, which are actuation rotation axes, may be formed to form a predetermined angle with an XY plane on which the connection part 400 is formed. For example, the rotation shaft 241 and the rotation shaft 242 may be formed in a direction parallel to the Z-axis, and in this state, when the pitch manipulation part 201 or the yaw manipulation part 202 is rotated, the coordinate system of the actuation manipulation part 203 may change relatively. Of course, the concept of the present disclosure is not limited thereto, and the rotation shaft 241 and the rotation shaft 242 may be formed in various directions so as to be suitable for a structure of the hand of the user gripping the actuation manipulation part 203 according to an ergonomic design.

Meanwhile, the pulley 210, the first actuation extension part 252, and the first actuation gear 253 are fixedly coupled to each other to be rotatable together around the rotation shaft 241. Here, the pulley 210 may be configured to be a single pulley or two pulleys fixedly coupled to each other.

Similarly, the pulley 220, the second actuation extension part 257, and the second actuation gear 258 are fixedly coupled to each other to be rotatable together around the rotation shaft 242. Here, the pulley 220 may be configured to be a single pulley or two pulleys fixedly coupled to each other.

Here, the first actuation gear 253 and the second actuation gear 258 are formed to be engaged with each other such that, when any one gear is rotated in one direction, the other gear is rotated together in a direction opposite to the one direction.

The yaw manipulation part 202 may include the rotation shaft 243, the pulleys 211 and 212, which are manipulation part first jaw yaw main pulleys, the pulleys 221 and 222, which are manipulation part second jaw yaw main pulleys, and the yaw frame 207. In addition, the yaw manipulation part 202 may further include the pulleys 213 and 214, which are manipulation part first jaw yaw sub-pulleys formed on one side of the pulleys 211 and 212, and the pulleys 223 and 224 that are manipulation part second jaw yaw sub-pulleys formed on one side of the pulleys 221 and 222. Here, the pulleys 213 and 214 and the pulleys 223 and 224 may be coupled to the pitch frame 208 to be described later.

Here, it is illustrated in the drawings that the yaw manipulation part 202 includes the pulleys 211 and 212 and the pulleys 221 and 222, wherein the pulleys 211 and 212 and the pulleys 221 and 222 are each provided with two pulleys formed to face each other and independently rotatable, but the concept of the present disclosure is not limited thereto. That is, one or more pulleys having the same diameter or different diameters may be provided according to the configuration of the yaw manipulation part 202.

In detail, the rotation shaft 243, which is a manipulation part yaw main rotation shaft, is formed on one side of the actuation manipulation part 203 on the first handle 204. At this time, the first handle 204 is formed to be rotatable around the rotation shaft 243.

Here, the rotation shaft 243 may be formed to form a predetermined angle with the XY plane on which the connection part 400 is formed. For example, the rotation shaft 243 may be formed in a direction parallel to the Z-axis, and in this state, when the pitch manipulation part 201 is rotated, the coordinate system of the rotation shaft 243 may change relatively as described above. Of course, the concept of the present disclosure is not limited thereto, and the rotation shaft 243 may be formed in various directions so as to be suitable for a structure of the hand of the user gripping the manipulation part 200 according to an ergonomic design.

Meanwhile, the pulleys 211 and 212 and the pulleys 221 and 222 are coupled to the rotation shaft 243 so as to be rotatable around the rotation shaft 243. In addition, the wire 301 or the wire 305, which is a first jaw wire, is wound around the pulleys 211 and 212, and the wire 302 or the wire 306, which is a second jaw wire, may be wound around the pulleys 221 and 222. In this case, the pulleys 211 and 212 and the pulleys 221 and 222 may each be configured as two pulleys formed to face each other and independently rotatable. Accordingly, a wire being wound and a wire being released may be wound around respective separate pulleys so that the wires may perform motions without interference with each other.

The yaw frame 207 rigidly connects the first handle 204, the rotation shaft 241, the rotation shaft 242, and the rotation shaft 243, so that the first handle 204, the yaw manipulation part 202, and the actuation manipulation part 203 are integrally yaw-rotated around the rotation shaft 243.

The pitch manipulation part 201 may include the rotation shaft 246, the pulley 217 and the pulley 218, which are manipulation part first jaw pitch main pulleys, the pulleys 227 and 228, which are manipulation part second jaw pitch main pulleys, and the pitch frame 208. In addition, the pitch manipulation part 201 may further include the rotation shaft 245, the pulleys 215 and 216, which are manipulation part first jaw pitch sub-pulleys formed on one side of the pulley 217 and the pulley 218, and the pulleys 225 and 226, which are manipulation part second jaw pitch sub-pulleys formed on one side of the pulley 227 and the pulley 228. The pitch manipulation part 201 may be connected to the bent part 402 of the connection part 400 through the rotation shaft 246.

In detail, the pitch frame 208 is a base frame of the pitch manipulation part 201, and the rotation shaft 243 is rotatably coupled to one end portion thereof. That is, the yaw frame 207 is formed to be rotatable around the rotation shaft 243 with respect to the pitch frame 208.

As described above, since the yaw frame 207 connects the first handle 204, the rotation shaft 243, the rotation shaft 241, and the rotation shaft 242, and the yaw frame 207 is also axially coupled to the pitch frame 208, when the pitch frame 208 is pitch-rotated around the rotation shaft 246, the yaw frame 207 connected to the pitch frame 208, the first handle 204, the rotation shaft 241, the rotation shaft 242, and the rotation shaft 243 are pitch-rotated together. That is, when the pitch manipulation part 201 is rotated around the rotation shaft 246, the actuation manipulation part 203 and the yaw manipulation part 202 are rotated together with the pitch manipulation part 201. In other words, when a user pitch-rotates the first handle 204 around the rotation shaft 246, the actuation manipulation part 203, the yaw manipulation part 202, and the pitch manipulation part 201 are moved together.

The pulleys 217 and 218 and the pulleys 227 and 228 are coupled to the rotation shaft 246 so as to be rotatable around the rotation shaft 246 of the pitch frame 208.

Here, the pulley 217 and the pulley 218 may be formed to face each other so as to be independently rotatable. Accordingly, a wire being wound and a wire being released may be wound around respective separate pulleys so that the wires may perform motions without interference with each other. Similarly, the pulley 227 and the pulley 228 may also be formed to face each other so as to be independently rotatable. Accordingly, a wire being wound and a wire being released may be wound around respective separate pulleys so that the wires may perform motions without interference with each other.

Next, a motion of each of the wires 303 and 304, which are pitch wires, is described as follows.

The pulley 131, which is an end tool pitch pulley, is fixedly coupled to the end tool hub 180 in the end tool 100, and the pulley 231 and the pulley 232, which are manipulation part pitch pulleys, are fixedly coupled to the pitch frame 208 in the manipulation part 200. In addition, these pulleys are connected to each other by the wires 303 and 304, which are pitch wires, so that a pitch motion of the end tool 100 may be performed more easily according to the pitch manipulation of the manipulation part 200. Here, the wire 303 is fixedly coupled to the pitch frame 208 via the pulley 231 and the pulley 233, and the wire 304 is fixedly coupled to the pitch frame 208 via the pulley 232 and the pulley 234. That is, the pitch frame 208 and the pulleys 231 and 232 are rotated together around the rotation shaft 246 by the pitch rotation of the manipulation part 200, and as a result, the wires 303 and 304 are also moved, and thus, a driving force of additional pitch rotation may be transmitted separately from the pitch motion of the end tool by the wire 301, the wire 302, the wire 305, and the wire 306, which are jaw wires.

A connection relationship of each of the first handle 204, the pitch manipulation part 201, the yaw manipulation part 202, and the actuation manipulation part 203 is summarized as follows. The rotation shafts 241 and 242, the rotation shaft 243, the rotation shaft 244, the rotation shaft 245, and the rotation shaft 246 may be formed on the first handle 204. In this case, since the rotation shafts 241 and 242 are directly formed on the first handle 204, the first handle 204 and the actuation manipulation part 203 may be directly connected to each other. Meanwhile, since the rotation shaft 243 is directly formed on the first handle 204, the first handle 204 and the yaw manipulation part 202 may be directly connected to each other. On the other hand, since the pitch manipulation part 201 is formed on one side of the yaw manipulation part 202 so as to be connected to the yaw manipulation part 202, the pitch manipulation part 201 is not directly connected to the first handle 204, and the pitch manipulation part 201 and the first handle 204 may be formed to be indirectly connected to each other via the yaw manipulation part 202.

Continuing to refer to the drawings, in the surgical instrument for electrocautery 10 according to the first embodiment of the present disclosure, the pitch manipulation part 201 and the end tool 100 may be formed on the same or parallel axis (X-axis). That is, the rotation shaft 246 of the pitch manipulation part 201 is formed at one end portion of the bent part 402 of the connection part 400, and the end tool 100 is formed at the other end portion of the connection part 400.

In addition, one or more relay pulleys 235 configured to change or guide paths of the wires may be disposed at some places along the connection part 400, particularly in the bent part 402. As at least some of the wires are wound around the relay pulleys 235 to guide the paths of the wires, these wires may be disposed along a bent shape of the bent part 402.

Here, in the drawings, it is illustrated that the connection part 400 is formed to be curved with a predetermined curvature by having the bent part 402, but the concept of the present disclosure is not limited thereto, and the connection part 400 may be formed linearly or to be bent one or more times as necessary, and even in this case, it may be said that the pitch manipulation part 201 and the end tool 100 are formed on substantially the same axis or parallel axes. In addition, although FIG. 3 illustrates that each of the pitch manipulation part 201 and the end tool 100 is formed on an axis parallel to the X-axis, the concept of the present disclosure is not limited thereto, and the pitch manipulation part 201 and the end tool 100 may be formed on different axes.

The staple manipulation part 260 is connected to the staple pulley 161 of the end tool 100 by the wires 307 and 308, which are staple wires, and serves to alternately rotate the staple pulley 161 in the clockwise or counterclockwise direction.

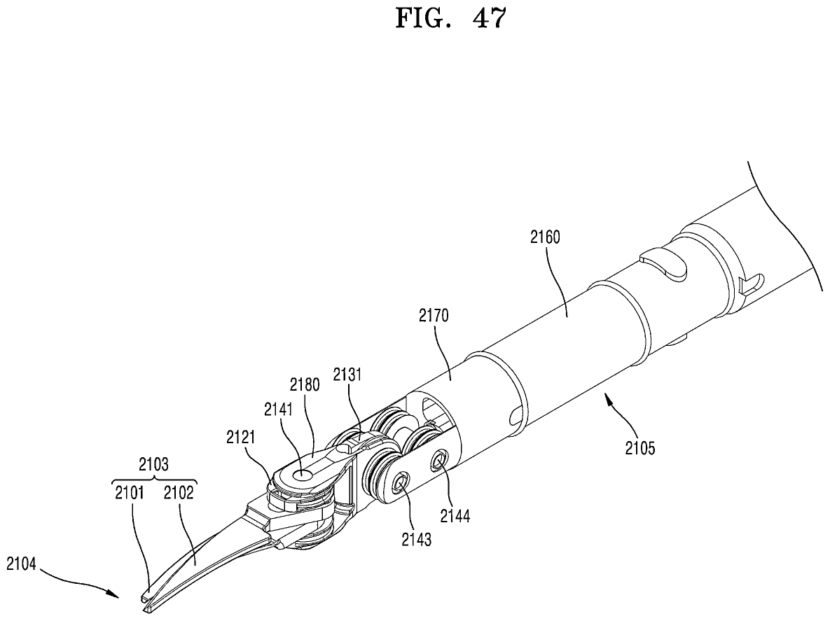

To this end, although not shown in the drawings, the staple manipulation part 260 may include a motor (not shown). That is, the motor (not shown) is driven while the user presses the staple manipulation part 260 formed in the form of a button to alternately rotate the manipulation part staple pulley (see 269 of FIG. 47) in the clockwise or counterclockwise direction. In addition, due thereto, the staple pulley 161 of the end tool 100 may be alternately rotated in the clockwise or counterclockwise direction.

### (Actuation, Yaw, and Pitch Motions)

Actuation, yaw, and pitch motions in the present embodiment will be described as follows.

First, the actuation motion will be described below.

In a state in which a user inserts his/her index finger in the hand ring formed on the first actuation extension part 252 and his/her thumb in the hand ring formed on the second actuation extension part 257, when the user rotates the actuation rotation parts 252 and 257 using one or both of his/her index finger and thumb, the pulley 210 and the first actuation gear 253 fixedly coupled to the first actuation extension part 252 are rotated around the rotation shaft 241, and the pulley 220 and the second actuation gear 258 fixedly coupled to the second actuation extension part 257 are rotated around the rotation shaft 242. At this time, the pulley 210 and the pulley 220 are rotated in opposite directions, and thus the wires 301 and 305 fixedly coupled to the pulley 210 at one end portion thereof and the wires 302 and 306 fixedly coupled to the pulley 220 at one end portion thereof are also moved in opposite directions. In addition, a rotating force is transmitted to the end tool 100 through the power transmission part 300, and two jaws 103 of the end tool 100 perform an actuation motion.

Here, as described above, the actuation motion refers to a motion in which the two jaws 101 and 102 are splayed or closed while being rotated in opposite directions. That is, when the actuation rotation parts 252 and 257 of the actuation manipulation part 203 are rotated in directions close to each other, the first jaw 101 is rotated in the counterclockwise direction, and the second jaw 102 is rotated in the clockwise direction, thereby closing the end tool 100. That is, when the actuation rotation parts 252 and 257 of the actuation manipulation part 203 are rotated in directions away from each other, the first jaw 121 is rotated in the counterclockwise direction, and the second jaw 122 is rotated in the clockwise direction, thereby opening the end tool 100.

In the present embodiment, for the actuation manipulation described above, the first actuation extension part 252 and the second actuation extension part 257 are provided to configure the second handle and manipulated by gripping the second handle with two fingers. However, for the actuation manipulation in which the two jaws of the end tool 100 are opened or closed, the actuation manipulation part 203 may be configured in a manner different from the above-described manner, such as configuring the two actuation pulleys (the pulley 210 and the pulley 220) to act in opposition to each other with an actuation rotation part.

Next, the yaw motion will be described below.

When a user rotates the first handle 204 around the rotation shaft 243 while holding the first handle 204, the actuation manipulation part 203 and the yaw manipulation part 202 are yaw-rotated around the rotation shaft 243. That is, when the pulley 210 of the first actuation manipulation part 251 to which the wires 301 and 305 are fixedly coupled is rotated around the rotation shaft 243, the wires 301 and 305 wound around the pulleys 211 and 212 are moved. Similarly, when the pulley 220 of the second actuation manipulation part 256, to which the wires 302 and 306 are fixedly coupled, is rotated around the rotation shaft 243, the wires 302 and 306 wound around the pulleys 221 and 222 are moved. At this time, the wires 301 and 305 connected to the first jaw 101 and the wires 302 and 306 connected to the second jaw 102 are wound around the pulleys 211 and 212 and the pulleys 221 and 222, so that the first jaw 101 and the second jaw 102 are rotated in the same direction during yaw rotation. In addition, a rotating force is transmitted to the end tool 100 through the power transmission part 300, and thus a yaw motion in which two jaws 103 of the end tool 100 are rotated in the same direction is performed.

At this time, since the yaw frame 207 connects the first handle 204, the rotation shaft 241, the rotation shaft 242, and the rotation shaft 243, the first handle 204, the yaw manipulation part 202, and the actuation manipulation part 203 are rotated together around the rotation shaft 243.

Next, the pitch motion will be described below.

When a user rotates the first handle 204 around the rotation shaft 246 while holding the first handle 204, the actuation manipulation part 203, the yaw manipulation part 202, and the pitch manipulation part 201 are pitch-rotated around the rotation shaft 246. That is, when the pulley 210 of the first actuation manipulation part 251 to which the wires 301 and 305 are fixedly coupled is rotated around the rotation shaft 246, the wires 301 and 305 wound around the pulley 217 and the pulley 218 are moved. Similarly, when the pulley 220 of the second actuation manipulation part 256, to which the wires 302 and 306 are fixedly coupled, is rotated around the rotation shaft 246, the wires 302 and 306 wound around the pulley 227 and the pulley 228 are moved. At this time, as described with reference to FIG. 5, in order to allow the first jaw 101 and the second jaw 102 to pitch-rotate, the wires 301 and 305, which are first jaw wires, are moved in the same direction and respectively wound around the pulley 217 and the pulley 218, which are manipulation part pitch main pulleys, and the wires 302 and 306, which are second jaw wires, are moved in the same direction and respectively wound around the pulley 227 and the pulley 228, which are manipulation part pitch main pulleys. In addition, a rotating force is transmitted to the end tool 100 through the power transmission part 300, and two jaws 103 of the end tool 100 perform a pitch motion.

At this time, since the pitch frame 208 is connected to the yaw frame 207, and the yaw frame 207 connects the first handle 204, the rotation shaft 241, the rotation shaft 242, and the rotation shaft 243, when the pitch frame 208 is rotated around the rotation shaft 246, the yaw frame 207, the first handle 204, the rotation shaft 241, the rotation shaft 242, and the rotation shaft 243 connected to the pitch frame 208 are rotated together. That is, when the pitch manipulation part 201 is rotated around the rotation shaft 246, the actuation manipulation part 203 and the yaw manipulation part 202 are rotated together with the pitch manipulation part 201.

In summary, in the surgical instrument for electrocautery 10 according to an embodiment of the present disclosure, the pulleys are formed on respective joint points (an actuation joint, a yaw joint, and a pitch joint), the wires (the first jaw wire or the second jaw wire) are wound around the pulleys, the rotational manipulations (actuation rotation, yaw rotation, and pitch rotation) of the manipulation part cause the movement of each wire, which in turn induces the desired motion of the end tool 100. Furthermore, the auxiliary pulley may be formed on one side of each of the pulleys, and the wire may not be wound several times around one pulley due to the auxiliary pulley.

FIG. 13 is a view schematically illustrating only a configuration of pulleys and wires constituting joints of the surgical instrument for electrocautery 10 according to an embodiment of the present disclosure illustrated in FIG. 2. In FIG. 13, the relay pulleys for changing paths of the wires and not related to the operation of joints are omitted.

Referring to FIG. 13, the manipulation part 200 may include the pulley 210, the pulley 211, the pulley 212, the pulley 213, the pulley 214, the pulley 215, the pulley 216, the pulley 217, and the pulley 218 that are related to a rotational motion of the first jaw 101.

In addition, the manipulation part 200 may include the pulley 220, the pulley 221, the pulley 222, the pulley 223, the pulley 224, the pulley 225, the pulley 226, the pulley 227, and the pulley 228 that are related to a rotational motion of the second jaw 122 (the arrangement and structure of each of the pulleys of the manipulation part 200 are the same in principle as the arrangement and structure of each of the pulleys of the end tool 100, and thus specific designations of some reference numerals are omitted in the drawings).

The pulleys 211 and 212 and the pulleys 221 and 222 may be formed to be rotatable independently of each other around the same shaft, that is the rotation shaft 243. In this case, the pulleys 211 and 212 and the pulleys 221 and 222 may each be formed as two pulleys formed to face each other and formed to be independently rotatable.

The pulleys 213 and 214 and the pulleys 223 and 224 may be formed to be rotatable independently of each other around the same shaft, that is the rotation shaft 244. Here, the pulleys 213 and 214 may be formed as two pulleys formed to face each other and formed to be independently rotatable, and in this case, the two pulleys may be formed to have different diameters. Similarly, the pulleys 223 and 224 may be formed as two pulleys formed to face each other and formed to be independently rotatable, and in this case, the two pulleys may be formed to have different diameters.

The pulleys 215 and 216 and the pulleys 225 and 226 may be formed to be rotatable independently of each other around the same shaft, that is the rotation shaft 245. In this case, the pulleys 215 and 216 may be formed to have different diameters. In addition, the pulleys 225 and 226 may be formed to have different diameters.

The pulleys 217 and 218 and the pulleys 227 and 228 may be formed to be rotatable independently of each other around the same shaft, that is the rotation shaft 246.

The wire 301 is wound around the pulley 210 after sequentially passing through the pulley 217, the pulley 215, the pulley 213, and the pulley 211 of the manipulation part 200, and then is coupled to the pulley 210 by the coupling member 324. Meanwhile, the wire 305 sequentially passes through the pulley 218, the pulley 216, the pulley 214, and the pulley 212 of the manipulation part 200 and is coupled to the pulley 210 by the coupling member 324. Thus, when the pulley 210 is rotated, the wires 301 and 305 are wound around or released from the pulley 210, and accordingly, the first jaw 101 is rotated.

The wire 306 is wound around the pulley 220 after sequentially passing through the pulley 227, the pulley 225, the pulley 223, and the pulley 221 of the manipulation part 200, and then is coupled to the pulley 220 by the coupling member 327. Meanwhile, the wire 302 sequentially passes through the pulley 228, the pulley 226, the pulley 224, and the pulley 222 of the manipulation part 200 and is coupled to the pulley 220 by the coupling member 327. Thus, when the pulley 220 is rotated, the wire 302 and the wire 306 are wound around or released from the pulley 220, and accordingly, the second jaw 102 is rotated.

Thus, the actuation, yaw, and pitch manipulations are manipulatable independent of each other.

As described with reference to FIG. 1, the actuation manipulation part 203, the yaw manipulation part 202, and the pitch manipulation part 201 are configured such that the respective rotation shafts are located at the rear thereof to be identical to the joint configuration of the end tool, so that a user may intuitively perform matching manipulations.

In particular, in the surgical instrument for electrocautery 10 according to an embodiment of the present disclosure, the pulleys are formed on respective joint points (an actuation joint, a yaw joint, and a pitch joint), the wires (the first jaw wire or the second jaw wire) are formed to be wound around the pulleys, the rotational manipulations (actuation rotation, yaw rotation, and pitch rotation) of the manipulation part cause the movement of each wire, which in turn induces the desired motion of the end tool 100. Furthermore, the guide part 183 of the end tool hub 180 that functions as an auxiliary pulley may be formed on one side of the respective end tool jaw pulleys, and these auxiliary pulleys may prevent the wire from being wound on one pulley multiple times, so that the wires wound on the pulley do not come into contact with each other, and paths of the wire being wound around the pulley and the wire being released from the pulley are safely formed, so that safety and efficiency in the transmission of driving force of a wire may be improved.

Meanwhile, as described above, the yaw manipulation part 202 and the actuation manipulation part 203 are directly formed on the first handle 204. Thus, when the first handle 204 is rotated around the rotation shaft 246, the yaw manipulation part 202 and the actuation manipulation part 203 are also rotated together with the first handle 204. Accordingly, the coordinate systems of the yaw manipulation part 202 and the actuation manipulation part 203 are not fixed, but are continuously changed relative to the rotation of the first handle 204. That is, in FIG. 2 or the like, the yaw manipulation part 202 and the actuation manipulation part 203 are illustrated as being parallel to the z-axis. However, when the first handle 204 is rotated, the yaw manipulation part 202 and the actuation manipulation part 203 are not parallel to the Z-axis any longer. That is, the coordinate systems of the yaw manipulation part 202 and the actuation manipulation part 203 are changed according to the rotation of the first handle 204. However, in the present specification, for convenience of description, unless described otherwise, the coordinate systems of the yaw manipulation part 202 and the actuation manipulation part 203 are described on the basis of a state in which the first handle 204 is located perpendicular to the connection part 400 as illustrated in FIG. 2.

### <Insulation Structure of Wire>

Here, in the surgical instrument 10 according to an embodiment of the present disclosure, the wire 301 is inserted into the shrink tube 331 and the shrink tube 331 is inserted into the shaft tube 341, so that the wire 301 may not be in direct contact with the shaft tube 341 and may be insulated from the shaft tube 341.

In detail, in the case of existing surgical instruments, the wire including a metal material, which is a conductor, is directly coupled to the shaft tube including a metal material, which is also a conductor. That is, one end of the wire is inserted into the shaft tube with a hollow portion formed therein and then crimped, thereby coupling the wire to the shaft tube. Here, crimping refers to combining two or more metals or other soft materials and deforming one or both to fix the other. In this case, because the conductor is directly coupled to the conductor, current flowing through the electric wires (see 411 and 412 in FIG. 5) located around the wire and the shaft tube may leak, resulting in discharge or spark. Details thereof are described as follows.

If there is a metal (wire or shaft tube) around the electric wire through which current flows, electrical coupling may occur between the electric wire and the metal using the air filling the space between the wire and the metal. Typically, capacitive coupling or inductive coupling may occur. In particular, when high frequency/high voltage energy flows through the electric wire, an induced voltage is formed in the metal due to the above coupling.

Here, as the distance between the electric wire and the metal decreases and as the region in which the electric wire faces the metal (i.e., is adjacent to the metal and faces the metal) increases, the electrical coupling induces a strong voltage.

In addition, before a closed loop is formed by contact between a jaw of a distal end of the end tool to which the electric wire is connected, a return electrode, and body tissue of an operator, if the metal (the wire) to which voltage is induced comes into contact with body tissue, a spark may occur.

Moreover, when a voltage-induced metal touches tissue, 1) if the voltage flowing through the electric wire is high, the probability of sparking may increase, and 2) if a current density is high, sparks may occur in body tissue to burn the body tissue even though the region in which the electric wire is in contact with the metal is small so the total current is small.

In order to solve this problem, in the surgical instrument 10 according to an embodiment of the present disclosure, the shrink tube 331 is located between the wire 301 and the shaft tube 341 to reduce an overall length of the metal to which voltage may be induced. That is, the voltage induced in the section of the shaft tube 341 is blocked by the shrink tube 331 and is not transmitted to the jaws 101 and 102, and only the voltage induced in the section of the wire 301 is transferred toward the jaws 101 and 102.

Therefore, by reducing the region in which the electric wire is in contact with the metal, the amount of induced voltage may be reduced, thereby dramatically reducing the possibility of burning the body tissue even if the wire comes into contact with body tissue.

In other words, in the surgical instrument 10 according to an embodiment of the present disclosure, the wire 301 is inserted into the shrink tube 331, which is an insulator and the shrink tube 331 is inserted into the shaft tube 341 so that the wire 301 is not in direct contact with the shaft tube 341 and is insulated from the shaft tube 341, thereby improving insulation performance of the surgical instrument 10. This is described below in more detail.

As described above, one end 301a of the wire 301 is coupled to the pulley 111, which is an end tool jaw pulley. Then, the other end 301b of the wire 301 is inserted into one end 331a of the shrink tube 331. Here, the shrink tube 331 is also called a heat shrink tube, and is formed of thermoplastic plastic that changes in shape when heat is applied and processed into a straw-like shape.

An inner diameter of the shrink tube 331 is initially formed to be larger than a diameter of the wire 301 to a certain extent, and after the wire 301 is inserted into the shrink tube 331, when heat is applied to the shrink tube 331 by a heating gun or the like, the shrink tube 331 shrinks (i.e., is thermally deformed) to come into close contact with the wire 301. In other words, the shrink tube 331 is a coating material for the wire 301. As the shrink tube 331, an insulating material, such as a polyolefin shrink tube, a fluoroelastomeric tube, or a PVF tube may be used.

In this manner, after the other end 301b of the wire 301 is inserted into one end 331a of the shrink tube 331, the shrink tube 331 may be heated to be tightly coupled to the wire 301, thereby insulating the other end 301b of the wire 301, which is a conductor.

Here, the other end 301b of the wire 301 may be completely accommodated within the shrink tube 331, so that the other end 301b of the wire 301 may not protrude out of the other end 331b of the shrink tube 331. In this manner, the wire 301 may be completely physically separated and insulated from the shaft tube 341.

Meanwhile, in the shrink tube 331, the other end 331b, which is opposite to one end 331a into which the wire 301 is inserted, is inserted into the shaft tube 341. Here, the shaft tube 341 has a hollow portion formed therein and is formed in the form of an extended tube. The shaft tube 341 may be formed of, for example, steel use stainless (SUS) material.

In detail, because a length of the connection part (see 400 in FIG. 2) is relatively long, if strong tension is applied to the wire 301, the wire 301 may be stretched or sag to a certain extent within the connection part (see 400 in FIG. 2). In order to prevent the wire 301 from sagging, power may be transmitted inside the connection part (see 400 in FIG. 2) using the shaft tube 341 instead of the wire 301. That is, wires are arranged for accurate power transmission and operation within the end tool (see 100 of FIG. 2) and the manipulation part (see 200 of FIG. 2), the shaft tube 341, which is relatively thicker than the wire and less prone to deformation, is disposed in the straight part (see 401 of FIG. 2) of the connection part (see 400 of FIG. 2), and the wire on the end tool (see 100 of FIG. 2) side may be connected to the wire on the manipulation part (see 200 of FIG. 2) side by the shaft tube 341.

In the state in which the shrink tube 331 into which the wire 301 is inserted is inserted into the shaft tube 341, the shrink tube 331 and the shaft tube 341 are crimped to be coupled.

At this time, the wire 301, the shrink tube 331, and the shaft tube 341 may be located to at least partially overlap each other, and an overlapping region S may be crimped.

In this manner, the shrink tube 331, which is an insulator, is located between the wire 301, which is a conductor, and the shaft tube 341, so that the wire 301 may be completely physically separated from the shaft tube 341, without contacting, to be insulated from the shaft tube 341. Accordingly, the insulation performance of the surgical instrument 10 may be improved.

Meanwhile, the drawing shows the combination of the pulley 111, which is an end tool first jaw pulley, and the wire 301, which is a first jaw wire. However, the concept of the present disclosure is not limited thereto and may be applied equally to the wire 301 and the wire 305, which are first jaw wires, the wire 302 and the wire 306, which are second jaw wires, and the wire 303 and the wire 304, which are pitch wires.

Here, as shown, a plurality of wires are provided, and accordingly, a plurality of shrink tubes and a plurality of shaft tubes are also provided. Also, the plurality of wires and the plurality of shaft tubes may be formed not to face each other directly in any operating state. Also, to this end, the shrink tubes may be formed to have a sufficient length so that each wire and each shaft tube do not directly face each other.

In detail, each of the wire 301, 302, 303, 304, 305, and 306 moves forward or backward according to the manipulation of the manipulation part 200. At this time, a movement range of each of the wires 301, 302, 303, 304, 305 and 306 is defined to correspond to a rotation range of the jaws 101 and 102. For example, as shown in FIG. 15, the jaws 101 and 102 may rotate to a range of approximately ±120°, a movement range of the wire 301 and the wire 305, which are the first jaw wires, and the wire 302 and the wire 306, which are the second jaw wires, is determined to correspond thereto. At this time, the shrink tube is formed to have a sufficient length so that each wire and each shaft tube do not directly face each other even when one wire is fully advanced and the other wire is retreated fully.

For example, as shown in FIG. 15, even when the second jaw 102 rotates fully in the L direction, the wire 302 is retreated fully and the wire 306 is advanced fully, the shrink tube 332 may be located between the wire 302 and the shaft tube 346 so that the wire 302 and the shaft tube 346 do not directly face each other. Also, this may be applied equally between all wires, all shaft tubes and all shrink tubes.

In this manner, the plurality of wires and the plurality of shaft tubes are formed not to face each other directly in any operating state, so that the voltage induced in the section of the shaft tube 341 is not transmitted to the voltage induced in the section of the wire 301. Accordingly, by reducing the region in which the electric wire and metal face each other, the amount of induced voltage may be reduced, which dramatically reduces the possibility of burning the body tissue even if the wire comes into contact with the body tissue.

Meanwhile, a manufacturing method of the surgical instrument 10 according to an embodiment of the present disclosure is as follows.

First, the other end 301b of the wire 301 is inserted into the shrink tube 331. At this time, the wire 301 is inserted so that the other end 301b of the wire 301 does not protrude to the outside of the other end 331b of the shrink tube 331. Accordingly, the wire 301 and the shaft tube 341 may be completely physically separated and insulated.

Next, as the shrink tube 331 is heated, the shrink tube 331 and the wire 301 are tightly coupled. That is, after the wire 301 is inserted into the shrink tube 331, when heat is applied to the shrink tube 331 with a heating gun, etc., the shrink tube 331 shrinks (i.e., is thermally deformed) to be tightly coupled to the wire 301.

Next, the other end 331b of the shrink tube 331, which is opposite to one end to which the wire 301 is inserted, is inserted into the shaft tube 341. Here, the shaft tube 341 has a hollow portion formed therein and is formed in the form of a long extended tube. The shaft tube 341 may be formed of, for example, a SUS material.

Next, the wire 301, the shrink tube 331, and the shaft tube 341 are crimped together. At this time, the wire 301, the shrink tube 331, and the shaft tube 341 may be located to at least partially overlap each other, and this overlapping region S may be crimped.

In this manner, the shrink tube 331, which is a nonconductor, is located between the wire 301, which is a conductor, and the shaft tube 341, so that the wire 301 and the shaft tube 341 are completely physically separated without contacting each other, so that the wire 301 and shaft tube 341 may be insulated. Accordingly, the insulation performance of the surgical instrument 10 may be improved.

### <Modified Example of First Embodiment> (Insulation Structure of Wire)

Hereinafter, a surgical instrument for electrocautery according to a modified example of the first embodiment of the present disclosure is described. Here, the surgical instrument for electrocautery according to the modified example of the first embodiment of the present disclosure has a wire configuration different from the surgical instrument (see 10 in FIG. 2, etc.) according to the first embodiment of the present disclosure described above.

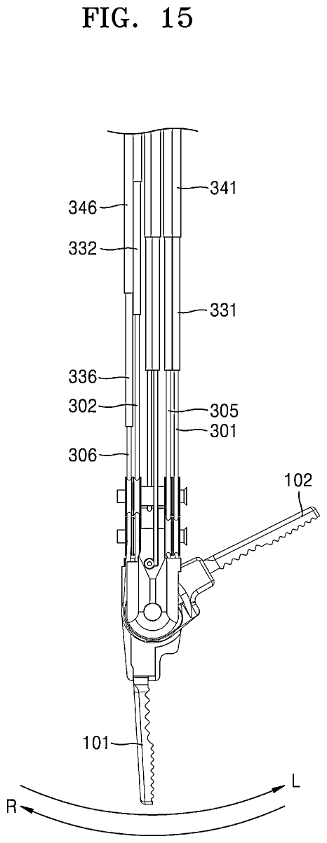

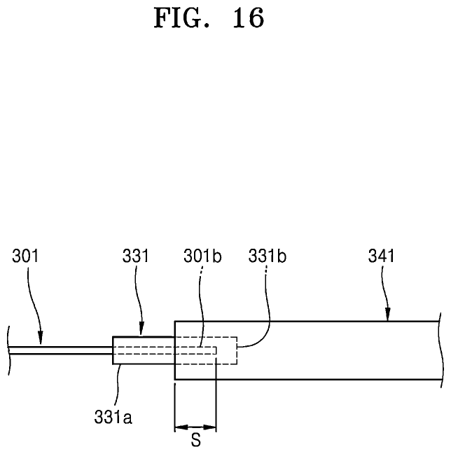

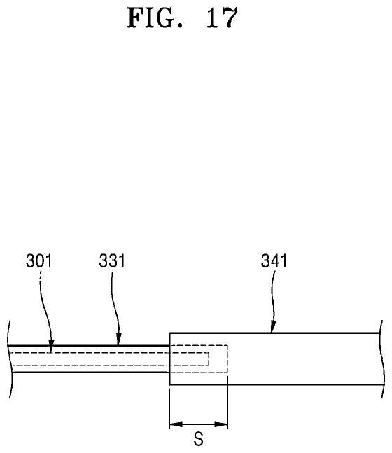

FIG. 17 is a diagram illustrating another modified example of the embodiment disclosed in FIG. 16.

In the surgical instrument 10 according to the modified example of the first embodiment of the present disclosure, the wire 301 is inserted into the shrink tube 331, which is an insulator, and the shrink tube 331 is inserted into the shaft tube 341, so that the wire 301 and the shaft tube 341 are insulated from each other not to be in direct contact with each other, thereby improving the insulation performance of the surgical instrument 10.

The shrink tube 331 may include an insulating material, such as a polytetrafluoroethylene (PTFE) tube, a polyolefin shrink tube, a fluoroelastomeric tube, or a PVF tube.

At this time, the wire 301, the shrink tube 331, and the shaft tube 341 may be located to at least partially overlap each other, and the overlapping region S may be crimped.

In this manner, the shrink tube 331, which is an insulator, is located between the wire 301, which is a conductor, and the shaft tube 341, so that the wire 301 and the shaft tube 341 are completely physically separated without contacting each other, to be insulated. Accordingly, the insulation performance of the surgical instrument 10 may be improved.

Here, the difference between the modified example and the first embodiment described above is that the shrink tube 331 is applied to the entire wire 301. In other words, the entire wire 301 is inserted into the shrink tube 331. Of course, as in the first embodiment, the end of the wire 301 inserted into the shaft tube 341 may be completely accommodated within the shrink tube 331 so that the end of the wire 301 may not protrude to the outside of the end of the shrink tube 331.

Therefore, the wire 301 has insulation performance in all operating sections of the wire 301 in the end tool 100, and the risk of electrical coupling occurring between other components including metal, such as the end tool hub 180 and the pitch hub 107 and the wire 301 may also be reduced.

Meanwhile, the drawing shows the combination of the pulley 111, which is an end tool first jaw pulley, and the wire 301, which is a first jaw wire. However, the concept of the present disclosure is not limited thereto and may be applied equally to the wire 301 and the wire 305, which are first jaw wires, the wire 302 and the wire 306, which are second jaw wires, and the wire 303 and the wire 304, which are pitch wires.

Therefore, when a plurality of wires move forward or backward according to the manipulation of the manipulation part, each wire and each shaft tube are formed not to directly face each other even when one wire is advanced fully and the other wire is retreated fully, so that the voltage induced in the section of the shaft tube 341 is not transmitted to the voltage induced in the section of the wire 301. Accordingly, by reducing the region in which the electric wire and metal face each other, the amount of induced voltage may be reduced, which dramatically reduces the possibility of burning the body tissue even if the wire comes into contact with the body tissue.

### <Second Embodiment> (Auxiliary Pulley and Hook)

Hereinafter, an end tool 500 of a surgical instrument 20 for electrocautery according to a second embodiment of the present disclosure will be described. Here, the end tool 500 of the surgical instrument for electrocautery according to the second embodiment of the present disclosure is different in the number of jaws, a configuration of an auxiliary pulley, and the like, from the end tool (see 100 of FIG. 2 and the like) of the surgical instrument according to the first embodiment of the present disclosure described above. The configuration different from the first embodiment is described in detail below.

FIGS. 18 and 19 are perspective views illustrating the end tool of a surgical instrument for electrocautery according to the second embodiment of the present disclosure. Here, FIG. 19 shows a state in which an end tool hub and a pitch hub are removed.

Referring to FIGS. 18 and 19, the end tool 500 of the second embodiment of the present disclosure may include a single jaw 501. That is, in the present embodiment, a monopolar hook, which is a one-armed cautery device, is shown as an example of a surgical instrument for electrocautery. Of course, the concept of the present disclosure is not limited thereto and may be applied to various monopolar surgical instruments for electrocautery, such as a monopolar spatula.

The end tool 500 may include a pulley 511, a pulley 512, a pulley 513, a pulley 514, a pulley 515, and a pulley 516 related to a rotational motion of the jaw 501.

Here, the drawing shows that the pulleys facing each other are formed parallel to each other, but the concept of the present disclosure is not limited thereto, and each of the pulleys may be formed in various positions and sizes suitable for the configuration of the end tool.

In addition, the end tool 500 of the second embodiment of the present disclosure may include an end tool hub 506 and a pitch hub 507.

A rotation shaft 541 and a rotation shaft 542, which are described below, may be inserted through the end tool hub 506, and the end tool hub 506 may also accommodate at least a portion of the jaw 501 axially coupled to the rotation shaft 541 therein. In addition, the end tool hub 506 may accommodate at least a portion of the pulley 512 axially coupled to the rotation shaft 542 therein.

In addition, a pulley 531 that functions as an end tool pitch pulley may be formed at one end of the end tool hub 506. As shown in FIG. 18, the pulley 531 may be formed as one body with the end tool hub 506. That is, a groove in which a wire may be wound may be formed inside the end tool hub 506. Alternatively, the pulley may be formed as a separate member from the end tool hub 506 and may be coupled to the end tool hub 506. The wire 303 and wire 304 described above are coupled to the pulley 531, which serves as an end tool pitch pulley, and the pulley 531 rotates around a rotation shaft 543 to perform a pitch motion.

A rotation shaft 543 and a rotation shaft 544, which are described below, may be inserted through the pitch hub 507 and may be axially coupled to the end tool hub 506 and the pulley 531 by the rotation shaft 543. Accordingly, the end tool hub 506 and the pulley 531 may be formed to be rotatable about the rotation shaft 543 with respect to the pitch hub 507.

In addition, the pitch hub 507 may accommodate at least a portion of the pulley 513 and the pulley 514 axially coupled to the rotation shaft 543 therein. In addition, the pitch hub 507 may accommodate at least a portion of the pulley 515 and pulley 516 axially coupled to the rotation shaft 544 therein.

In addition, the end tool 500 of the second embodiment of the present disclosure may include a rotation shaft 541, a rotation shaft 542, a rotation shaft 543, and a rotation shaft 544. As described above, the rotation shaft 541 and the rotation shaft 542 may be inserted through the end tool hub 506, and the rotation shaft 543 and the rotation shaft 544 may be inserted through the pitch hub 507.

The rotation shaft 541, the rotation shaft 542, the rotation shaft 543, and the rotation shaft 544 may be arranged sequentially in a direction from a distal end 504 to a proximal end 505 of the end tool 500. Accordingly, starting from the distal end 504, the rotation shaft 541 may be referred to as a first pin, the rotation shaft 542 may be referred to as a second pin, the rotation shaft 543 may be referred to as a third pin, and the rotation shaft 544 may be referred to as a fourth pin.

Here, the rotation shaft 541 may function as an end tool jaw pulley rotation shaft, the rotation shaft 542 may function as an end tool jaw auxiliary pulley rotation shaft, the rotation shaft 543 may function as an end tool pitch rotation shaft, and the rotation shaft 544 may function as an end tool pitch auxiliary rotation shaft of the end tool 500.

One or more pulleys may be fitted to each of the rotation shafts 541, 542, 543, and 544, which are described in detail below.

The pulley 511, which is an end tool jaw pulley, is formed to be rotatable independently of each other about the rotation shaft 541, which is an end tool jaw pulley rotation shaft. Here, the jaw 501 may be fixedly coupled to the pulley 511 and may rotate together with the pulley 511. A yaw motion of the end tool 500 is performed according to the rotation of the pulley 511. That is, the yaw motion is performed, while the pulley 511 rotates about the rotation shaft 541.

Here, the jaw 501 and the pulley 511 may be formed as separate members and coupled to each other, or the jaw 501 and the pulley 511 may be formed as a one-body.

Meanwhile, the pulley 512, which is an end tool jaw auxiliary pulley, may be additionally provided on one side of the pulley 511. In other words, the pulley 512, which is an auxiliary pulley, may be located between the pulley 511 and the pulley 513/pulley 514. The auxiliary pulley is described in more detail below.

The pulley 513 and the pulley 514 may function as end tool jaw pitch main pulleys, and the pulley 515 and the pulley 516 may function as end tool jaw pitch sub-pulleys.

Hereinafter, components related to the rotation of the pulley 511 are described.

The pulley 513 and the pulley 514 function as end tool jaw pitch main pulleys. In other words, the pulley 513 and the pulley 514 function as the main rotation pulleys for a pitch motion of the jaw 501. Here, the wire 301, which is a jaw wire, is wound around the pulley 513, and the wire 305, which is a jaw wire, is wound around the pulley 514.

The pulley 515 and the pulley 516 function as end tool jaw pitch sub-pulleys. In other words, the pulley 515 and the pulley 516 function as sub-rotation pulleys for a pitch motion of the jaw 501. Here, the wire 301, which is a jaw wire, is wound around the pulley 515, and the wire 305, which is a jaw wire, is wound around the pulley 516.

Here, the pulley 513 and the pulley 514 are arranged to face each other on one side of the pulley 511 and the pulley 512. Here, the pulley 513 and the pulley 514 are formed to be rotatable independently of each other about the rotation shaft 543, which is an end tool pitch rotation shaft. In addition, the pulley 515 and the pulley 516 are arranged to face each other on one side of the pulley 513 and the pulley 514, respectively. Here, the pulley 515 and the pulley 516 are formed to be rotatable independently of each other about the rotation shaft 544, which is an end tool pitch auxiliary rotation shaft. Here, in the drawing, the pulley 513, the pulley 515, the pulley 514, and the pulley 516 are all shown as being rotatable in the Y-axis direction, but the concept of the present disclosure is not limited thereto, the rotation shaft of each pulley may be formed in various directions to suit a configuration thereof.

The wire 301, which is a jaw wire, is sequentially wound so that at least a portion thereof contacts the pulley 515, the pulley 513, and the pulley 511. Also, the wire 305 connected to the wire 301 by the fastening member 323 is sequentially wound so that at least a portion thereof contacts the pulley 511, the pulley 512, the pulley 514, and the pulley 516.

From another perspective, the wire 301 and wire 305, which are jaw wires, are formed to be sequentially wound to at least partially contact the pulley 515, the pulley 513, the pulley 511, pulley 512, the pulley 514, and the pulley 516, and the wire 301 and the wire 305 are formed to move along the pulleys, while rotating the pulleys.

Accordingly, when the wire 301 is pulled, the fastening member 323 to which the wire 301 is coupled and the pulley 511 coupled thereto rotate counterclockwise. Conversely, when the wire 305 is pulled, the fastening member 323 to which the wire 305 is coupled and the pulley 511 coupled thereto rotate clockwise.

Hereinafter, the pulley 512, which serves as an auxiliary pulley, is described in more detail.

The pulley 512 may serve to increase a rotation angle of the jaw 501 by coming into contact with the wire 305, which is a jaw wire, to change an arrangement path of the wire 305. That is, when the auxiliary pulley is not disposed, the jaw may rotate up to a right angle, but in an embodiment of the present disclosure, by additionally providing the pulley 512, which is an auxiliary pulley, the maximum rotation angle may increase.

This is described below in more detail.

When the auxiliary pulley is not disposed, the jaw wire is fixedly coupled to the end tool jaw pulley, so the end tool jaw pulley may rotate only up to 900 in the left and right direction. Meanwhile, in the case of the surgical instrument 20 for electrocautery of the present disclosure, the pulley 512, which is an auxiliary pulley, is additionally disposed on one side of the pulley 511. By changing the arrangement path of the wire 305, which is a jaw wire, to a certain extent by disposing the pulley 512 as described above, a tangential direction of the wire 305 is changed, and thus, the fastening member 326 that couples the wire 305 to the pulley 511 may rotate up to about 120°. That is, the fastening member 326, which is a coupling part of the wire 305 and the pulley 511, is rotatable until the fastening member 326 is located on the common internal tangent of the pulley 511, thereby expanding a rotation range in both directions.

From another perspective, the wire 301 and wire 305, which are two strands of the jaw wire wound around the pulley 511, are disposed on one side based on a plane perpendicular to the Y axis and passing through the X axis, by the pulley 512. From another perspective, the pulley 513 and the pulley 514 may be disposed on one side based on the plane perpendicular to the Y-axis and passing through the X-axis. From another perspective, the wire 305 is located on the internal tangent of the pulley 511 and the pulley 512, and the rotation angle of the pulley 511 is increased by the pulley 512.

According to the present disclosure, a rotation radius of the jaw 501 may be increased.

### <Third Embodiment> (End Tool Hub/Pitch Hub)

Hereinafter, an end tool 600 of a surgical instrument 30 for electrocautery according to the third embodiment of the present disclosure is described. Here, the end tool 600 of the surgical instrument 30 for electrocautery according to the third embodiment of the present disclosure is different in a configuration of an end tool hub 605 and a pitch hub 607 from the end tool (see 100 of FIG. 4 or the like) of the surgical instrument for electrocautery according to the first embodiment of the present disclosure described above.

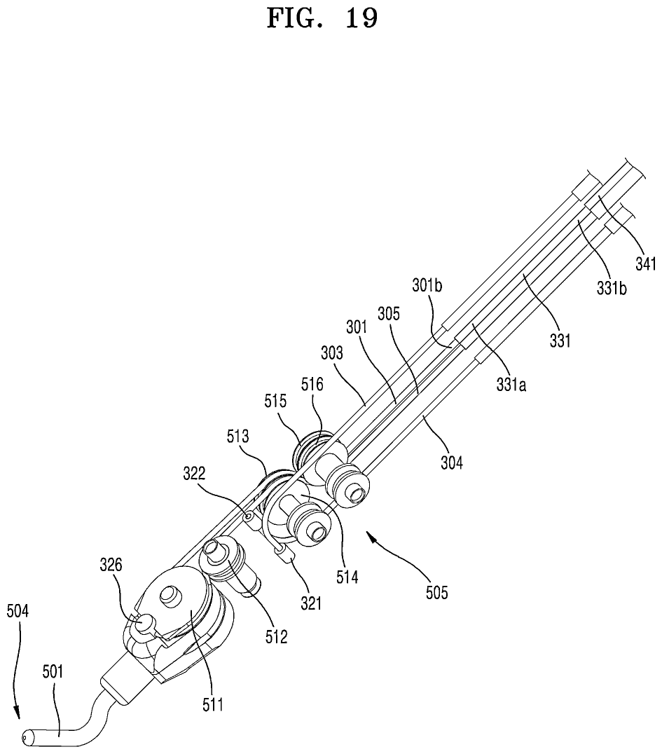

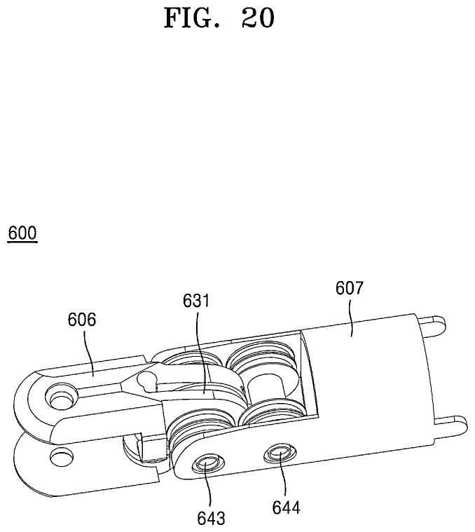

FIG. 20 is a perspective view illustrating the end tool hub 606 and the pitch hub 607 of the surgical instrument 30 for electrocautery according to the third embodiment of the present disclosure.

Common contents, such as a structure and coupling relationship of the end tool hub 606 and the pitch hub 607 are the same as described above in the description of the surgical instrument for electrocautery according to the first embodiment, so only different parts are described here.

Because the end tool hub 606 and the pitch hub 607 are formed of a metal material, current flowing through the electric wire passing through inside the end tool hub 606 and the pitch hub 607 may leak or electrical bonding may occur between the electric wire and the metal.

Therefore, apart from the insulation of the wire described above, there is a need to insulate the end tool hub 606 and the pitch hub 607 to prevent electrical coupling in the end tool.

That is, the surface of the end tool hub 606 and the pitch hub 607 formed of metal may be coated with a material having insulating properties so that the end tool hub 606 and the pitch hub 607 have an insulating structure.

In this manner, ceramics, parylene, etc. may be used as insulating materials for coating the metal surface, and examples of ceramic coating materials include alumina, fused silica, and magnesia. The following various methods may be used to coat metal with ceramic. For example, various deposition methods, such as enamel coating, thermal spray coating, physical vapor deposition (PVD), and chemical vapor deposition (CVD) may be used.

### <Fourth Embodiment> (Insulated Pulley)

Hereinafter, an end tool 700 of a surgical instrument 40 for electrocautery according to a fourth embodiment of the present disclosure is described. Here, the end tool 700 of the surgical instrument 40 for electrocautery according to the fourth embodiment of the present disclosure is different in a configuration of an end tool hub and a pulley from the end tool (see 100 of FIG. 4 and the like) of the surgical instrument for electrocautery according to the first embodiment of the present disclosure described above.

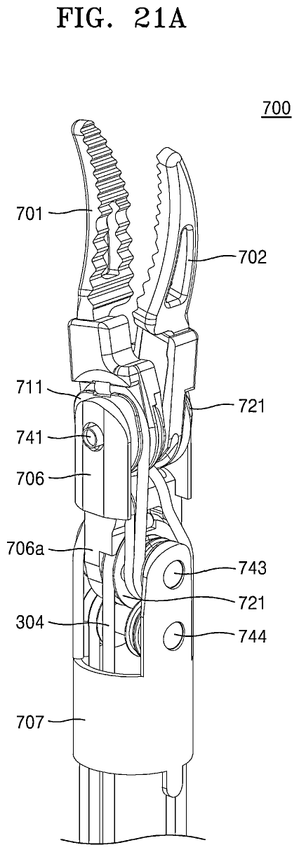

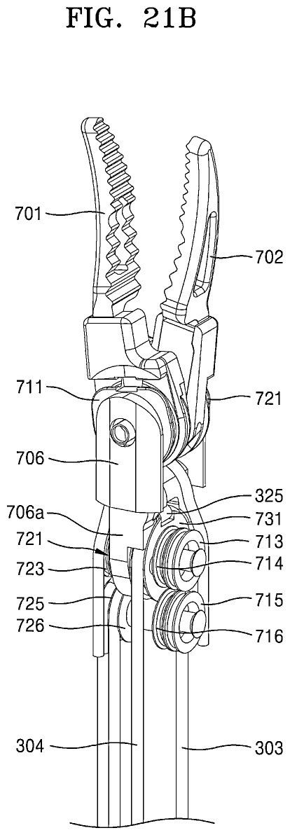

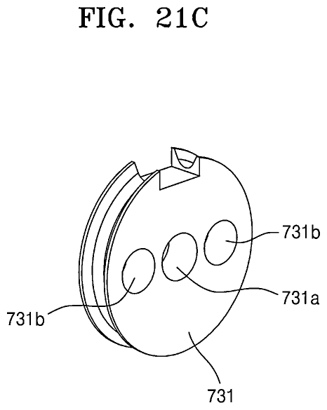

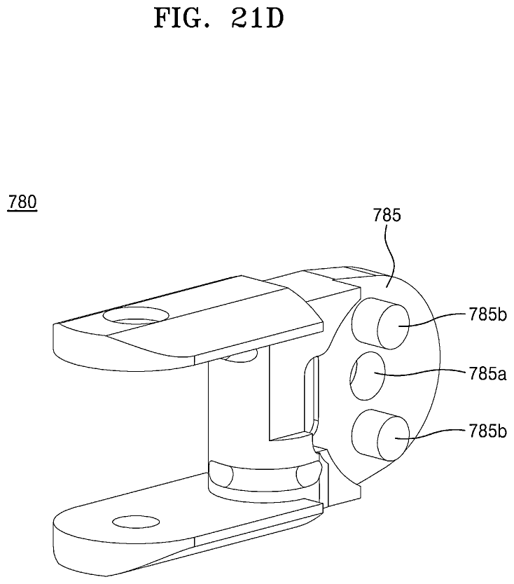

FIGS. 21A and 21B are perspective views illustrating an end tool of a surgical instrument for electrocautery according to a fourth embodiment of the present disclosure, FIG. 21C is a perspective view illustrating an insulating pulley of the end tool shown in FIGS. 21A and 21B, and FIG. 21D is a perspective view illustrating an end tool hub shown in FIGS. 21A and 21B. Here, FIG. 21B shows a state in which the pitch hub is removed.

Referring to FIGS. 21A and 21B, the end tool 700 of the fourth embodiment of the present disclosure may include a pair of jaws, that is, a first jaw 701 and a second jaw 702. The end tool 700 may include a pulley 711, a pulley 713, a pulley 714, a pulley 715, and a pulley 716 related to a rotational motion of the first jaw 701. It may also include pulley 721, pulley 723, pulley 724, pulley 725, and pulley 726 related to the rotational motion of the second jaw 702.

In addition, the end tool 700 of the first embodiment of the present disclosure may include an end tool hub 780 and a pitch hub 707. The end tool hub 780 is described in more detail below.

In addition, the end tool 700 of the fourth embodiment of the present disclosure may include a rotation shaft 741, a rotation shaft 743, and a rotation shaft 744. The rotation shaft 741 may be inserted through the end tool hub 780, and the rotation shaft 743 and the rotation shaft 744 may be inserted through the pitch hub 707.

In the fourth embodiment of the present disclosure, the first jaw 701 and the second jaw 702, the pitch hub 707, each pulley and each rotation shaft, etc., are substantially the same as the end tool 100 described above with reference to FIG. 4 or the like, and thus, a detailed description thereof is omitted here.

Hereinafter, the end tool hub 780 of the fourth embodiment of the present disclosure is described in more detail, and a pulley 731 formed at one end of the end tool hub 780 and serving as an end tool pitch pulley is mainly described.

A rotation shaft 741, which is to be described below, is inserted through the end tool hub 780, and a pulley 711 and a pulley 721 axially coupled to the rotation shaft 741 and at least portions of the first jaw 701 and second jaw 702 coupled thereto may be accommodated within the end tool hub 780. Here, in the fourth embodiment of the present disclosure, the pulley 731 that functions as an end tool pitch pulley is formed in the end tool hub 780.

Here, the pulley 731 is formed as a separate member from the end tool hub 780 and may be coupled to the end tool hub 780. That is, the pulley 131 of the end tool hub 180 shown in FIG. 4 represents an example formed as one body with the end tool hub, but as shown in FIGS. 21A and 21B, the pulley 731 of the end tool hub 780 according to the fourth embodiment of the present disclosure may be an independent component that may be separated and distinguished from the end tool hub 780.

In detail, the end tool hub 780 may include a pitch pulley part 785 formed in a direction opposite to the direction in which the jaw pulley coupling part coupled to the jaw is formed. Also, a pulley 731, which is a pitch pulley on which the wire 303 and wire 304, which are pitch wires, may be wound, may be formed in the pitch pulley part 785. Here, the pulley 731 is a member that rotates about an axis and may function as an end tool pitch pulley. Here, the pitch pulley part 785 may be formed on the XZ plane. In addition, a through-hole through which the rotation shaft 743 may be inserted may be formed in the pitch pulley part 785. That is, the pitch pulley part 785 and the pulley 731 may be formed as separate members, and the rotation shaft 743 may pass through the pitch pulley part 785 and the pulley 731 to couple the two members.

Meanwhile, the pulley 731 may include at least one coupling part that fixedly couples the pulley 731 to the end tool hub 780. For example, the pulley 731 may be further formed with one or more through-holes 731b in addition to a through-hole 731a through which the rotation shaft 743 may be inserted. In addition, the pitch pulley part 785 may be formed with a protrusion 785b protruding in the direction of the pulley 731 on one surface in contact with the pulley 731, and the protrusion 785b may be coupled to a through-hole 731b. That is, as the protrusion 785b formed on the pitch pulley part 785 is coupled to the through-hole 731b of the pulley 731, the end tool hub 780 may also rotate together when the pulley 731 rotates about the rotation shaft 743. For example, the through-hole 731a into which the rotation shaft 743 is inserted is formed in the center of the pulley 731, two through-holes 731b are additionally formed, and the additional through-hole 731b is coupled to the protrusion 785b of the pitch pulley part 785 to transmit the rotation of the pulley 731 to the pitch pulley part 785. However, the concept of the present disclosure is not limited thereto, and the number, location, and size of through-holes may be vary. In addition, of course, a protrusion may be formed on the pulley 731 and a through-hole, which is a coupling part, may also be formed on the pitch pulley part 785.

Continuing to describe the pulley 731, the pulley 731 of the fourth embodiment of the present disclosure has insulating properties. That is, the pulley 731 serving as the end tool pitch pulley may include an insulator, and the end tool hub 780 and the fastening member 325 may be separated by the pulley 731 having insulating properties, so that the end tool hub 780 may be insulated from the fastening member 325.

In detail, the fastening member 325 formed of metal is coupled to the pulley 731 having insulating properties, and the pulley 731 is coupled to the end tool hub 780 formed of metal. That is, the fourth embodiment of the present disclosure is different from the previous embodiments, in that the fastening member 325 of the pitch wire does not directly contact the end tool hub 780 formed of metal but the insulating pulley 731 is located between the end tool hub 780 and the fastening member 325 so that the metal fastening member 325 is completely physically separated from the metal end tool hub 780.

In this manner, because the insulating pulley 731 is located between the end tool hub 780 and the fastening member 325, the occurrence of sparks when the fastening member 325 formed of metal coupled to the end of the pitch wire comes into contact with the end tool hub 780 formed of metal may be prevented.

The insulating pulley 731 may be formed of plastic, ceramic, etc. For example, the pulley 731 may be formed of polytheretherketone (PEEK), phenolic resin, polycarbonate, or glass fiber reinforced plastic (GRP) or may be plastic coated with ceramic.

Meanwhile, the wire 303 and the wire 304 are coupled to the pulley 731, which serves as an end tool pitch pulley, and the pulley 731 rotates about the rotation shaft 743 to perform a pitch motion.

Here, the wire 303 and the wire 304 form a pair and may function as a pitch wire. In addition, a fastening member 325 coupled to the end of the wire may be included to couple the wire to the pulley. Here, each fastening member may have various shapes depending on need, such as a ball shape or a tube shape.

The wire 303 and the wire 304, which are pitch wires, may be a single wire. After inserting the fastening member 325 into a middle point of the pitch wire, which is a single wire, and fixing the fastening member 325 by crimping, both strands of the pitch wire may be referred to as the wire 303 and the wire 304, respectively.

Alternatively, the wire 303 and the wire 304, which are pitch wires, may each be formed as separate wires, and the wire 303 may be connected to the wire 304 by the fastening member 325.

Also, by coupling the fastening member 325 to the pulley 731, the wire 303 and the wire 304 may be fixedly coupled to the pulley 731. As a result, the pulley 731 may rotate as the wire 303 and the wire 304 are pulled and released.

The rotation shaft 743 and the rotation shaft 744 may be inserted through the pitch hub 707, and the pitch hub 707 may be axially coupled to the end tool hub 780 and the pulley 731 by the rotation shaft 743. Accordingly, the end tool hub 780 and the pulley 731 may be formed to be pitch-rotated with respect to the pitch hub 707 based on the rotation shaft 743.

### <Fifth Embodiment> (Merging Wire and Electric Wire)

Hereinafter, a surgical instrument for electrocautery according to a fifth embodiment of the present disclosure will be described. Here, the surgical instrument for electrocautery according to the fifth embodiment of the present disclosure is different in a wire configuration from the surgical instrument (see 10 of FIG. 2 or the like) according to the first embodiment of the present disclosure described above. The configuration different from the first embodiment is described in detail below.

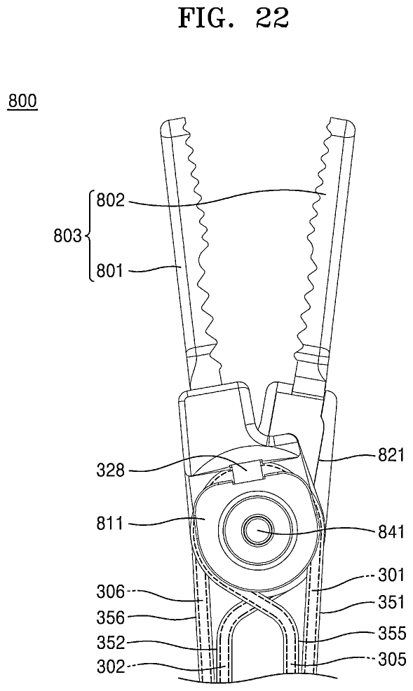

FIG. 22 is a diagram illustrating a jaw pulley and a wire connection configuration in an end tool of a surgical instrument for electrocautery according to a fifth embodiment of the present disclosure.

Referring to FIG. 22, an end tool 800 of the surgical instrument for electrocautery according to the fifth embodiment of the present disclosure includes a pair of jaws that is, a first jaw 801 and a second jaw 802, for performing a grip operation. Here, the first jaw 801 and the second jaw 802 separately or a component encompassing the first jaw 801 and the second jaw 802 may be referred to as a jaw 803.

The pulley 811 functions as an end tool first jaw pulley, and the pulley 821 functions as an end tool second jaw pulley. The pulley 811 may be referred to as a first jaw pulley, and the pulley 821 may be referred to as a second jaw pulley, and these two components may be collectively referred to as an end tool jaw pulley.

The pulley 811 and pulley 821, which are end tool jaw pulleys, are formed to face each other and are formed to rotate independently of each other about the rotation shaft 841, which is an end tool jaw pulley rotation shaft. Here, in the drawing, the pulley 811 and the pulley 821 are formed to rotate about one rotation shaft 841, but of course, each pulley may be formed to rotate about a separate shaft. Here, the first jaw 801 is fixedly coupled to the pulley 811 and rotates together with the pulley 811, and the second jaw 802 is fixedly coupled to the pulley 821 and rotates together with the pulley 821. A yaw motion and an actuation motion of the end tool 800 are performed according to the rotation of the pulley 811 and the pulley 821. That is, the yaw motion is performed when the pulley 811 and the pulley 821 rotate in the same direction about the rotation shaft 841, and the actuation motion is performed when the pulley 811 and the pulley 821 rotate in opposite directions about the rotation shaft 841.

Meanwhile, a power transmission part 300 of the surgical instrument for electrocautery according to the fifth embodiment of the present disclosure includes the wire 301, the wire 302, the wire 303, the wire 304, the wire 305, and the wire 306.

Here, the wire 301 and the wire 305 may form a pair and serve as a first jaw wire. The wire 302 and the wire 306 may form a pair and serve as a second jaw wire. Here, the components encompassing the wire 301 and the wire 305, which are first jaw wires, and the wire 302 and the wire 306, which are second jaw wires, may be referred to as jaw wires. In addition, the wire 303 and the wire 304 may form a pair and serve as a pitch wire.

Here, in the instrument 50 for electrocautery surgery according to the fifth embodiment of the present disclosure, the wire is covered with an electric wire coating. In other words, the entire wire may be wrapped with an insulating material and formed as an electric wire.

In detail, the wire 301 is covered with an electric wire coating and may be referred to as an electric wire 351, the wire 302 may be referred to as an electric wire 352, the wire 305 may be referred to as an electric wire 355, and the wire 306 may be referred to as an electric wire 356.

In the surgical instrument for electrocautery 10 of the first embodiment of the present disclosure, the wires serve to transmit power from the manipulation part 200 to the end tool 100, and electrical energy is transmitted from the connector 410 to the jaw 103 through separate electric wires 411 and 412.

Meanwhile, in the surgical instrument 50 for electrocautery of the fifth embodiment of the present disclosure, the wire may transmit power from the manipulation part 200 to the end tool 100 and simultaneously transmit electrical energy from the connector 410 to the jaw 803. Specifically, the wire 301 and the 305, which are first jaw wires, and the wire 302 and the wire 306, which are second jaw wires, may be formed as the electric wire 351, the electric wire 355, the electric wire 352, and the electric wire 356, respectively, and the electric wire coating may be formed of a material, for example, perfluoroalkoxy alkanes (PFA).

Meanwhile, the first jaw 801 may be formed of a conductor, such as metal, and connected to an electric wire. In other words, the electric wire 351 may include a region from which the coating has been removed (a fastening region, not shown) at an end coupled to the first jaw 801, and the region from which the coating has been removed may be in contact with the first jaw 801 and transfer electrical energy to the first jaw 801, which is a conductor. From another perspective, the electric wire 351 may be considered to be covered and wound around the pulley 811, except for the portion thereof coupled to the first jaw 801. At this time, the pulley 811 may be formed of a nonconductor, such as ultem, ceramic, or plastic.

In other words, the electric wire 351 is wound around the pulley 811, which is an insulator, and the fastening region (not shown) at the end of the electric wire 351 is coupled to the first jaw 801, which is a conductor. That is, the first jaw 801 to which the electric wire 351 is connected is coupled to the pulley 811, and the electric wire 351 is wound around the pulley 811, which is an insulator, and extends toward the manipulation part (not shown).

Meanwhile, the electric wire 355 may also include a region (a fastening region, not shown) from which the coating has been removed at an end coupled to the first jaw 801, and the region from which the coating has been removed may be in contact with the first jaw 801 and transfer electrical energy to the first jaw 801, which is a conductor. From another perspective, the electric wire 355 may be considered to be covered and wound around the pulley 811, except for the portion thereof coupled to the first jaw 801. The first jaw 801 to which the electric wire 355 is connected is coupled to the pulley 811, and the electric wire 355 is wound around the pulley 811, which is an insulator, and extends toward the manipulation part (not shown).

Alternatively, the electric wire 351 and the electric wire 355, which are electric wires coupled to the first jaw, may be a single electric wire. That is, after the coating at the middle point of the single electric wire is peeled off and the fastening member 328 is inserted into the peeled portion and then crimped to be fixed, both strands of the electric wire based on the fastening member may be referred to as the electric wire 351 and the electric wire 355, respectively.

Similarly, the second jaw 802 may be formed of a conductor, such as metal, and connected to an electric wire. In other words, the electric wire 352 may include a region (the fastening region, not shown) from which the coating has been removed at the end coupled to the second jaw 802, and the region from which the coating has been removed is in contact with the second jaw 802 to transfer electrical energy to the second jaw 802, which is a conductor. From another perspective, the electric wire 352 may be considered to be covered and wound around the pulley 821, except for the portion thereof coupled to the second jaw 802. Here, the pulley 821 may be formed of a nonconductor, such as ultem, ceramic, or plastic.

In other words, the electric wire 352 is wound around the pulley 821, which is an insulator, and the fastening region (not shown) at the end of the electric wire 352 is coupled to the second jaw 802, which is a conductor. That is, the second jaw 802 to which the electric wire 352 is connected is coupled to the pulley 821, and the electric wire 352 is wound around the pulley 821, which is an insulator, and extends toward the manipulation part (not shown).

Meanwhile, the electric wire 356 may also include a region (fastening region, not shown) from which the coating has been removed at the end coupled to the second jaw 802, and the region from which the coating has been removed may be in contact with the second jaw 802 and transfer electrical energy to the second jaw 802, which is a conductor. From another perspective, the electric wire 356 may be considered to be covered and wound around the pulley 821, except for the portion thereof coupled to the second jaw 802. The second jaw 802 to which the electric wire 356 is connected is coupled to the pulley 821, and the electric wire 356 is wound around the pulley 821, which is an insulator, and extends toward the manipulation part (not shown).

By changing the wire to an electric wire as described above, the wire may perform both the power transmission function and the electric energy transmission function at the same time, and because there is no need to have a separate electric wire or a pulley around which an electric wire is wound, the overall configuration may be reduced and the process may be simplified.

### <Sixth Embodiment> (Monopolar Dissector)

Hereinafter, an end tool 1100 of a surgical instrument 60 for electrocautery according to the sixth embodiment of the present disclosure is described. Here, the end tool 1100 of the surgical instrument for electrocautery according to the sixth embodiment of the present disclosure is different in configuration from the end tool (see 100 of FIG. 2 or the like) of the surgical instrument according to the first embodiment of the present disclosure described above, in that the shape of the jaw is different and a cover and a relay tube is added. The configuration different from the first embodiment is described in detail below.

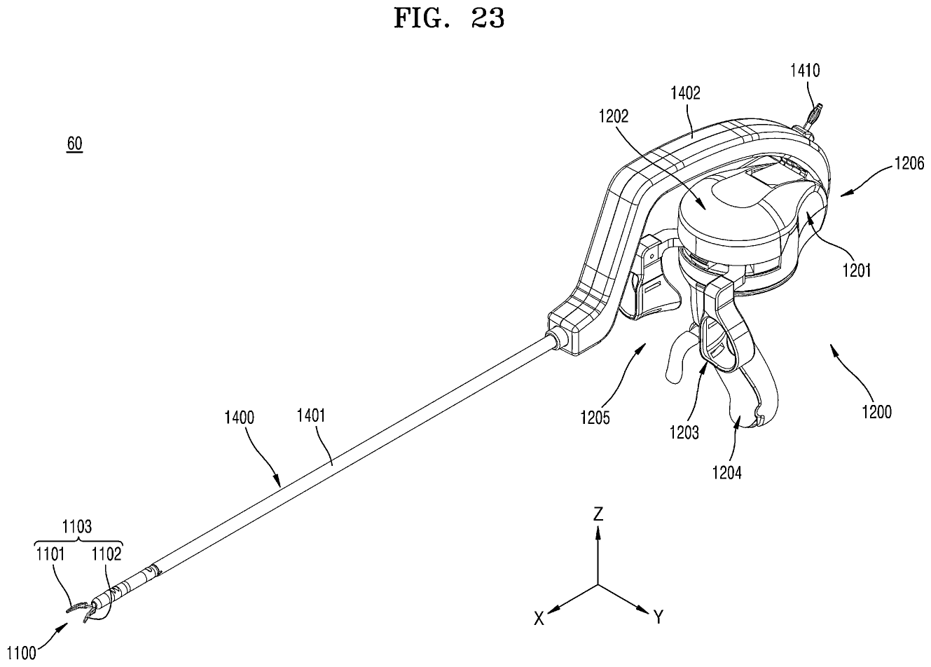

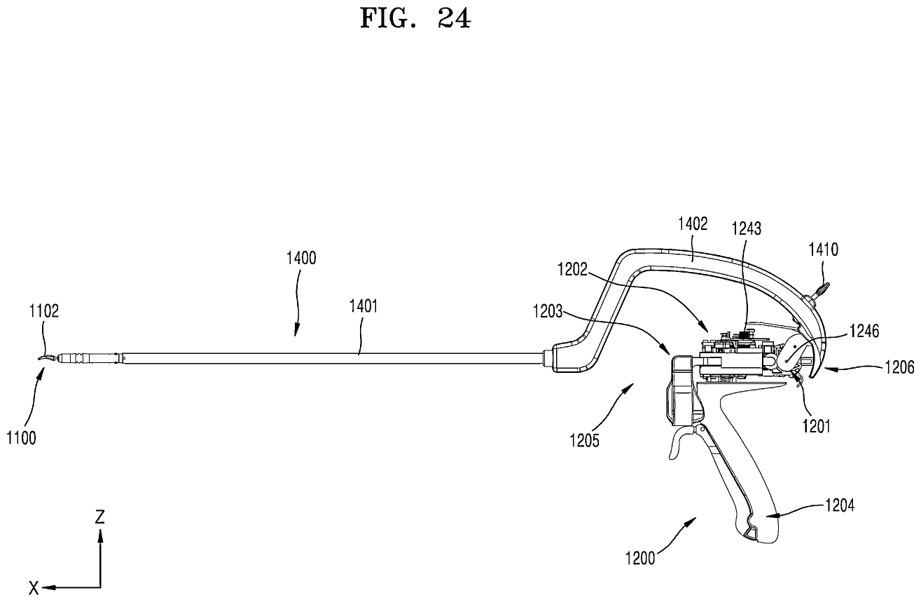

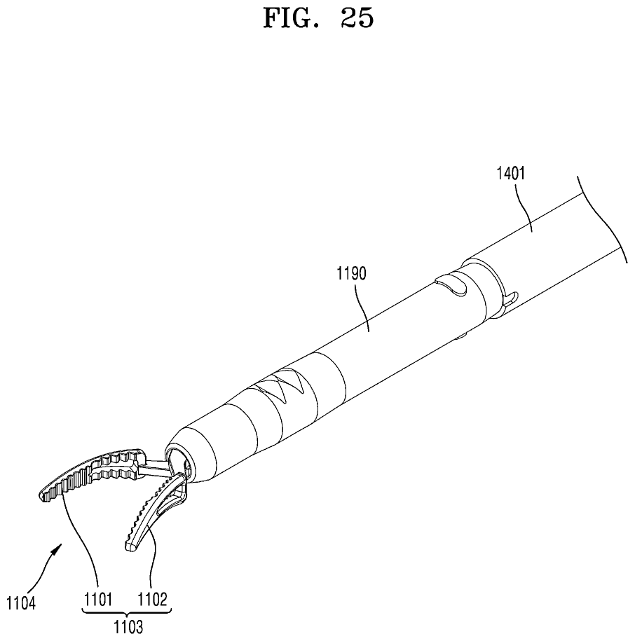

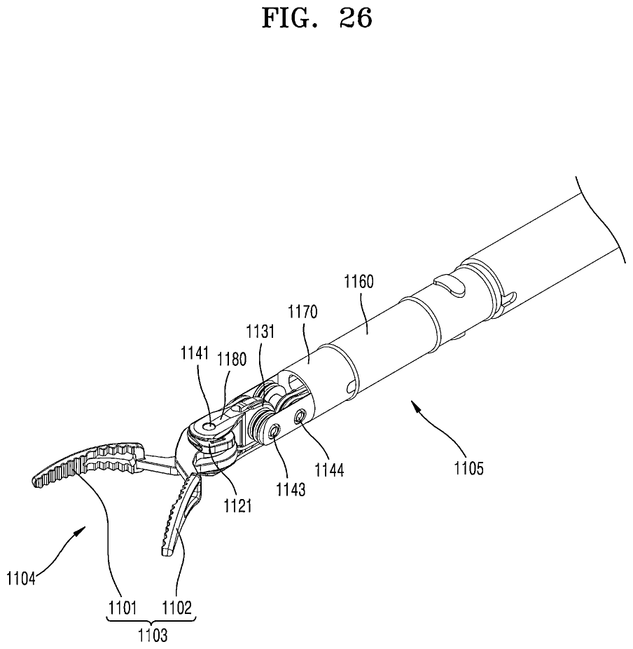

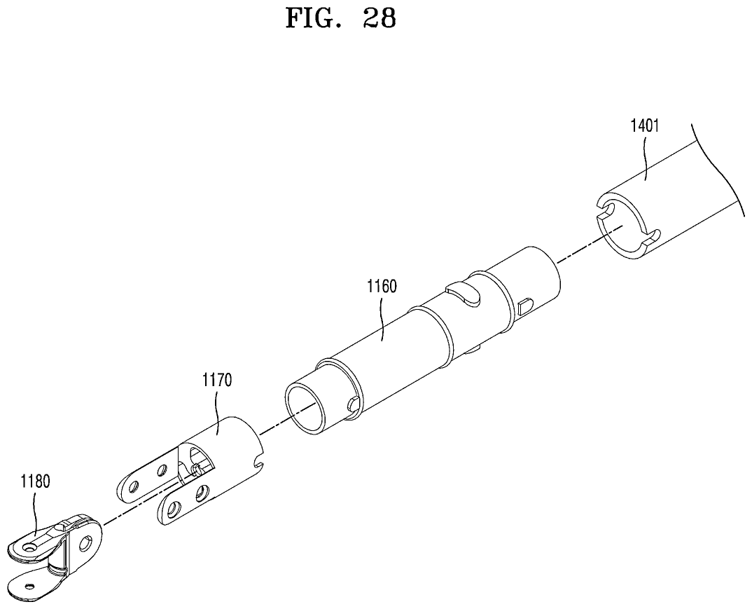

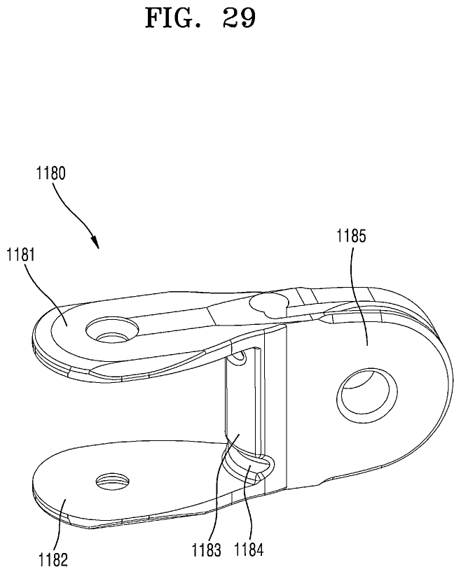

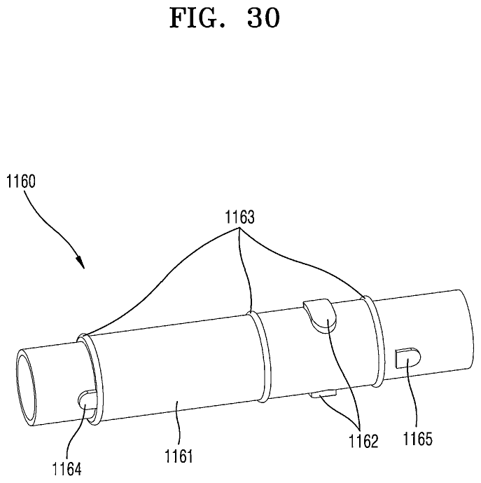

FIG. 23 is a perspective view illustrating a surgical instrument for electrocautery according to a sixth embodiment of the present disclosure, and FIG. 24 is a side view of the surgical instrument for electrocautery of FIG. 23. FIGS. 25 to 27 are perspective views illustrating an end tool of the surgical instrument for electrocautery of FIG. 23, FIG. 28 is an exploded perspective view illustrating an end tool hub, a pitch hub, a relay tube, and a shaft shown in FIG. 26, FIG. 29 is an enlarged perspective view illustrating the end tool hub of the surgical instrument for electrocautery of FIG. 26, and FIG. 30 is an enlarged perspective view illustrating the relay tube of the surgical instrument for electrocautery of FIG. 26.

First, referring to FIGS. 23 and 24, the instrument 60 for electrocautery surgery according to the sixth embodiment of the present disclosure includes the end tool 1100, a manipulation part 1200, a power transmission part (not shown), and a connection part 1400.

The connection part 1400 includes a straight part 1401 and a bent part 1402 and includes a connector 1410. The manipulation part 1200 includes a first handle 1204, an actuation manipulation part 1203, a yaw manipulation part 1202, and a pitch manipulation part 1201.

In the present embodiment, each component of the connection part 1400 and the manipulation part 1200 are substantially the same as the connection part 400 and the manipulation part 200 described above with reference to FIG. 2 and the like in the first embodiment, so a detailed description thereof is omitted here.

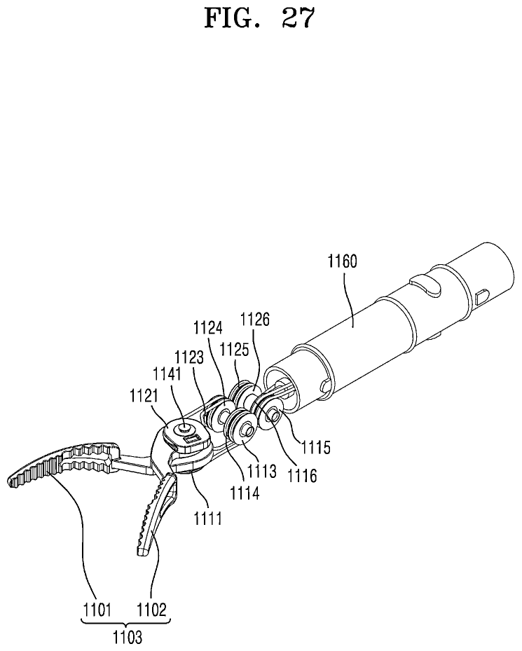

FIG. 25 is a perspective view illustrating the end tool 1100 provided with a cover 1190, FIG. 26 is a perspective view illustrating a state in which the cover 1190 is removed from the end tool 1100 of FIG. 25, and FIG. 27 is a perspective view illustrating an end tool hub 1180 and a pitch hub 1170 are removed from the end tool 1100 of FIG. 26.

Referring to FIGS. 25 to 27, the end tool 1100 of the surgical instrument 60 for electrocautery according to the sixth embodiment of the present disclosure includes a pair of jaws, that is, the first jaw 1101 and the second jaw 1102. Here, the first jaw 1101 and the second jaw 1102 separately or a component encompassing the first jaw 1101 and the second jaw 1102 may be referred to as a jaw 1103. The first jaw 1101 and the second jaw 1102 are described in detail below.

Meanwhile, the end tool 1100 may include a pulley 1111, a pulley 1113, a pulley 1114, a pulley 1115, and a pulley 1116 related to a rotational motion of the first jaw 1101. In addition, the end tool 1100 may include a pulley 1121, a pulley 1123, a pulley 1124, a pulley 1125, and a pulley 1126 related to the rotational motion of the second jaw 1102. In the present embodiment, the pulleys related to the rotational motion of the first jaw and the pulleys related to the rotational motion of the second jaw are substantially the same as the pulleys 1111, 1113, 1114, 1115, 1116, 1121, 1123, 1124, 1125, and 1126), and thus, a detailed description therof is omitted here.

In addition, the end tool 1100 may include an end tool hub 1180, a pitch hub 1170, and a relay tube 1160. These components may be sequentially coupled in the direction from a distal end 1104 to a proximal end 1105 of the end tool, and the relay tube 1160 may be connected to a shaft, which is a straight part 1401 of the connection part 1400. Here, the cover 1190 and the relay tube 1160 are described in detail below.

Meanwhile, although not shown in the drawings, the surgical instrument 60 for electrocautery according to the sixth embodiment of the present disclosure may further include the wire (see 301 of FIG. 16), the shrink tube (see 331 of FIG. 16), and a shaft tube (see 341 of FIG. 16), like the surgical instrument for electrocautery 10 according to the first embodiment of the present disclosure shown in FIGS. 2 and 16 and the like. In addition, in the surgical instrument 60 according to the sixth embodiment of the present disclosure, the wire (see 301 of FIG. 16) is inserted into the shrink tube (see 331 of FIG. 16) and the shrink tube (see 331 in FIG. 16) is inserted into the shaft tube (see 341 of FIG. 16), so that the wire (see 301 of FIG. 16) and the shaft tube (see 341 of FIG. 16) are not in direct contact but are insulated from each other.

In the end tool 1100 of the sixth embodiment of the present disclosure, a rotation shaft 1141 is inserted through the end tool hub 1180, a rotation shaft 1143 and a rotation shaft 1144 are inserted through the pitch hub 1170, and the pitch hub 1170 may be axially coupled to the end tool hub 1180 and the pulley 1131 by the rotation shaft 1143. Accordingly, the end tool hub 1180 and the pulley 1131 may be formed to be pitch-rotated with respect to the pitch hub 1170 based on the rotation shaft 1143.

In the present embodiment, the end tool hub 1180, the pitch hub 1170, and each of the rotation shafts 1141, 1143, and 1144 are substantially the same as the end tool hub 1180, the pitch hub 1107, and each of the rotation shafts 1141, 1143, and 1144 described above with reference to FIG. 4 or the like of the first embodiment, and thus, a detailed description thereof is omitted here.

Meanwhile, the end tool 1100 of the sixth embodiment of the present disclosure may further include the relay tube 1160. The relay tube 1160 may be formed of an insulating material. For example, the relay tube 1160 may be formed of polyether ether ketone (PEEK), a phenolic resin, polycarbonate, or glass fiber reinforced plastic (GRP), and may be plastic coated with ceramic. The relay tube 1160 may be coupled to one end of the pitch hub 1170 described above and may be coupled to the straight part 1401 of the connection part 1400. That is, the relay tube 1160 may be a member located between the straight part 1401 and the pitch hub 1170.

In detail, the relay tube 1160 may be located between the pitch hub 1170, which is a conductor, and the straight part 1401, which is a conductor, and may serve as an insulating adapter, and may serve to fix the cover 1190, which is to be described below.

This is described below in more detail.

The relay tube 1160 may include one or more hub coupling parts 1164 coupled to the pitch hub 1170 at one end of the tube body 1161 and one or more shaft coupling parts 1165 coupled to the straight part 1401 of the connection part 1400 at the other end of the tube body 1161. In addition, a cover coupling part 1162 coupled to the cover 1190 may be formed in a region of the tube body 1161. In detail, one end of the tube body 1161 is a portion inserted into an inner surface of the pitch hub 1170 and may have a diameter less than that of the center of the body, and the other end of the tube body 1161 is a portion inserted into an inner surface of the straight part 1401 of the connection part 1400 and may have a diameter less than that of the center of the body. Here, the hub coupling part 1164 may be formed in a region inserted into the inner surface of the pitch hub 1170 at one end of the tube body 1161. In addition, the shaft coupling part 1165 may be formed in a region inserted into the inner surface of the straight part 1401 of the connection part 1400 at the other end of the tube body 1161.

The above coupling parts may be in the form of protrusions that partially protrude from the tube body 1161, and a recess that matches the shape of the hub coupling part 1164 may be formed in the pitch hub 1170, and as each hub coupling part 1164 is fitted to the recess, the pitch hub 1170 may be fixedly coupled to the relay tube 1160. Similarly, a recess that matches the shape of the shaft coupling part 1165 is formed in the straight part 1401, and as each shaft coupling part 1165 is fitted to the recess, the straight part 1401 may be fixedly coupled to the relay tube 1160.

In addition, the tube body 1161 of the relay tube 1160 may include one or more protrusions 1163 formed along the circumference of the body. The protrusion 1163 may serve to prevent the cover 1190 from slipping on the relay tube 1160. In detail, the protrusion 1163 may have a band shape, such as a ring, and the protrusion 1163 may be located in approximately the middle region of the tube body 1161 and may also be located on the boundary surface between the pitch hub 1170 and the tube body 1161 and on the boundary surface between the straight part 1401 and the tube body 1161, but the concept of the present disclosure is not limited thereto, and various shapes and numbers of protrusions 1163 may be provided.

In addition, the tube body 1161 of the relay tube 1160 may include a cover coupling part 1162 formed in a region of the body. The cover coupling part 1162 may be coupled to a recess 1192 formed in the cover 1190, which is to be described below, to fixedly couple the cover 1190 to the relay tube 1160 and prevent the cover 1190 from being easily separated from the relay tube 1160, and may serve to guide a position when inserting the cover 1190 into the relay tube 1160.

In this manner, in order to prevent separation of the cover 1190 and guide the position, a plurality of cover coupling parts 1162 may be formed, and the cover coupling parts 1162 may be formed in the shape of a protrusion protruding to have a certain thickness from the tube body 1161. In FIG. 30, the cover coupling parts 1162 are formed on upper and lower surfaces of the relay tube 1160, but the concept of the present disclosure is not limited thereto, and the shape and number of cover coupling parts 1162 may vary.

Hereinafter, the jaw of the end tool 1100 of the sixth embodiment of the present disclosure is described in detail. Here, a component encompassing the first jaw 1101 and the second jaw 1102 may be referred to as the jaw 1103.

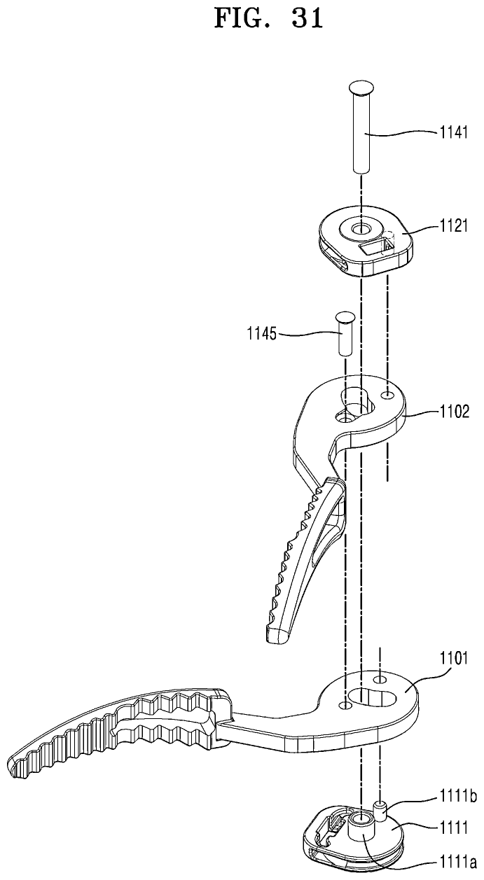

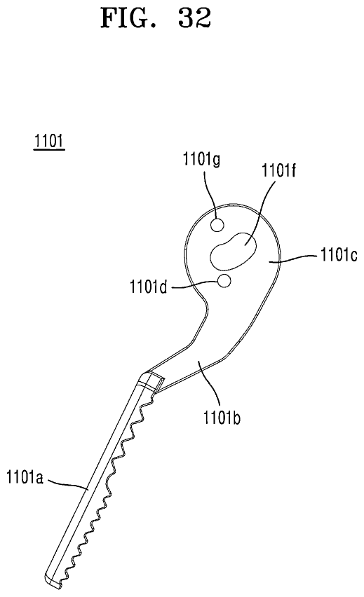

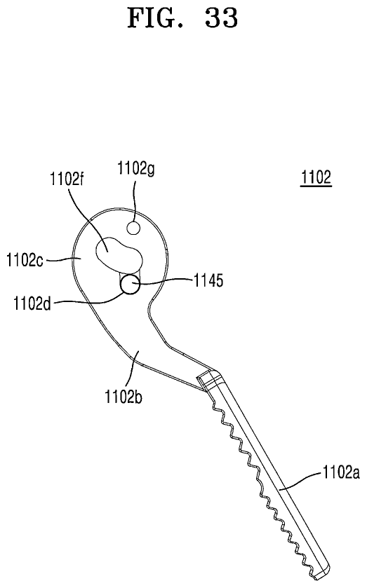

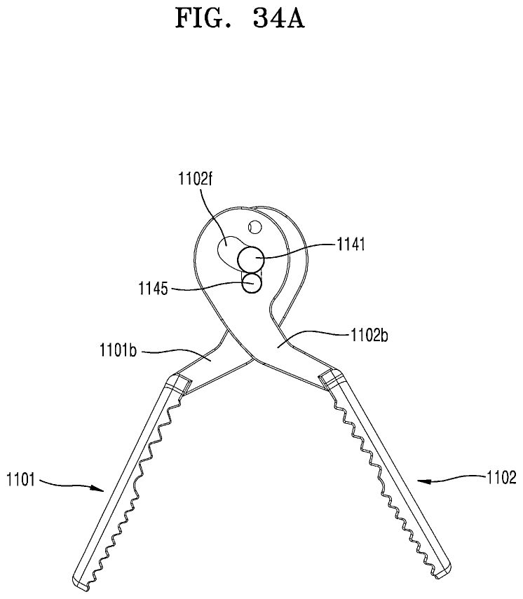

FIG. 31 is an exploded perspective view illustrating a jaw coupling structure of the surgical instrument for electrocautery of FIG. 26, and FIG. 32 is a plan view illustrating the first jaw of FIG. 31. FIG. 33 is a plan view illustrating the second jaw of FIG. 31, and FIGS. 34A and 32B are plan views illustrating coupling of the first and second jaws of FIG. 31.

Referring to FIGS. 31 to 33, the jaw 1103 of the end tool 1100 may be located and coupled between jaw pulleys. That is, the first jaw pulley 1111, the first jaw 1101, the second jaw 1102, and the second jaw pulley 1121 are sequentially stacked and coupled and may be coupled to each other by the rotation shaft 1141 passing through them.

Here, the first jaw 1101 may include a tong part 1101a, an extension part 1101b, and a coupling part 1101c, and second jaw 1102 may include a tong part 1102a, an extension part 1102b, and a coupling part 1102c.

The tong parts 1101a and 1102a are parts that form the body of the jaw 1103, and may have certain irregularities formed on one surface (an inner surface) thereof. In the drawing, a dissector is shown as the tong part, but in addition, various types of tong parts, such as forceps and needle holders, may be provided.

The extension parts 1101b and 1102b are parts that extend from the tong parts 1101a and 1102a in one direction, for example, the −X-axis direction, which is the direction in which the connection part 1400 is formed, and may be formed to form a certain angle with the tong parts 1101a and 1102a. Therefore, when the first jaw 1101 and the second jaw 1102 are coupled through a shaft passing through the coupling parts 1101c 1102c, the extension part 1101b of the first jaw 1101b and the extension part 1102b of the second jaw may be coupled to intersect with each other. In other words, when the first jaw 1101 and the second jaw 1102 are closed, an intersection point 1103b at which the extension part 1101b of the first jaw 1101 and the extension part 1102b of the second jaw 1102 intersect in the shape of X may be formed. From another perspective, when the first jaw 1101 and the second jaw 1102 are closed, a portion of the extension part 1102b adjacent to the coupling part 1102c of the second jaw and the tong part 1101a of the first jaw may be located on one side and a portion of the extension part 1101b adjacent to the coupling part 1101c of the first jaw and the tong part 1102a of the second jaw may be located on the other side, based on the surface (the XZ plane) on which the tong parts 1101a and 1102a are in contact with each other. From another perspective, as shown in FIG. 34B, based on the plane (the XZ plane) on which the tong parts 1101a and 1102a are in contact with each other, the tong part 1101a of the first jaw may be located on one side and at least a portion of the extension part 1101b adjacent to the coupling part 1101c of the first jaw may be located on the other side, and similarly, the tong part 1102a of the second jaw may be located on one side and at least a portion of the extension part 1102b adjacent to the coupling part 1102c of the second jaw may be located on the other side.

Meanwhile, the coupling parts 1101c and 1102c are formed in a flat plane shape at one end of the extension parts 1101b and 1102b and are coupled to the jaw pulleys 1111 and 1121. In addition, the coupling parts 1101c and 1102c may include shaft coupling holes 1101d and 1102d, movement coupling holes 1101f and 1102f, and jaw pulley coupling holes 1101g and 1102g, which are to be described below.

Hereinafter, each component is described based on the first jaw 1101.

Here, the movement coupling hole 1101f is formed to have a certain curvature and may be formed in a substantially oval shape. The shaft coupling part 1111a of the pulley 1111, which is to be described below, may be fitted into the movement coupling hole 1101f. Here, the minor radius of the movement coupling hole 1101f may be substantially equal to or slightly larger than the radius of the shaft coupling part 1111a. Meanwhile, the major radius of the movement coupling hole 1101f may be formed to be larger than the radius of the shaft coupling part 1111a. Therefore, in a state in which the shaft coupling part 1111a of the pulley 1111 is fitted into the movement coupling hole 1101f of the first jaw 1101, the shaft coupling part 1111a may move to a certain extent within the movement coupling hole 1101f.

Meanwhile, the jaw pulley coupling hole 1101g is formed in the shape of a cylindrical hole, and the jaw coupling part 1111b of the pulley 1111, which is to be described below, may be fitted into the jaw pulley coupling hole 1101g. Here, the radius of the jaw pulley coupling hole 1101g may be substantially the same as or slightly larger than the radius of the jaw coupling part 1111b. Accordingly, the jaw coupling part 1111b of the pulley 1111 may be formed to be rotatably coupled to the jaw pulley coupling hole 1101g of the first jaw 1101.

The shaft coupling hole 1101d may be formed at a relatively distal end of the first jaw 1101, that is, on the extension part 1101b side, compared to the movement coupling hole 1101f and the jaw pulley coupling hole 1101g. The shaft coupling hole 1101d is formed in the shape of a hole, and the rotation shaft 1145, which is the jaw rotation shaft, may be inserted through the shaft coupling hole 1101d.

The pulley 1111, which is the first jaw pulley, may include a shaft coupling part 1111a and a jaw coupling part 1111b. The pulley 1111 is formed in an overall rotatable disk shape, and a shaft coupling part 1111a and a jaw coupling part 1111b may be formed to protrude to a certain degree from one surface of the pulley 1111. As described above, the shaft coupling part 1111a of the pulley 1111 may be fitted into the movement coupling hole 1101f of the first jaw 1101, and the jaw coupling part 1111b of the pulley 1111 may be fitted into the jaw pulley coupling hole 1101g of the first jaw 1101. The pulley 1111 may be formed to be rotatable about the rotation shaft 1141, which is an end tool jaw pulley rotation shaft.

Similarly, the shaft coupling hole 1102d, the movement coupling hole 1102f, and the jaw pulley coupling hole 1102g of the coupling part 1102c of second jaw 1102 correspond to the shaft coupling hole 1101d, the movement coupling hole 1101f, and the jaw pulley coupling hole 1101g of the coupling part 1101c of the first jaw, respectively, which are substantially the same with each other, and thus, a detailed description thereof is omitted.

The pulley 1121, which is the second jaw pulley, also includes a shaft coupling part (not shown) and a jaw coupling part (not shown) and is substantially the same as the first jaw pulley 1111 within a corresponding extent, and thus, a detailed description thereof is omitted.

Hereinafter, the amplification of grip force based on a coupling relationship between each of the components described above is described.

In the surgical instrument 60 according to an embodiment of the present disclosure, when the coupling structure of the first jaw 1101 and the second jaw 1102 forms an X-shaped structure and the first jaw 1101 and the second jaw 1102 rotate in a direction toward each other (i.e., when the first jaw 1101 and the second jaw 1102 close), first jaw 1101 and second jaw 1102 are close, the grip force in the direction in which the first jaw 1101 and the second jaw 1102 are close increases further. This is described below in more detail.

As described above, in the operation of opening and closing the first jaw 1101 and the second jaw 1102, there are two axes that become the centers of rotation. That is, the first jaw 1101 and the second jaw 1102 perform opening and closing operations about the two shafts of the rotation shaft 1141 and the rotation shaft 1145. At this time, a rotation center of the first jaw 1101 and the second jaw 1102 is the rotation shaft 1145, and a rotation center of the pulley 1111 and the pulley 1121 is the rotation shaft 1141. At this time, the rotation shaft 1141 becomes a shaft whose position is relatively fixed, and the rotation shaft 1145 becomes a shaft whose position moves in a straight line. In other words, with the position of the rotation shaft 1141 fixed, when the pulley 1111 and pulley 1121 rotate, the rotation shaft 1145, which is the rotation shaft of the first jaw 101 and the second first jaw 102, moves back and forth and the first jaw 1101 and the second jaw 1102 are open/closed.

That is, assuming that the position of the rotation shaft 1141, which is the end tool jaw pulley rotation shaft, is fixed, when the first jaw 1101 and the second jaw 1102 are closed, the rotation shaft 1145, which is the jaw rotation shaft, is forced in an advancing direction (i.e., the distal end direction), and thus, the grip force in the direction in which the first jaw 1101 and the second jaw 1102 are closed increases.

Conversely, when the first jaw 1101 and the second jaw 1102 rotate in the open direction, the rotation shaft 1145 moves backward in the proximal end direction.

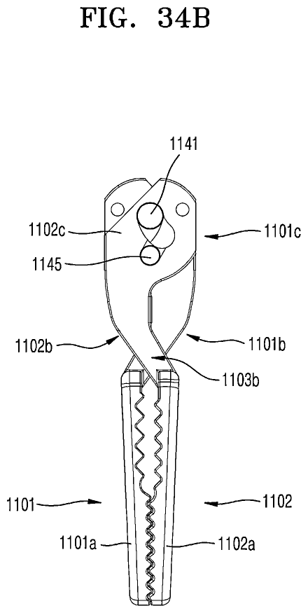

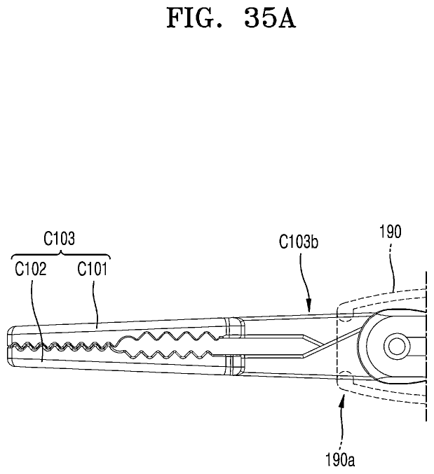

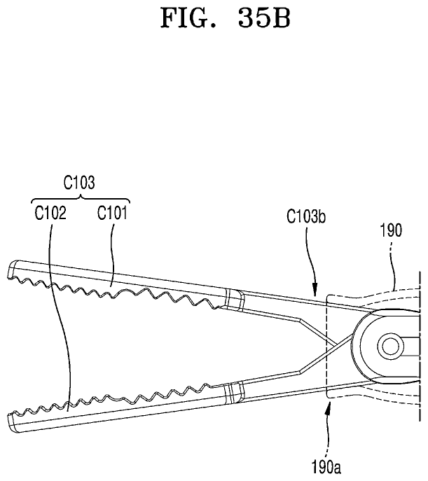

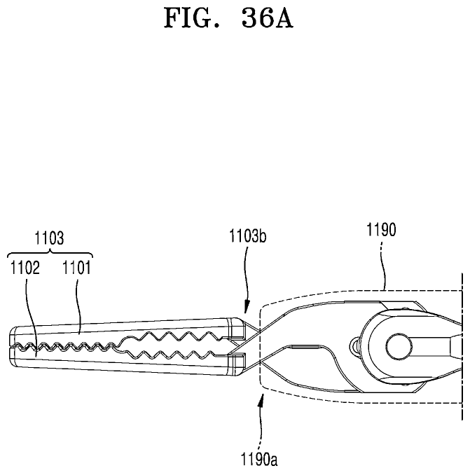

FIGS. 35A and 35B are diagrams illustrating a jaw having an existing structure of a surgical instrument for electrocautery, and FIGS. 36A and 36B are diagrams illustrating a jaw of a surgical instrument for electrocautery of FIG. 23.

Referring to FIGS. 35A and 35B, when the first jaw C101 and the second jaw C102 of the jaw C103 of the existing end tool rotate in the open direction, that is, when each jaw operates to be open, each jaw comes into direct contact with an inlet 190a of the cover 190.

Accordingly, a neck part of the jaw C103 repeatedly contacts the cover 190 and stretches the cover 190, causing the cover 190 to be torn or deformed or the cover 190 to be pushed backward and the insulation is broken. Also, due to this, the angle at which the jaw C103 is open was limited, thereby limiting the opening and closing operation of the jaw.

In contrast, referring back to FIG. 34B, the jaw 1103 of the end tool 1100 in the sixth embodiment of the present disclosure is formed in an X-shaped structure and includes the jaw rotation shaft 1145 inserted through the first jaw 1101 and the second jaw 1102 to become the rotation center of the first jaw 1101 and the second jaw 1102 and the intersection point 1103b at which the extension part 1101b of the first jaw 1101 and the extension part 1102b of the second jaw 1102 intersect in an X shape when the first jaw 1101 and the second jaw 1102 are closed, and the intersection point 1103b is formed apart from the jaw rotation shaft 1145.

In addition, referring to FIGS. 36A and 36B, an inlet part 1190a of the cover 1190 may be located adjacent to the intersection point 1103b at which the extension part 1101b of the first jaw and the extension part 1102b of the second jaw intersect. Accordingly, even when the first jaw 1101 and the second jaw 1102 rotate in the open direction, the first jaw 1101 and the second jaw 1102 may not directly contact the cover 1190. That is, even if the jaw 1103 is open, no force is applied to expand the inlet of the cover 1190, and damage, such as deformation or tearing of the cover 1190, due to repeated opening and closing operations of the jaw 1103 may be prevented. In addition, even if the jaw 1103 is open, the cover 1190 is not pushed backward, so the insulation may be stably maintained.

In other words, because the jaw 1103 is formed to have the X-shaped structure and the inlet portion 1190a of the cover 1190 is located at the intersection point 1103b of the X-shaped neck portion of the jaw 1103, a limitation to the opening and closing angle of the jaw 1103 may be reduced and contact between the jaw 1103 and the cover 1190 may be prevented, thereby preventing deformation of the cover 1190 or destruction of insulation.

Hereinafter, the cover 1190 of the end tool 1100 according to the sixth embodiment of the present disclosure is described in detail.

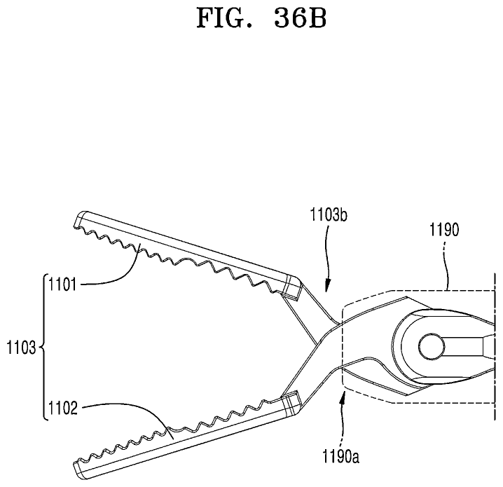

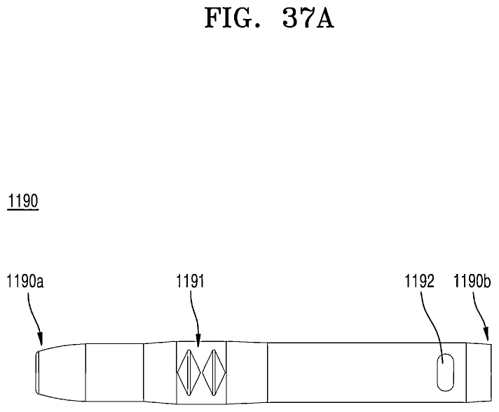

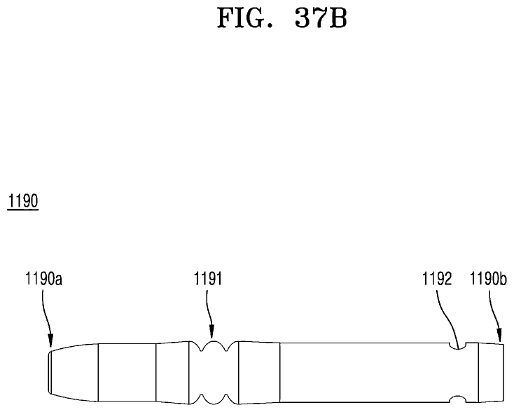

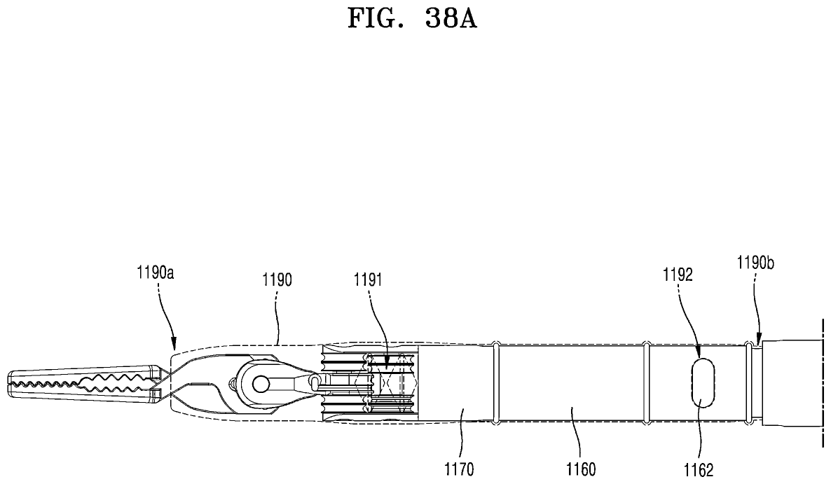

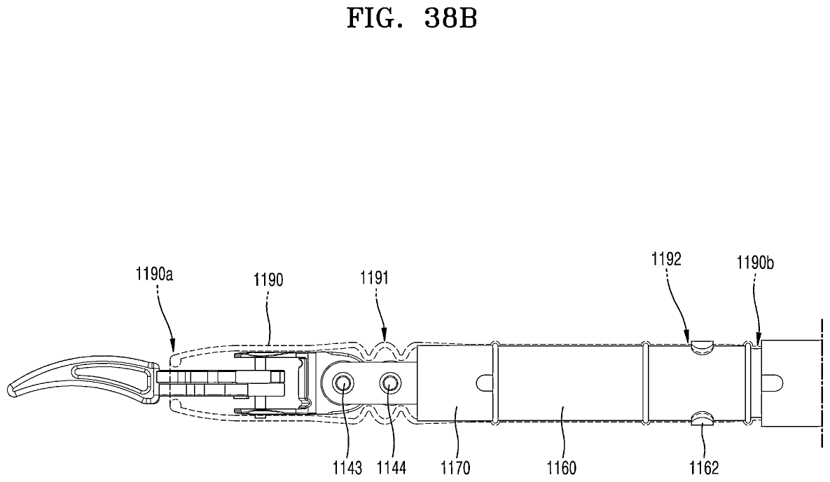

FIG. 37A is a plan view illustrating the cover of the surgical instrument for electrocautery of FIG. 23, and FIG. 37B is a side view illustrating the cover. FIG. 38A is a plan view illustrating the end tool and the cover on the end tool of the surgical instrument for electrocautery of FIG. 23, and FIG. 38B is a side view of the end tool.

The cover 1190 is a cover that surrounds a significant portion of the end tool 1100, including the relay tube 1160 described above. Specifically, the cover 1190 may be considered to be a cover that covers a certain region from the relay tube 1160 to the pitch hub 1170, the end tool hub 1180, and the jaw 1103. The cover 1190 may be formed of an insulating material. For example, the cover 1190 may be formed of silicone rubber, neoprene rubber, polytetrafluoroethylene (PTFE), polyurethane, epoxy, etc.

Referring to FIGS. 37A and 37B, the cover 1190 includes wrinkles 1191 and recess 1192 in a body. The wrinkles 1191 may be formed on the cover 1190 in a certain region in which a pitch motion of the end tool 1100 occurs. That is, the wrinkles 1191 may be partially formed in a direction parallel to the rotation shaft on a surface opposite to the rotation shaft of the pitch hub 1170 at which the pitch motion occurs. In detail, the pitch hub 1170 includes a third pin 1143 that is axially coupled to the end tool hub 1180 and functions as an end tool pitch rotation shaft and a fourth pin 1144 that functions as an end tool pitch auxiliary rotation shaft. The fourth pin 1144 is formed parallel to the third pin 1143 and is formed on one side of the third pin 1143. At this time, the wrinkles 1191 of the cover 1190 may be formed in a region relatively closer to the fourth pin 1144 than the third pin 1143. In other words, the wrinkles 1191 of the cover 1190 are considered to be formed closer to the fourth pin 1144 than the third pin 1143. Accordingly, when the cover 1190 is pulled during the pitch motion of the end tool 1100 and the wrinkles 1191 are straightened, the wrinkles 1191 may be located on the third pin 1143, which is the end tool pitch rotation shaft.

Meanwhile, the recess 1192 of the cover 1190 is formed in a location corresponding to the cover coupling part 1162 of the aforementioned relay tube 1160 and may be coupled to the cover coupling part 1162. In other words, the cover coupling part 1162 protruding from the relay tube 1160 may be inserted into the recess 1192 of the cover 1190.

In addition, the cover 1190 may be formed to be inclined as the diameter of an end 1190b adjacent to the proximal end decreases toward the proximal end.

Accordingly, a step may be prevented from occurring at the boundary between the cover 1190 and the straight part 1401.

In addition, as shown in FIG. 37A or the like, the diameter of the body of the cover 1190 may be slightly smaller than the diameter of the relay tube 1160.

Accordingly, the cover 1190 may be coupled to the relay tube 1160 by an interference fit to prevent the cover 1190 from escaping from the end tool 1100.

Hereinafter, an operation according to intuitive driving of the surgical instrument for electrocautery of FIG. 23 according to the sixth embodiment of the present disclosure is described.

The intuitive driving of the present disclosure has been described in detail through the first embodiment, and because the present embodiment is also substantially the same, detailed description thereof is omitted.

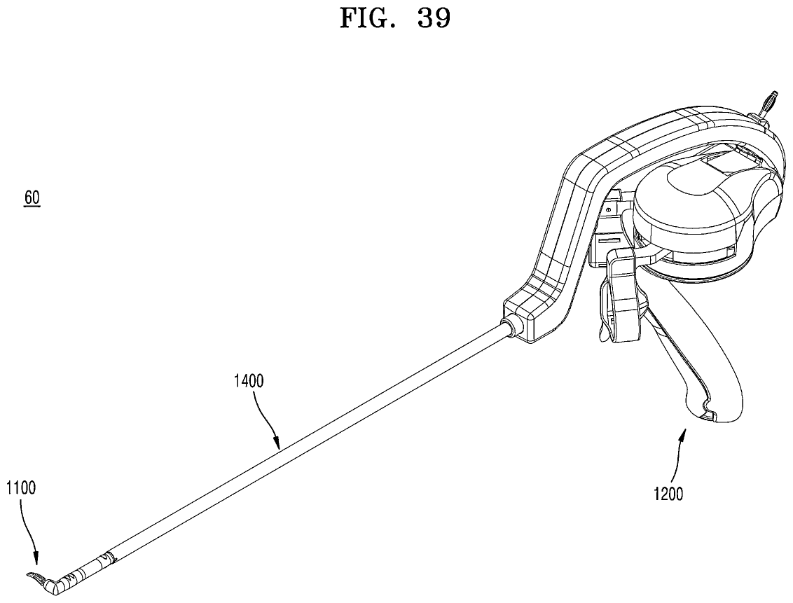

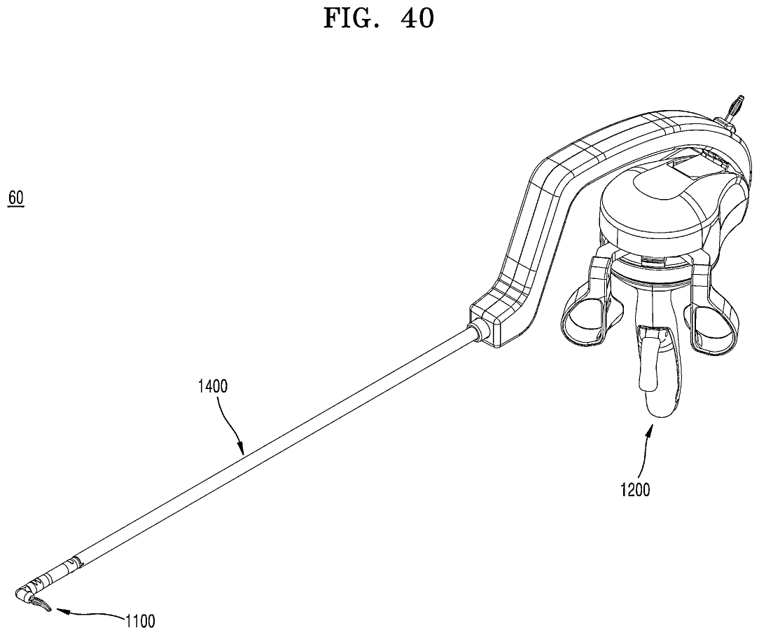

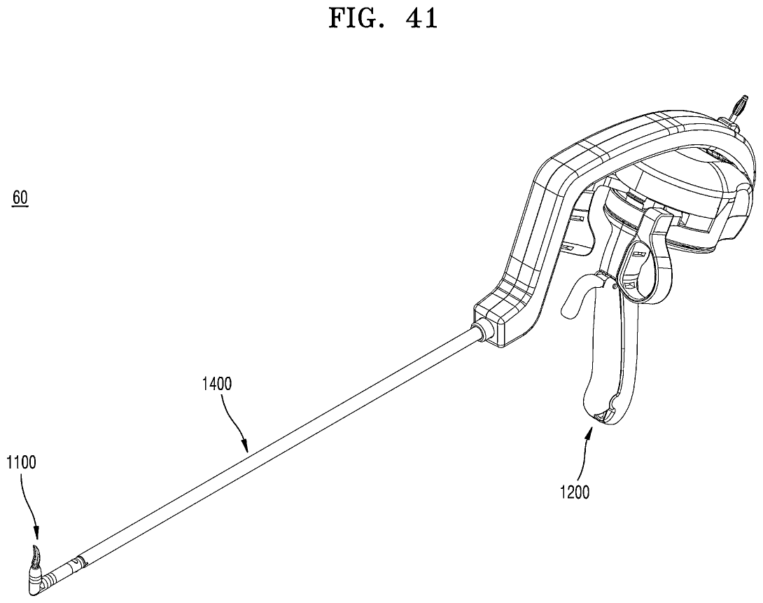

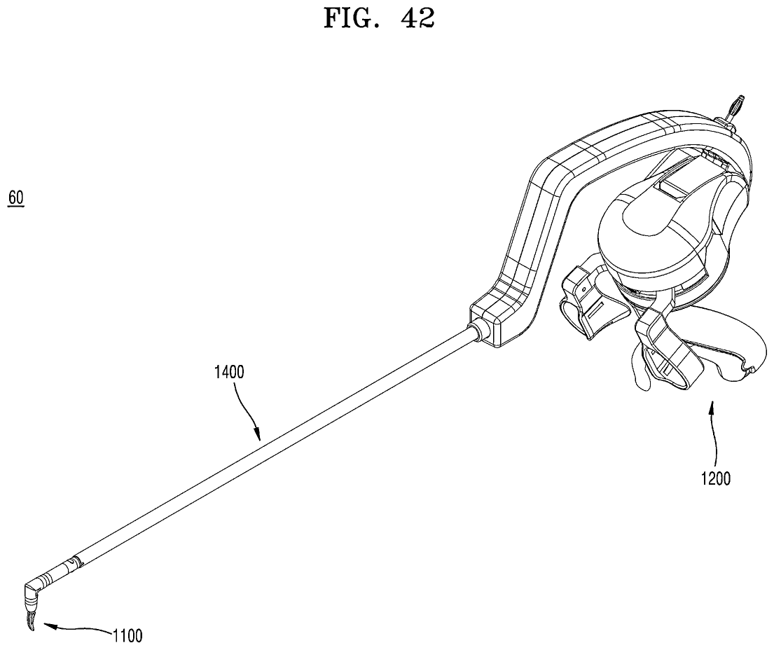

FIGS. 39 and 40 are perspective views illustrating a yaw motion of the surgical instrument for electrocautery of FIG. 23, FIGS. 41 and 42 are perspective views illustrating a pitch motion of the surgical instrument for electrocautery of FIG. 23, and FIG. 43 is a perspective view illustrating a yaw-pitch motion of the surgical instrument for electrocautery of FIG. 23.

Here, FIG. 39 is a diagram illustrating a state in which the jaws are yaw-rotated by +90°, and FIG. 40 is a diagram illustrating a state in which the jaws are yaw-rotated by −90°.

Referring to FIGS. 39 and 40, it can be seen that, when performing the yaw motion, the operations of the manipulation part 1200 and the end tool 1100 intuitively coincide. That is, when the manipulation part 1200 rotates in the + direction based on a yaw rotation shaft (a Z-axis), the end tool 1100 also rotates in the + direction based on the yaw rotation shaft (the Z-axis). In addition, when the manipulation part 1200 rotates in the − direction based on the yaw rotation shaft (the Z-axis), the end tool 1100 also rotates in the − direction based on the yaw rotation shaft (the Z-axis). Here, a rotation angle of the manipulation part 1200 and a rotation angle of the end tool 1100 may be set to vary depending on the ratio of the pulleys.

FIG. 41 is a diagram illustrating a state in which the jaws are pitch-rotated by +90°, and FIG. 42 is a diagram illustrating a state in which the jaws are pitch-rotated by −90°.

Referring to FIGS. 41 and 42, it can be seen that, when performing a pitch motion, the operations of the manipulation part 1200 and the end tool 1100 intuitively coincide. That is, when the manipulation part 1200 rotates in the + direction based on a pitch rotation shaft (a Y-axis), the end tool 1100 also rotates in the + direction based on the pitch rotation shaft (the Y-axis). In addition, when the manipulation part 1200 rotates in the − direction based on the pitch rotation shaft (the Y-axis), the end tool 1100 also rotates in the − direction based on the pitch rotation shaft (the Y-axis). Here, a rotation angle of the manipulation part 1200 and a rotation angle of the end tool 1100 may be set to vary depending on the ratio of the pulleys.

FIG. 43 is a diagram illustrating a state in which the jaws are pitch-rotated by +90° and yaw-rotated by −90°.

Referring to FIG. 43, it can be seen that, even when performing the pitch motion and the yaw motion simultaneously, the operations of the manipulation part 1200 and the end tool 1100 intuitively coincide.

Here, the intuitively same direction may be further explained as meaning that a movement direction of the user's fingers holding the manipulation part 1200 and a movement direction of an end of the end tool 1100 are substantially the same. Of course, the same direction here may not refer to a direction that perfectly matches on the three-dimensional coordinates, and may be understood as the sameness to the extent that, for example, when the user's finger moves to the left, the distal end of the end tool 1100 also moves to the left, and when the user's finger moves to the left, the distal end of the end tool 1100 moves downward, the distal end of the end tool 1100 also moves downward.

### <Seventh Embodiment> (Monopolar Scissor)

Hereinafter, an end tool 2100 of a surgical instrument 70 for electrocautery according to the seventh embodiment of the present disclosure is described. Here, the end tool 2100 of the surgical instrument for electrocautery according to the seventh embodiment of the present disclosure is different in the configuration of the jaw from the end tool (see 1100 of FIG. 23 or the like) of the surgical instrument according to the sixth embodiment of the present disclosure described above. The configuration different from the sixth embodiment is described in detail below.

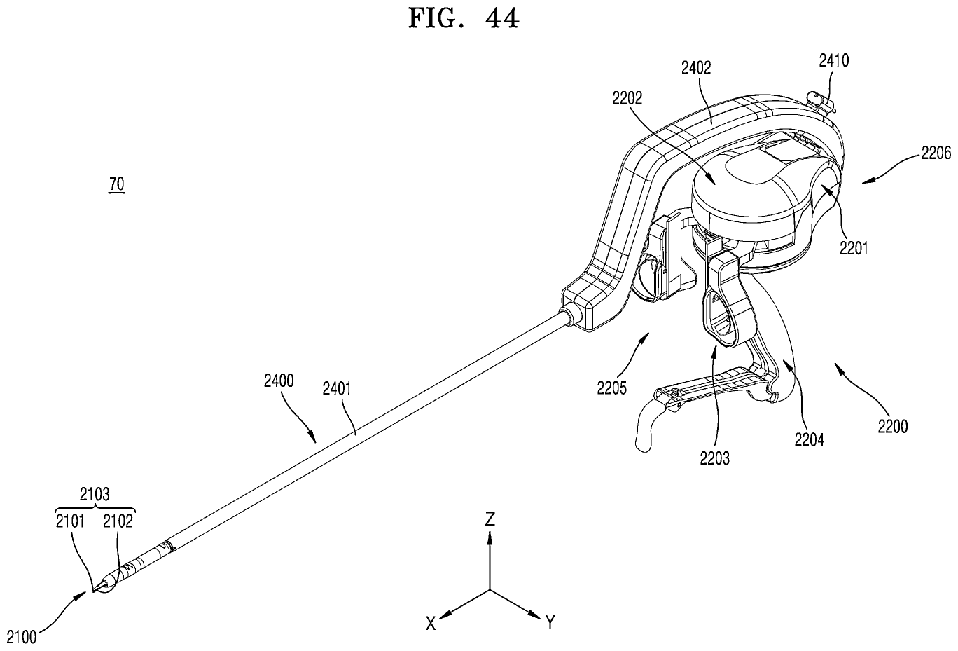

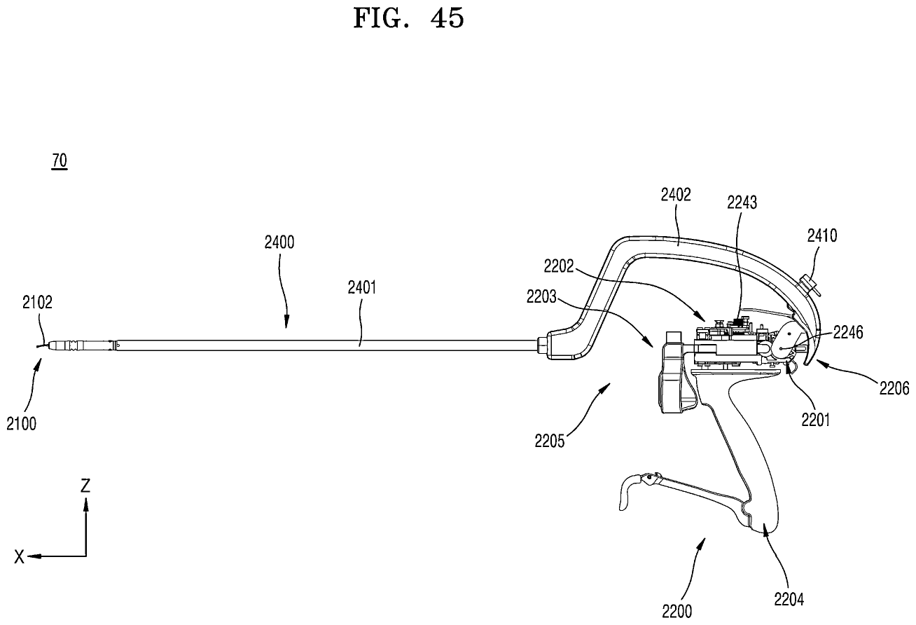

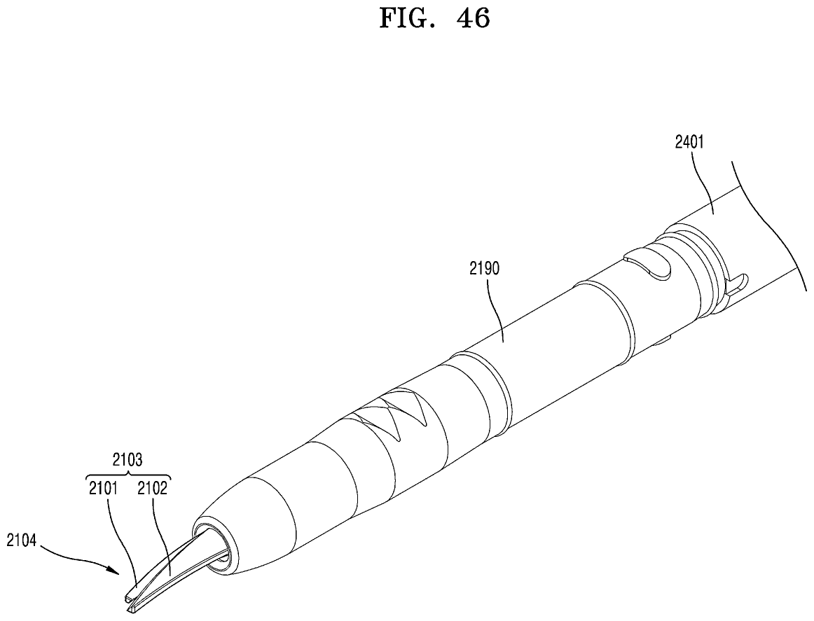

FIG. 44 is a perspective view illustrating a surgical instrument for electrocautery according to a seventh embodiment of the present disclosure, and FIG. 45 is a side view illustrating the surgical instrument for electrocautery of FIG. 44. FIGS. 46 and 47 are perspective views illustrating an end tool of the surgical instrument for electrocautery of FIG. 44.

First, referring to FIGS. 44 and 45, the surgical instrument 70 for electrocautery according to the seventh embodiment of the present disclosure includes an end tool 2100, a manipulation part 2200, a power transmission part (not shown), and a connection part 2400.

The connection part 2400 has a straight part 2401 and a bent part 2402, and includes a connector 2410. The manipulation part 2200 includes a first handle 2204, an actuation manipulation part 2203, a yaw manipulation part 2202, and a pitch manipulation part 2201.

In the present embodiment, each configuration of the connection part 2400 and the manipulation part 2200 are substantially the same as the connection part 2400 and the manipulation part 2200 described above with reference to FIG. 2 or the like in the first embodiment, and therefore, a detailed description thereof is omitted here.

FIG. 46 is a perspective view illustrating the end tool 2100 including a cover 2190, and FIG. 47 is a perspective view illustrating the end tool 2100 of FIG. 46 without the cover 2190.

Referring to FIGS. 46 and 47, the end tool 2100 of the surgical instrument 70 for electrocautery according to the seventh embodiment of the present disclosure includes a pair of jaws, that is, a first jaw 2101 and a second jaw 2102. Here, the first jaw 2101 and second jaw 2102 separately or a component encompassing the first jaw 2101 and the second jaw 2102 may be referred to as a jaw 2103. The first jaw 2101 and the second jaw 2102 are described in detail below.

In addition, the end tool 2100 may include an end tool hub 2180, a pitch hub 2170, and a relay tube 2160. These components may be sequentially coupled in the direction from the distal end 2104 to the proximal end 2105 of the end tool, and the relay tube 2160 may be connected to a shaft, which is a straight part 2401 of the connection part 2400.

In the end tool 2100 of the present embodiment, a rotation shaft 2141 may be inserted through the end tool hub 2180, and a rotation shaft 2143 and a rotation shaft 2144 may be inserted through the pitch hub 2170.

In the present embodiment, the end tool hub 2180, the pitch hub 2170, and each of the rotation shafts 2141, 2143, and 2144 are substantially the same as the end tool hub 2180, the pitch hub 2170, and each of the rotation shafts 2141, 2143, and 2144, and thus, a detailed description thereof is omitted here.

In addition, the relay tube 2160 and the cover 2190 of the present embodiment are substantially the same as the relay tube 2160 and the cover 2190 described above with reference to FIG. 30 of the sixth embodiment, and thus, a detailed description thereof is omitted here.

Meanwhile, although not shown in the drawings, the surgical instrument 70 for electrocautery according to the seventh embodiment of the present disclosure may further include the wire (see 301 of FIG. 16), the shrink tube (see 331 of FIG. 16), and the shaft tube (see 341 of FIG. 16), like the surgical instrument for electrocautery 10 according to the first embodiment of the present disclosure shown in FIGS. 2 and 16 or the like. In addition, in the surgical instrument 70 according to the seventh embodiment of the present disclosure, the wire (see 301 of FIG. 16) is inserted into the shrink tube (see 331 of FIG. 16) and the shrink tube (see 331 of FIG. 16) is inserted into the shaft tube (see 341 of FIG. 16), so that the wire (see 301 of FIG. 16) is not in direct contact with the shaft tube (see 341 of FIG. 16) and is insulated therefrom.

Hereinafter, the jaw of the end tool 2100 of the seventh embodiment of the present disclosure is described in detail. Here, the component encompassing the first jaw 2101 and the second jaw 2102 may be referred to as the jaw 2103.

Although not shown in FIG. 47, the jaw according to the present embodiment includes a coupling part as described in the sixth embodiment, and the coupling part is coupled to the jaw pulleys 2111 and 2121. In addition, the coupling part may include a shaft coupling hole, a movement coupling hole, and a jaw pulley coupling hole.

The shaft coupling part of the pulley may be fitted into the movement coupling hole, and the jaw coupling part of the pulley may be fitted into the jaw pulley coupling hole. The rotation shaft 2141, which is the jaw pulley rotation shaft, passes through the movement coupling hole, and the rotation shaft 2145, which is the jaw rotation shaft, passes through the shaft coupling hole.

Here, the jaw pulleys 2111 and 2121, the coupling part, the shaft coupling hole, the movement coupling hole, the jaw pulley coupling hole, and the rotation shaft 2141 and 2145 of the present embodiment are substantially the same as the jaw pulleys 2111 and 2121, the coupling parts 2101c and 2102c, the shaft coupling holes 2101d and 2102d, the movement coupling holes 2101f and 2102f, and the jaw pulley coupling holes 2101g and 2102g, and thus, a detailed description thereof is omitted here.

The end tool 2100 according to the seventh embodiment of the present disclosure is different in shape of the jaw 2103 from the end tool 1100 according to the sixth embodiment of the present disclosure described above.

The jaw 2103 of the end tool 2100 according to the seventh embodiment of the present disclosure is to cut an object, rather than gripping an object, and functions as scissors. Therefore, in a state in which the jaw 2103 is closed, the first jaw 2101 and the second jaw 2102 may overlap each other, and the inner surface of the jaw is formed with a sharp blade.

As such, the present disclosure has been described with reference to an embodiment shown in the drawings, but this is merely an example, and those skilled in the art will understand that various modifications and variations of the embodiment are made therefrom. Therefore, the true scope of technical protection of the present disclosure should be determined by the technical spirit of the enclosed patent claims.

According to the present disclosure, the insulation performance of the surgical instrument for electrocautery may be improved.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A surgical instrument for electrocautery, the surgical instrument comprising: an end tool including one or more jaws and configured to be rotatable in two or more directions;
a connection part extending in a first direction (an X-axis) and having one end to which the end tool is coupled;
a wire having one end connected to the end tool;
a shrink tube into which the other end of the wire, opposite to the one end of the wire connected to the end tool, is inserted; and
a shaft tube, into which the other end of the shrink tube, opposite to one end of the shrink tube into which the wire is inserted, is inserted,
wherein the wire and the shaft tube are separated by the shrink tube, and the wire and the shaft tube are insulated from each other.

2. The surgical instrument of claim 1, wherein
the other end of the wire is not exposed to the outside of the other end of the shrink tube.

3. The surgical instrument of claim 1, wherein
a region in which the wire, the shrink tube, and the shaft tube overlap is crimped.

4. The surgical instrument of claim 1, wherein,
in a state in which the wire is inserted into the shrink tube, the shrink tube is heated and contracted to be tightly coupled to the wire.

5. The surgical instrument of claim 1, further comprising:
a manipulation part configured to control rotation of the end tool in the two or more directions, and
the manipulation part is coupled to the other end of the connection part.

6. The surgical instrument of claim 5, wherein
the shaft tube connects the wire at the side of the end tool to the wire at the side of the manipulation part.

7. The surgical instrument of claim 1, wherein
the shaft tube is disposed within the connection part.

8. The surgical instrument of claim 1, wherein
the wire and the shaft tube are not in direct contact.

9. The surgical instrument of claim 1, wherein
the wire, the shrink tube, and the shaft tube are each provided in plurality, and
the plurality of wires and the plurality of shaft tubes are formed not to directly face each other in any operating state.

10. The surgical instrument of claim 1, further comprising:
an electric wire connected to the jaw to supply electrical energy to the jaw,
wherein a voltage induced in the shaft tube when electric energy flows through the electric wire is blocked from being transmitted to the wire by the shrink tube.

11. The surgical instrument of claim 1, wherein
the entire wire is formed within the shrink tube.

12. The surgical instrument of claim 1, wherein
the end tool includes:
an end tool hub axially coupled so that an end tool jaw pulley to which the jaw is coupled is rotatable; and
a pitch hub axially coupled to the end tool hub by a rotation shaft coupled to one end of the end tool hub,
wherein the end tool hub and the pitch hub include an insulator.

13. The surgical instrument of claim 12, wherein
a whole surface of the end tool hub and the pitch hub includes an insulating material.

14. The surgical instrument of claim 1, wherein
the end tool includes:
an end tool hub axially coupled so that an end tool jaw pulley to which the jaw is coupled is rotatable;
a pitch pulley axially coupled to a pitch rotation shaft formed at one end of the end tool hub;
a pitch wire wound around the pitch pulley; and
a fastening member coupled to the pitch wire to couple the pitch pulley to the pitch wire,
wherein the end tool hub and the fastening member are separated by the pitch pulley and the end tool hub and the fastening member are insulated from each other.

15. The surgical instrument of claim 14, wherein
the pitch wire is formed as a single wire, and the fastening member is fastened to a portion of the pitch wire.

16. The surgical instrument of claim 14, wherein
the pitch pulley includes an insulator.

17. The surgical instrument of claim 14, wherein
the pitch pulley includes at least one coupling part fixedly coupling the pitch pulley to the end tool hub.

18. The surgical instrument of claim 14, wherein
the fastening member is not in direct contact with the end tool hub.
